(19) United States
(12) Patent Application Publication
(10) Pub. No.: US 2024/0026305 A1
(43) Pub. Date: Jan. 25, 2024

(54) RECOMBINANT ONCOLYTIC VIRUSES, SURFACE-ENGINEERED DELIVERY SYSTEMS AND RELATED METHODS

(71) Applicant: DEVACELL, INC., San Diego, CA (US)

(72) Inventor: Inanc ORTAC, San Diego, CA (US)

(21) Appl. No.: 18/254,138

(22) PCT Filed: Nov. 23, 2021
(86) PCT No.: PCT/US2021/060669

§ 371 Date: May 23, 2023

Related U.S. Application Data
(60) Provisional application No. 63/117,431, filed on Nov. 23, 2020.

(51) Int. Cl. C12N 7/00; A61P 35/00

(52) U.S. Cl. CPC C12N 7/00; A61P 35/00; C12N 2710/10022; C12N 2710/10043

(57) ABSTRACT

Provided herein are recombinant viruses and artificially coated delivery systems, and methods of use.

### TECHNICAL FIELD

The invention relates to novel recombinant viruses, artificially coated delivery systems, and related methods.

### BACKGROUND

There is a need for novel systemic delivery systems for pharmaceutical payloads, including recombinant viruses, nucleic acid vaccines, gene therapies and oncolytic virotherapies.

### SEQUENCE LISTING

The sequence listing submitted herewith in the ASCII text file entitled, “128254-0007WO01_ST25-PCT”, created on Nov. 23, 2021, with a file size of 55.8 KB, is incorporated herein by reference in its entirety.

### SUMMARY

Provided herein is a replication-competent oncolytic virus comprising a replication-competent oncolytic virus comprising: a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme. In particular embodiments, each transgene expresses a different protein. In other embodiments, the first transgene expresses GM-CSF, IL-12, IL-15, TNF-α, or 4-1BBL. In yet another embodiment, the second transgene expresses CCL4, CCL5/RANTES, CXCL11, 4-1BBL, GM-CSF, or TNF-α. In another embodiment, the third transgene expresses an essential-agent-depletion-enzyme selected from methioninase, adenosine deaminase, or cytosine deaminase, asparaginase or uricase. In a particular embodiment, the first transgene expresses GM-CSF, IL-12, IL-15, TNF-α, or 4-1BBL; the second transgene expresses CCL4, CCL5/RANTES, CXCL11, 4-1BBL, GM-CSF, or TNF-α; and the third transgene expresses methioninase, adenosine deaminase, or cytosine deaminase. In a particular embodiment, the third transgene is expressed before the other two transgenes. In further embodiments, the third transgene is expressed under control of an E1 or E3 promoter.

In certain embodiments, the virus is selected from the group consisting of: adeno-associated virus (AAV), adenovirus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus, Enadenotucirev oncolytic virus, Ad26, Imlygic, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus, Pexa-Vec, Reolysin, DS-1647, TG1042, Cavatak, GL-ONC1, Marabex, ORCA-010, ParvOryx, LOAd703, PV701, MV-NIS, ONCOS-102, Seprehvir, Enadenotucirev, CG0070, Telomelysin, JX-929, VSV Cancer Project, Ad-VirRx 007, NG-348, VSV-GP, RP1/RP2/RP3, WO-12, Maraba virus, Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, Bovine ephemeral fever virus and/or Dimarhabdovirus.

In a particular embodiment, the virus is an adenovirus. In yet another embodiment, the adenovirus further comprises a 24-bp deletion in E1A Conserved Region 2 (CR2), and a 2428 bp BsiWI-BglII deletion in the E3 region. In particular embodiments, the first, second, and third transgenes are selected from the group of first, second and third transgene combinations set forth in Table 1 and FIGS. 19-21. In another embodiment, the first, second, and third transgenes are selected from the group of first, second and third transgene combinations selected from:

| | | | |
|---|---|---|---|
| Ad5.DEV09 | RFP | hCCL5/RANTES-human | IL15-human |
| Ad5.DEV10 | Adenosine Deaminase-human | hCCL5/RANTES-human | IL15-human |
| Ad5.DEV11 | Methioninase | hCCL5/RANTES-human | IL15-human |
| Ad5.DEV12 | Methioninase | Adenosine Deaminase-human | IL15-human |
| Ad5.DEV13 | Methioninase | Adenosine Deaminase-human | GM-CSF-mouse |
| Ad5.DEV14 | Adenosine Deaminase-human | GM-CSF-mouse | IL15-human |
| Ad5.DEV15 | Methioninase | GM-CSF-mouse | IL15-human |

In another embodiment, provided herein is a replication-competent oncolytic virus comprising a first, and a second transgene, whereas each transgene expresses GM-CSF, TNF-α, 4-1BBL, CCL4, CCL5/RANTES, CXCL11, or an essential-agent-depletion-enzyme, wherein each transgene expresses a different protein, and wherein the first and second transgenes are selected from the group of first and second transgene combinations set forth in Table 1 and FIGS. 19-21.

Provided herein is a surface-engineered delivery system, said system comprising: a payload; and an artificial coating layer surrounding the payload, wherein the artificial coating is selected from the group consisting of: silica, titanium oxide and calcium phosphate.

In an embodiment, the payload is a recombinant virus having a recombinant genome or the recombinant genome itself, and the artificial coating layer surrounds the recombinant virus and/or recombinant genome.

In particular embodiments, the surface-engineered recombinant virus is at least one selected from the group consisting of: oncolytic viruses, enadenotucirev oncolytic virus, Poxviruses (including but not limited to VV), vaccinia virus, Herpes Virus, herpes simplex virus-1, herpes simplex virus-2, Rhabdoviruses (including but not limited to Rabies, VSV), Vesicular stomatitis virus, Coronavirus, SARS-CoV-2, Hepadnaviruses, Asfarviridae, Flaviviruses (including but not limited to Kunjin, West Nile, Dengue), Alphaviruses (including but not limited to SFV, SIN, VEE, MD, Togavirus, Hepatitis D virus, Orthomyxoviruses, Paramyxoviruses, Bunyaviruses, Filoviruses, Retroviruses (including but not limited to MMSV, MSCV), Noroviruses, adenoviruses (including but not limited to Ad5), adeno-associated viruses (including but not limited to AAV1, 2, 3, 4, 5, 6, 7, 8, 9), lentiviruses (including but not limited to HIV-1, HIV-2), measles virus, Newcastle disease virus, rotaviruses, poliovirus, Picornaviruses, enteroviruses, rhinoviruses, Coxsackie viruses, echoviruses, and hepatitis A virus. In other embodiments, the surface-engineered recombinant virus is an oncolytic virus and is selected from the group consisting of: NG-641 (PsiOxus), and Imlygic (talimogene laherparepvec). In a yet further embodiments, the recombinant virus and/or recombinant viral genome is used in a vaccine selected from AZD1222 (AstraZeneca), ChAdOx1-nCov19 (Oxford), Ad5-nCoV (CanSino), VSV, and Ad26 (J&J).

In another embodiment, the surface-engineered recombinant virus is a vaccine and the virus is a replication deficient Ad5 (Human) Adenovirus vector, wherein the recombinant genome encodes SARS-CoV-2 spike protein and the E1 & E3 genes are deleted. In yet another embodiment, the surface-engineered recombinant virus is oncolytic and the virus is VSV.

In another embodiment, the payload is at least one nucleic acid selected from mRNA, siRNA, miRNA, small nuclear RNA, plasmid DNA, antisense oligodeoxynucleotides, or any other nucleic acid known to those skilled in the art. In a further embodiment, the nucleic acid is complexed with a cation to form a polyion complex. In yet further embodiments the nucleic acid is selected from that contained in patisiran (Alnylam), BNT162b2 (Pfizer-BioNTech), and mRNA-1273 (Moderna).

In certain embodiments, the artificial coating is applied by conducting a charge-mediated sol-gel condensation reaction directly onto the surface of the payload. In further embodiments, the artificial coating comprises a silica gel matrix or titanium oxide gel matrix encapsulating the payload. In particular embodiments of the invention surface-engineered delivery system, the artificial coating comprises a cell-surface ligand selected from the group consisting of: proteins, polysaccharides, aptamers, peptides, oligonucleotides and small molecules. In other embodiments, the artificial coating comprises at least one targeting ligand selected from the group consisting of: Antibodies, transferrin, Hyaluronic acid, RGD (e.g., cRGD), IL4RPep-1, AS-1411, GBI-10, Folate, anisamide, and phenylboronic acid. In other embodiments, of the invention surface-engineered delivery system, the recombinant virus can be replication-competent or replication-defective. In certain embodiments, the recombinant virus has had its native envelope removed prior to coating with the artificial coating layer. In certain embodiments, the recombinant virus is natively non-enveloped.

Also provided herein is a surface-engineered recombinant virus vaccine, said virus comprising:

a recombinant Ad5 virus having a recombinant genome encoding SARS-CoV-2 spike protein, wherein at least a functional portion of E1 and E3 genes are deleted or wherein the genome does not contain any native Ad5 viral genes (i.e., “gutted” Ad5), and an artificial coating layer encapsulating the recombinant Ad5 virus, wherein said coating layer comprises an effective amount of a ligand to bind to a cell, and wherein the artificial coating is selected from the group consisting of: silica, titanium oxide and calcium phosphate.

Also provided herein is a method of making a surface-engineered recombinant virus, said method comprising:
producing a recombinant virus having a recombinant genome; and
applying an artificial coating to the recombinant virus, wherein the artificial coating is selected from the group consisting of: silica, titanium oxide and calcium phosphate. In particular embodiments, the artificial coating is applied by conducting a charge-mediated sol-gel condensation reaction directly onto the surface of the recombinant virus. In certain embodiments, the artificial coating comprises a silica gel matrix or titanium oxide gel matrix encapsulating the recombinant virus. In another embodiment, the recombinant virus has had it native-envelope removed prior to coating with the artificial coating layer. In another embodiment, the recombinant virus is natively non-enveloped.

Also provided herein, is a method of re-engineering the surface of a virus having a native-envelope, said method comprising:
removing the native-envelope from the virus to isolate a previously-enveloped-capsid; and applying an artificial coating to the previously-enveloped-capsid. Any enveloped virus known in the art is suitable for use herein. In certain embodiments, the enveloped virus can be selected from the group consisting of: Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus. In particular embodiments, the native-envelope is removed or delipidated using a detergent and/or an extraction solvent. In some embodiments, the detergent and/or an extraction solvent can be selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination. In certain embodiments, the artificial coating is selected from the group consisting of: silica, titanium oxide and calcium phosphate. In one embodiment, applying the artificial coating further comprises conducting a charge-mediated sol-gel condensation reaction directly onto the surface of the previously-enveloped-capsid. In another embodiment, the artificial coating comprises a silica gel matrix or titanium oxide gel matrix encapsulating the previously-enveloped-capsid. In particular embodiments, the previously-enveloped-capsid is replication-competent or replication-defective.

Also provided herein is a surface-re-engineered virus comprising:

a previously-enveloped-capsid from a naturally occurring enveloped-virus; and an artificial coating layer surrounding the previously-enveloped-capsid. Any enveloped virus known in the art is suitable for use herein. In particular embodiments, the envelope virus can be selected from the group consisting of: Poxvirus, vaccinia virus, Herpes Virus, herpes simplex virus-1, herpes simplex virus-2, Rhabdovirus, Vesicular stomatitis virus (VSV), Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus. As set forth herein, the artificial coating is selected from the group consisting of: silica, titanium oxide and calcium phosphate. In certain embodiments, the artificial coating is applied by conducting a charge-mediated sol-gel condensation reaction directly onto the surface of the previously-enveloped-capsid. In other embodiments, the artificial coating comprises a silica gel matrix or titanium oxide gel matrix encapsulating the previously-enveloped-capsid. The previously-enveloped-capsid can be either replication-competent or replication-defective.

Also provided herein is a method of re-engineering a virus having a native-envelope, said method comprising: removing the native-envelope surrounding a capsid from the virus; isolating the previously-enveloped-capsid; and applying an artificial coating to the previously-enveloped-capsid.

Also provided herein is a composition comprising:

a capsid from a native envelope-virus, wherein the capsid is devoid of its native envelope; and an artificial coating-layer, wherein the coating-layer encapsulates the capsid. The native envelope virus for use herein can be any enveloped virus known in the art or described herein. In certain embodiments, the envelope-virus is selected from the group consisting of: Herpesviruses, Poxviruses (e.g., vaccinia virus), Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus, and Retroviruses.

Also provided herein is a method of making a surface-engineered natively non-enveloped virus, said method comprising: providing a natively non-enveloped virus and applying an artificial coating to the natively non-enveloped virus.

Also provided herein is a composition comprising:

a natively non-enveloped virus; and an artificial coating-layer, wherein the coating-layer encapsulates the natively non-enveloped virus. The natively non-enveloped virus for use herein can be any natively non-enveloped virus known in the art or described herein. In certain embodiments, the natively non-enveloped virus is at least one selected from the group consisting of: adeno-associated virus (including but not limited to AAV2, 3, 5, 6, 8, 9) and adenovirus (including but not limited to Ad5).

Also provided herein is a method of providing a surface coating over a nucleic acid, said method comprising: complexing the nucleic acid with a cation to provide a cationic polyion complex, and applying an artificial coating to the cationic polyion complex.

Also provided herein is a composition comprising:

a nucleic acid contained in a cationic polyion complex; and an artificial coating-layer, wherein the coating-layer encapsulates the nucleic acid contained in the polyion complex. In certain embodiments, the nucleic acid is mRNA, siRNA, miRNA, small nuclear RNA, plasmid DNA, antisense oligodeoxynucleotides, or any other nucleic acid known to those skilled in the art. In certain embodiments, the cation is a cationic polymer such as polyethylenimine or any cationic polymer known to those of skill in the art to be able to generate cationic polyion complexes with nucleic acids. In certain embodiments, the cation is a cationic lipid, such as any cationic lipid known to those of skill in the art to be able to generate cationic complexes with nucleic acids. In certain embodiments, the mRNA encodes patient-specific neoantigens to enable personalized anti-cancer therapy.

In each of the invention coated delivery systems provided herein, the coating-layer protects the viral capsid, non-enveloped virion, or nucleic acid from immune recognition and neutralization during therapy; and/or improve pharmacokinetics in blood/circulation; and/or improves accumulation in target tissue; and/or prevents opsonization and rapid clearance upon systemic administration. In certain embodiments of the invention coated delivery systems provided herein, the coating-layer further comprises binding agents (e.g., targeting-ligands) on its surface that changes the uptake and/or biological activity and/or cell/tissue targeting of the payload in the surface-engineered delivery system relative to the non-coated payload, particularly of the surface-engineered recombinant virus relative to the native envelope-virus.

Also provided herein is a method of making a surface-re-engineered recombinant virus, comprising removing the envelope of an envelope virus to produce a envelope-free-capsid; and encapsulating the envelope-free-capsid with an artificial coating-layer.

Also provided herein are particular aspects of the of the invention, wherein the aspects are numbered below:

1. A replication-competent oncolytic virus comprising:

a first, a second, and a third transgene, whereas each transgene expresses GM-CSF, IL-12, IL-TNF-α, 4-1BBL, CCL4, CCL5/RANTES, CXCL11, or an essential-agent-depletion-enzyme.

2. The replication-competent oncolytic virus of aspect 1, wherein each transgene expresses a different protein.

3. The replication-competent oncolytic virus of aspect 1-2, wherein the first transgene expresses GM-CSF, IL-12, IL-15, TNF-α, or 4-1BBL.

4. The replication-competent oncolytic virus of aspect 1-3, wherein the second transgene expresses CCL4, CCL5/RANTES, CXCL11, 4-1BBL, GM-CSF, or TNF-α.

5. The replication-competent oncolytic virus of aspect 1-4, wherein the third transgene expresses an essential-agent-depletion-enzyme selected from methioninase, adenosine deaminase, or cytosine deaminase, asparaginase or uricase.

6. The replication-competent oncolytic virus of aspect 1-5, wherein the first transgene expresses GM-CSF, IL-12, IL-15, TNF-α, or 4-1BBL; the second transgene expresses CCL4, CCL5/RANTES, CXCL11, 4-1BBL, GM-CSF, or TNF-α; and the third transgene expresses methioninase, adenosine deaminase, or cytosine deaminase.

7. The replication-competent oncolytic virus of aspect 1-6, wherein the third transgene is expressed before the other two transgenes.

8. The replication-competent oncolytic virus of aspect 1-7, wherein the third transgene is expressed under control of an E1 or E3 promoter.

9. The replication-competent oncolytic virus of aspect 1-7, wherein the virus is selected from the group consisting of: adeno-associated virus (AAV), adenovirus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus, Enadenotucirev oncolytic virus, Ad26, Imlygic, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus, Pexa-Vec, Reolysin, DS-1647, TG1042, Cavatak, GL-ONC1, Marabex, ORCA-010, ParvOryx, LOAd703, PV701, MV-NIS, ONCOS-102, Seprehvir, Enadenotucirev, CG0070, Telomelysin, JX-929, VSV Cancer Project, Ad-VirRx 007, NG-348, VSV-GP, RP1/RP2/RP3, WO-12, Maraba virus, Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, Bovine ephemeral fever virus and/or Dimarhabdovirus.

10. The replication-competent oncolytic virus of aspect 1-9, wherein the virus is an adenovirus.

11. The replication-competent oncolytic virus of aspect 10, wherein the adenovirus further comprises a 24-bp deletion in E1A Conserved Region 2 (CR2), and a 2428 bp BsiWI-BglII deletion in the E3 region.

12. The replication-competent oncolytic virus of aspect 1-11, wherein the first, second, and third transgenes are selected from the group of first, second and third transgene combinations set forth in Table 1 and FIGS. 19-21.

13. The replication-competent oncolytic virus of aspect 1-12, wherein the first, second, and third transgenes are selected from the group of first, second and third transgene combinations selected from:

| | | | |
|---|---|---|---|
| Ad5.DEV09 | RFP | hCCL5/RANTES-human | IL15-human |
| Ad5.DEV10 | Adenosine Deaminase-human | hCCL5/RANTES-human | IL15-human |
| Ad5.DEV11 | Methioninase | hCCL5/RANTES-human | IL15-human |
| Ad5.DEV12 | Methioninase | Adenosine Deaminase-human | IL 15-human |
| Ad5.DEV13 | Methioninase | Adenosine Deaminase-human | GM-CSF-mouse |
| Ad5.DEV14 | Adenosine Deaminase-human | GM-CSF-mouse | IL15-human |
| Ad5.DEV15 | Methioninase | GM-CSF-mouse | IL15-human |

14. A replication-competent oncolytic virus comprising:

a first, and a second transgene, whereas each transgene expresses GM-CSF, IL-12, IL-15, TNF-α, 4-1BBL, CCL4, CCL5/RANTES, CXCL11, or an essential-agent-depletion-enzyme, wherein each transgene expresses a different protein, and wherein the first and second transgenes are selected from the group of first and second transgene combinations set forth in Table 1 and FIGS. 19-21.

15. A method of treating cancer in an individual in need thereof, comprising administering to the individual a replication-competent oncolytic virus of aspects 1-14, or a surface-engineered recombinant oncolytic virus; thereby treating the individual.

16. The method of aspect 15, wherein the surface-engineered recombinant oncolytic virus comprises the replication-competent oncolytic virus of aspects 1-14.

17. The method of aspects 15-16, wherein the replication-competent oncolytic virus or surface-engineered recombinant oncolytic virus is administered in combination with or as an adjuvant to another anti-cancer drug.

18. The method of aspects 15-17, wherein the replication-competent oncolytic virus or surface-engineered recombinant oncolytic virus is administered once as a single dose, or repeatedly at 2 or more intervals.

19. A method of administering a gene therapy to an individual in need thereof, comprising administering to the individual a surface-engineered recombinant gene-therapy virus comprising a transgene encoding a therapeutic protein; thereby administering the gene therapy to the individual.

The method of aspect 19, wherein the surface-engineered recombinant gene-therapy virus is administered by intramuscular, intravenous, intracranial, or intrathecal injection, or injection into any tissue where transgene expression is desired.

21. The method of aspects 19-20, wherein the surface-engineered recombinant gene-therapy virus is administered once as a single dose, or repeatedly at 2 or more intervals.

22. A surface-engineered recombinant virus, said virus comprising:
a recombinant virus having a recombinant genome, or a replication-competent oncolytic virus of aspects 1-14; and
an artificial coating layer surrounding the recombinant virus.

23. The surface-engineered recombinant virus of aspect 22, wherein the artificial coating is selected from the group consisting of: silica, titanium oxide and calcium phosphate.

24. The surface-engineered recombinant virus of aspects 22-23, wherein the virus is selected from the group consisting of: enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, and hepatitis A virus.

25. The surface-engineered recombinant virus of aspects 22-24, wherein the recombinant virus is oncolytic and is selected from the group consisting of: NG-641 (PsiOxus), and Imlygic (talimogene laherparepvec).

26. The surface-engineered recombinant virus of aspects 22-24, wherein the virus is a vaccine and is selected from AZD1222 (AstraZeneca), ChAdOx1-nCov19 (Oxford), Ad5-nCoV (CanSino), VSV, and Ad26 (J&J).

27. The surface-engineered recombinant virus of aspect 22, wherein the virus is replication deficient Ad5 (Human) Adenovirus vector, and wherein the recombinant genome encodes SARS-CoV-2 spike protein and E1 & E3 genes are deleted; or wherein the adenovirus further comprises a 24-bp deletion in E1A Conserved Region 2 (CR2), and a 2428 bp BsiWI-BglII deletion in the E3 region; or wherein the virus does not contain any native Ad5 viral genes.

28. The surface-engineered recombinant virus of aspect 22, wherein the virus is oncolytic and the virus is VSV.

29. The surface-engineered recombinant virus of aspects 22-28, wherein the artificial coating is applied by conducting a charge-mediated sol-gel condensation reaction directly onto the surface of the recombinant virus.

30. The surface-engineered virus of aspects 22-29, wherein the artificial coating comprises a silica gel matrix or titanium oxide gel matrix encapsulating the recombinant virus.

31. The surface-engineered virus of aspects 22-30, wherein the artificial coating comprises a targeting-ligand selected from the group consisting of: proteins, polysaccharides, aptamers, peptides, oligonucleotides and small molecules.

32. The surface-engineered virus of aspects 22-31, wherein the artificial coating comprises a targeting-ligand selected from the group consisting of: Antibodies, transferrin, Hyaluronic acid, RGD, IL4RPep-1, AS-1411, GBI-10, Folate, anisamide, and phenylboronic acid.

33. The surface-engineered virus of aspects 22-32, wherein the recombinant virus is replication-competent or replication-defective.

34. The surface-engineered virus of aspects 22-33, wherein the recombinant virus has had it native-envelope removed prior to coating with the artificial coating layer.

35. A surface-engineered recombinant virus vaccine, said virus comprising:
a recombinant Ad5 virus having a recombinant genome encoding SARS-CoV-2 spike protein, wherein at least a functional portion of E1 and E3 genes are deleted, or wherein the adenovirus further comprises a 24-bp deletion in E1A Conserved Region 2 (CR2), and a 2428 bp BsiWI-BglII deletion in the E3 region; or wherein the virus does not contain any native Ad5 viral genes;
a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating layer encapsulating the recombinant Ad5 virus, wherein said coating layer comprises an effective amount of folate to bind a folate receptor on a cell, and wherein the artificial coating is selected from the group consisting of: silica, titanium oxide and calcium phosphate.

36. A method of making a surface-engineered recombinant virus, said method comprising:
producing a recombinant virus having a recombinant genome, or a replication-competent oncolytic virus of aspects 1-14; and
applying an artificial coating to the recombinant virus, wherein the artificial coating is selected from the group consisting of: silica, titanium oxide and calcium phosphate.

37. The method of aspect 36, wherein the artificial coating is applied by conducting a charge-mediated sol-gel condensation reaction directly onto the surface of the recombinant virus.

38. The method of aspects 36-37, wherein the artificial coating comprises a silica gel matrix or titanium oxide gel matrix encapsulating the recombinant virus.

39. The method of aspects 36-38, wherein the recombinant virus has had its native-envelope removed prior to coating with the artificial coating layer.

40. A method of re-engineering the surface of a virus having a native-envelope, said method comprising:
removing the native-envelope from the virus to isolate a previously-enveloped-capsid;
applying an artificial coating to the previously-enveloped-capsid, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme.

41. The method of aspect 40, wherein the native-envelope virus is selected from the group consisting of: Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

42. The method of aspects 40-41, wherein the native-envelope is removed or delipidated using a detergent and/or an extraction solvent.

43. The method of aspect 42, wherein the detergent and/or an extraction solvent is selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination.

44. The method of aspects 40-43, wherein the artificial coating is selected from the group consisting of: silica, titanium oxide and calcium phosphate.

45. The method of aspects 40-44, wherein applying the artificial coating further comprises conducting a charge-mediated sol-gel condensation reaction directly onto the surface of the previously-enveloped-capsid.

46. The method of aspects 40-45, wherein the artificial coating comprises a silica gel matrix or titanium oxide gel matrix encapsulating the previously-enveloped-capsid.

47. The method of aspects 45-46, wherein the previously-enveloped-capsid is replication-competent or replication-defective.

48. A surface-re-engineered virus comprising:
a previously-enveloped-capsid from a naturally occurring enveloped-virus, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating layer surrounding the previously-enveloped-capsid.

49. The surface-re-engineered virus of aspect 48, wherein the envelope virus is selected from the group consisting of: Poxvirus, vaccinia virus, Herpes Virus, herpes simplex virus-1, herpes simplex virus-2, Rhabdovirus, Vesicular stomatitis virus (VSV), Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

50. The surface-re-engineered virus of aspects 48-49, wherein the artificial coating is selected from the group consisting of: silica, titanium oxide and calcium phosphate.

51. The surface-re-engineered virus of aspects 48-50, wherein the artificial coating is applied by conducting a charge-mediated sol-gel condensation reaction directly onto the surface of the previously-enveloped-capsid,

52. The surface-re-engineered virus of aspects 48-51, wherein the artificial coating comprises a silica gel matrix or titanium oxide gel matrix encapsulating the previously-enveloped-capsid.

53. The surface-re-engineered virus of aspects 48-52, wherein the previously-enveloped-capsid is replication-competent or replication-defective.

54. A method of re-engineering a virus having a native-envelope, said method comprising:
removing the native-envelope surrounding a capsid from the virus, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme;
isolating the previously-enveloped-capsid; and
applying an artificial coating to the previously-enveloped-capsid.

55. A composition comprising;
a capsid from a native envelope-virus, wherein the capsid is devoid of its native envelope, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating-layer, wherein the coating-layer encapsulates the capsid.

56. The composition of aspect 55, wherein the envelope-virus is selected from the group consisting of: Herpesviruses, Poxviruses (e.g., vaccinia virus), Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus, and Retroviruses.

57. The composition of aspects 55-56, wherein the coating-layer protects the capsid from immune recognition and neutralization during therapy.

58. The composition of aspects 55-57, wherein the coating-layer further comprises binding agents on its surface that changes the infectivity and/or biological activity of the native envelope-virus.

59. A method of making the composition of aspects 55-58, comprising removing the envelope of an envelope virus to produce an envelope-free-capsid; and encapsulating the envelope-free-capsid with an artificial coating-layer.

60. A surface-engineered delivery system, said system comprising:
a payload, or a replication-competent oncolytic virus of aspects 1-14; and
an artificial coating layer surrounding the payload.

61. The surface-engineered delivery system of clam 60, wherein the payload is selected from a recombinant virus or a nucleic acid.

62. The surface-engineered delivery system of aspects 60-61, wherein the artificial coating is selected from the group consisting of: silica, titanium oxide and calcium phosphate.

63. The surface-engineered recombinant virus of aspect 27, wherein the coating of the virus increases humoral immunity induced by the virus against SARS-CoV-2 spike protein.

64. The surface-engineered recombinant virus of aspect 63, wherein the increased humoral immunity is characterized by increased IgG antibody levels.

65. The surface-engineered recombinant virus of aspect 27, wherein the coating of the virus increases cellular immunity induced by the virus against SARS-CoV-2 spike protein.

66. The surface-engineered recombinant virus of aspect 65, wherein the increased cellular immune is characterized by increased CD8+ lymphocyte levels.

67. The surface-engineered recombinant virus of aspect 65, wherein the increased cellular immunity is characterized by increased memory T cell levels.

68. The surface-engineered recombinant virus vaccine of aspect 35, wherein the coating of the virus increases humoral immunity induced by the virus against SARS-CoV-2 spike protein.

69. The surface-engineered recombinant virus vaccines of aspect 68, wherein the increased humoral immunity is characterized by increased IgG antibody levels.

70. The surface-engineered recombinant virus vaccine of aspect 35, wherein the coating of the virus increases cellular immunity induced by the virus against SARS-CoV-2 spike protein.

71. The surface-engineered recombinant virus of aspect 70, wherein the increased cellular immune is characterized by increased CD8+ lymphocyte levels or activity.

72. The surface-engineered recombinant virus of aspect 70, wherein the increased cellular immunity is characterized by increased memory T cell levels or activity.

73. The surface-engineered recombinant virus of aspect 27, wherein the coating of the virus increases immunity induced by the virus against SARS-CoV-2 spike protein in an individual having pre-existing neutralizing antibodies against Ad5 adenovirus.

74. The surface-engineered recombinant virus of aspect 73, wherein the increased immunity is characterized by increased IgG antibody levels.

75. The surface-engineered recombinant virus of aspect 73, wherein the increased immunity is characterized by increased CD8+ lymphocyte levels or activity.

76. The surface-engineered recombinant virus of aspect 73, wherein the increased immunity is characterized by increased memory levels or activity.

### BRIEF DESCRIPTION OF THE FIGURES

FIG. 1 shows the process for generating the inventive surface-engineered recombinant virus, where a coating is generated over a recombinant virus by first applying an organic coating and then inducing a sol-gel reaction. Once the surface of the virus has been engineered by coating with the sol-gel, ligands and other functional molecules are attached to the surface using PEG spacers to provide new functionality. The figure shows coating of adenovirus, but this method is applicable to all viruses. For enveloped viruses, a step can be applied to strip the virus of its envelop before applying the coating.

FIG. 2 shows electron microscope images of surface-engineered vaccinia virus and adenovirus generated using the inventive coating. The coating utilizes inorganic/organic hybrid chemistry, with one-step synthesis at room temperature and neutral pH with a ˜4 hour reaction duration. This enables very flexible surface functionalization chemistry that allows high-density attachment of small molecules, peptides, oligos, proteins, sugars or other small/large molecules. The coating provides 10-100 fold improvement of cancer cell infectivity and 10-100 fold improvement of blood circulation half life. The coating is characterized by optimized and scalable chemistry with great batch-to-batch consistency, and can be applied to both enveloped and non-enveloped viruses. The coating has thus far been applied to Vaccinia Virus, Adenovirus, Measles Virus, Herpes Simplex Virus, Retroviral Replicating Virus, and Vesicular Stomatitis Virus, and is thus is easily applicable to any virus.

FIG. 3 shows a schematic representation of the benefits provided by the surface coating on the surface-engineered recombinant virus in vivo. The coating prevents surface access of antibodies to prevent neutralization of the virus, thus allowing systemic administration and repeated administration. The coating reduces liver uptake and prevents clearance by the reticuloendothelial system, thus prolonging blood circulation. The coating allows attachment of targeting molecules to achieve tissue targeting.

FIG. 4 shows the scheme for large-scale GMP-grade manufacturing of the surface-engineered recombinant virus. The coating reaction is a bulk a bulk reaction performed at 25° C., and the total reaction takes 1 hour. The reaction is performed at neutral pH in aqueous conditions. It is a simple one-step reaction easily implemented during the last stage of viral vector production. The coating is self-assembling, and the process is self-terminating without any other reagents required.

FIG. 5 shows the uptake and delivery mechanism for the surface-engineered recombinant virus when folate is attached as an exemplary targeting molecule. The folate attached to the surface of the surface-engineered virus binds to the folate receptor (which is highly present on cancer cells), stimulating receptor-mediated endocytosis of the surface-engineered recombinant virus. The coating degrades in the endosome, resulting in acidification of the endosome that triggers endosomolysis, releasing the virus into the cytosol. The now-uncoated naked virus is fully functional and now behaves the same as the native virus upon viral entry into the cell; it inserts its genome into the cell nucleus and replicates. Eventually, the viral replication causes the cell to lyse and release the newly produced viral particles. Other receptors and other uptake mechanisms can be utilized depending on the nature of the ligand attached to the surface-engineered recombinant virus.

FIG. 6 shows a comparison of uptake mechanisms between the surface-engineered recombinant virus and native enveloped and non-enveloped viruses. When a ligand molecule is attached to its surface, the surface-engineered recombinant virus utilizes receptor-mediated endocytosis similarly to a non-enveloped virus. By changing the ligand, the cell/tissue tropism of the surface-engineered recombinant virus can be changed. Functional molecules can also be attached to the surface-engineered recombinant virus to enhance endosomal rupture/escape or nuclear transport and entry.

FIG. 7 shows that the inventive coating does not negatively affect the infectivity of the virus; reporter protein expression at 4 days after infection in vitro was similar between naked and coated vesicular stomatitis virus.

FIG. 8 shows that the inventive coating increases viral infectivity, with reporter protein activity after in vitro Ad5 infection increased by nearly two fold.

FIG. 9 shows that the inventive coating protects the virus from antibody neutralization, with reporter protein expression maintained after Ad5 virus infection even in the presence of antibody concentrations that completely neutralized non-coated Ad5 virus.

FIGS. 10A and 10B show the immune stealth property conferred by the inventive coating. The coating reduced the in vitro macrophage response to Ad5 virus, even out to 80 days, as measured by macrophage GM-CSF and IL-1α secretion. This indicates that the coating reduced the recognition of the virus by macrophages.

FIGS. 11A and 11B show that attaching a ligand to the surface-engineered recombinant virus allows cell-specific targeting. Specifically, tethering of folate to the surface of the surface-engineered recombinant Ad5 virus resulted in increased reporter gene expression in U2OS cells with high folate receptor expression and reduced reporter gene expression in H226 cells with low folate receptor expression when compared to non-coated Ad5 virus. The increased expression was due to the presence of folate on the surface of the surface-engineered virus, whereas the reduced expression was due to the coating and PEG layer preventing nonspecific cell entry.

FIGS. 12A and 12B show that the coating increases the in vivo circulation time for the surface-engineered recombinant virus. The coating increased the circulation time for adenovirus compared to non-coated adenovirus after tail vein injection in mice, in both naïve mice and mice that had been previously immunized with a live virus. The coating thus prevented the adenovirus from being cleared from the circulation, even after previous immunization.

FIG. 12C shows that the coating allows repeated administration of the surface-engineered recombinant virus. Repeated immunization of mice with a coated Ad5 vaccine, with three intramuscular administrations one week apart, resulted in further increases in serum neutralizing antibody titer with each successive injection.

FIGS. 13A and 13C show that the coating allows anti-tumor treatment at lower doses of the surface-engineered recombinant virus in vivo. In tumor-implanted immunodeficient mice receiving an anti-tumor vaccinia virus, coating of the virus still reduced tumor size at only 30% of the conventional dose, and the tumor size reduction at the lower dose was greater than that observed with the non-coated virus.

FIGS. 13B and 13D show that the coating allows anti-tumor treatment at lower doses of the surface-engineered recombinant virus in vivo. In tumor-implanted immunodeficient mice receiving an anti-tumor vaccinia virus, coating of the virus still increased survival at only 30% of the conventional dose, and the survival benefit at the lower dose was greater than that observed with the non-coated virus.

FIG. 14A shows that coating of a vaccine results in stronger cellular immunity. Coating of Ad5 virus expressing the COVID-19 spike protein resulted in increased IgG antibody generation in mice, even out to day 79, after three intramuscular injections, two weeks apart.

FIG. 14B shows that coating of a vaccine results in stronger cellular immunity. Coating of Ad5 virus expressing the COVID-19 spike protein resulted in increased CD8+ lymphocyte levels in mice after 3 intramuscular injections, two weeks apart.

FIG. 15 shows that coating of a protein antigen as the payload results in prolonged tissue residence, even out to day 45.

FIG. 16 shows that coating of a virus produces a stronger anti-tumor effect in vivo compared to non-coated virus when the virus is injected at a suboptimal dose. To generate a syngeneic, immunocompetent mouse model of breast cancer, immunocompetent mice were injected with 4T1 mammary carcinoma cells. At 9 days after tumor cell inoculation, the mice were injected with 108 pfu/mouse ONCoat-Ad5, naked Ad5, or saline as a control. ONCoat-Ad5, but not naked Ad5, reduced tumor growth compared to the saline control out to at least 20 days after tumor inoculation, as indicated by delayed increase of tumor volume. The dose used was orders of magnitude lower than would normally be used for naked Ad5; thus, ONCoat achieved tumor retardation with orders of magnitude suboptimal dose in a non-permissive mouse model. Thus, immunocompetent tumor-implanted mice injected with coated Ad5 virus showed reduced tumor growth compared to mice injected with non-coated Ad5 virus.

FIG. 17 shows effective induction of humoral immunity (i.e., antibody generation) against SARS-CoV-2 spike protein in vivo. Coating of the virus allows effective antibody generation in vivo even in the presence of neutralizing antibodies against Ad5, i.e., when there has been previous exposure to Ad5.

FIG. 18 shows effective induction of cellular immunity (i.e., interferon gamma secretion from T cells). This indicates induction of cellular immunity mediated by CD8+ T cells and/or memory T cells. Coating of the virus allows effective antibody generation in vivo even in the presence of neutralizing antibodies against Ad5, i.e., when there has been previous exposure to Ad5.

FIG. 19 shows a tabular description of various particular embodiments of invention replication-competent oncolytic viruses.

FIG. 20A shows a tabular description of various particular embodiments of invention replication-competent oncolytic viruses, including their respective surface engineering, replication selectivity, and transgenes.

FIG. 20B shows a tabular description of various particular embodiments of invention replication-competent oncolytic viruses, including their respective surface engineering, replication selectivity, and transgenes.

FIG. 21A shows a tabular description of various particular embodiments of invention replication-competent oncolytic viruses, including their respective transgenes.

FIG. 21B shows a tabular description of various particular embodiments of invention replication-competent oncolytic viruses, including their respective transgenes.

FIG. 22 shows a tabular description of various particular embodiments of invention replication-competent oncolytic viruses, including their respective transgenes and the respective promoter driving each transgene, as well as a schematic representation of the Ad5 viral backbone showing where each transgene is inserted and also showing the 24-bp deletion in E1A Conserved Region 2.

FIG. 23 shows the construction of the pAd1129-RFP, pAd1129-ADA and pAd1129-Met shuttle plasmids used to insert the red fluorescent protein, adenosine deaminase, and methioninase transgenes in Ad vectors.

FIG. 24 shows the construction of the pAd1149-mIL12, pAd1149-hIL15 and pAd1149-mGMCSF shuttle plasmids used to insert the IL-12, IL-15, and GM-CSF transgenes between the L5 and E4 polyA signals in Ad vectors under control of a CMV promoter and SV40pA signal.

FIG. 25 shows the construction of the pAd1148-ADA, pAd1148-Met, pAd1148-hIL15, pAd1148-mGMCSF and pAd1148-hCCL5 shuttle plasmids used to insert the adenosine deaminase, methioninase, IL-15, GM-CSF, and CCL-5 transgenes between the L5 and E4 polyA signals in Ad vectors under control of a RSV promoter and bGHpA signal.

### DETAILED DESCRIPTION

Provided herein is a replication-competent oncolytic virus comprising a replication-competent oncolytic virus comprising: a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme. In particular embodiments, each transgene expresses a different protein. In other embodiments, the first transgene expresses GM-CSF, IL-12, IL-15, TNF-α, or 4-1BBL. In yet another embodiment, the second transgene expresses CCL4, CCL5/RANTES, CXCL11, 4-1BBL, GM-CSF, or TNF-α. In another embodiment, the third transgene expresses an essential-agent-depletion-enzyme selected from methioninase, adenosine deaminase, or cytosine deaminase, asparaginase or uricase. In a particular embodiment, the first transgene expresses GM-CSF, IL-12, IL-15, TNF-α, or 4-1BBL; the second transgene expresses CCL4, CCL5/RANTES, CXCL11, 4-1BBL, GM-CSF, or TNF-α; and the third transgene expresses methioninase, adenosine deaminase, or cytosine deaminase. In a particular embodiment, the third transgene is expressed before the other two transgenes. In further embodiments, the third transgene is expressed under control of an E1 or E3 promoter.

In certain embodiments, the virus is selected from the group consisting of: adeno-associated virus (AAV), adenovirus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus, Enadenotucirev oncolytic virus, Ad26, Imlygic, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus, Pexa-Vec, Reolysin, DS-1647, TG1042, Cavatak, GL-ONC1, Marabex, ORCA-010, ParvOryx, LOAd703, PV701, MV-NIS, ONCOS-102, Seprehvir, Enadenotucirev, CG0070, Telomelysin, JX-929, VSV Cancer Project, Ad-VirRx 007, NG-348, VSV-GP, RP1/RP2/RP3, WO-12, Maraba virus, Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, Bovine ephemeral fever virus and/or Dimarhabdovirus.

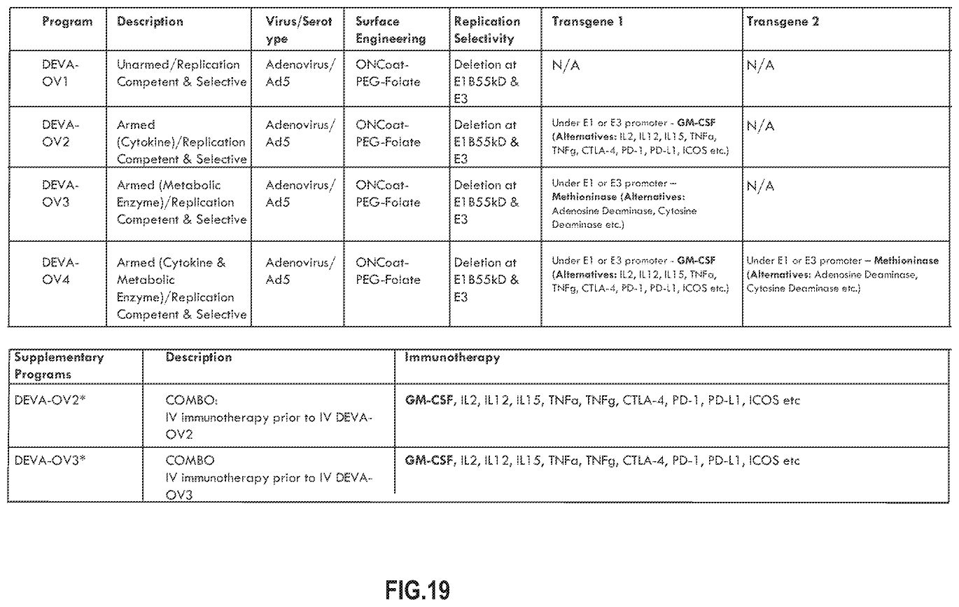

In a particular embodiment, the virus is an adenovirus. In yet another embodiment, the adenovirus further comprises a 24-bp deletion in E1A Conserved Region 2 (CR2), and a 2428 bp BsiWI-BglII deletion in the E3 region. In particular embodiments, the first, second, and third transgenes are selected from the group of first, second and third transgene combinations set forth in Table 1 and FIGS. 19-21. In another embodiment, the first, second, and third transgenes are selected from the group of first, second and third transgene combinations selected from:

| | | | |
|---|---|---|---|
| Ad5.DEV09 | RFP | hCCL5/RANTES-human | IL15-human |
| Ad5.DEV10 | Adenosine Deaminase-human | hCCL5/RANTES-human | IL15-human |
| Ad5.DEV11 | Methioninase | hCCL5/RANTES-human | IL15-human |
| Ad5.DEV12 | Methioninase | Adenosine Deaminase-human | IL15-human |
| Ad5.DEV13 | Methioninase | Adenosine Deaminase-human | GM-CSF-mouse |
| Ad5.DEV14 | Adenosine Deaminase-human | GM-CSF-mouse | IL15-human |
| Ad5.DEV15 | Methioninase | GM-CSF-mouse | IL15-human |

In another embodiment, provided herein is a replication-competent oncolytic virus comprising a first, and a second transgene, whereas each transgene expresses GM-CSF, IL-12, IL-15, TNF-α, 4-1BBL, CCL4, CCL5/RANTES, CXCL11, or an essential-agent-depletion-enzyme, wherein each transgene expresses a different protein, and wherein the first and second transgenes are selected from the group of first and second transgene combinations set forth in Table 1 and FIGS. 19-21.

As used herein, the phrase “oncolytic virus” refers to a virus that preferentially infects and kills cancer cells. As the infected cancer cells are destroyed by oncolysis, they release new infectious virus particles or virions to help destroy the remaining tumor or tumors.

In other words, the invention recombinant viruses provided herein can comprise combinations of at least 2 (e.g., a first transgene and a second transgene, or grammatical variations thereof), or combinations of at least 3 transgenes (e.g., a first transgene, a second transgene and a third transgene combination, or grammatical variations thereof) are set forth in each respective row in Table 1; or that can be combined in any of the genomes of an virus set forth herein, e.g., in Ad5, herpes virus, enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, and hepatitis A virus. In other embodiments, the surface-engineered recombinant virus is an oncolytic virus and is selected from the group consisting of: NG-641 (PsiOxus), and Imlygic (talimogene laherparepvec), and the like. In particular embodiments, the following combinations of cytokines, chemokines, and/or enzymes set forth in Table 1 are delivered by being encoded in the recombinant virus genome or nucleic acids, by being carried in surface-engineered delivery systems, or otherwise as described above:

TABLE 1

| Transgene 1 | Transgene 2 | Transgene 3 |
|---|---|---|
| GM-CSF/IL-12/ | CCL4/CCL5/CXCL11 | Methioninase/adenosine |
| IL-15/TNF-α/4- | | deaminase/cytosine |
| 1BBL | | deaminase |
| IL-12 | 4-1BBL | Adenosine deaminase |
| GM-CSF | CCL4 | Adenosine deaminase |
| IL-15 | CCL5 | Adenosine deaminase |
| IL-15 | CCL4 | Methioninase |
| IL-15 | TNF-α | Adenosine deaminase |
| IL-15 | CCL4 | Adenosine deaminase |
| IL-15 | CCL4 | Methioninase |
| IL-15 | CCL5 | Adenosine deaminase |
| IL-15 | CCL5 | Methioninase |
| IL-15 | None | None |
| IL-15 | CCL5 | None |
| Adenosine deaminase | None | None |
| Methioninase | None | None |
| IL-15 | Adenosine deaminase | Methioninase |
| IL-15 and/or IL-12 | GM-CSF | None |
| IL-15 and/or IL-12 | GM-CSF | Adenosine deaminase |
| IL-15 and/or IL-12 | GM-CSF | Methioninase |
| GM-CSF | Adenosine deaminase | Methioninase |
| IL-15 and/or IL-12 | None | None |
| IL-12 | Adenosine deaminase | None |
| IL-12 | Methioninase | None |
| IL-12 | IL-15 | None |
| IL-15 | CCL5 and/or RANTES | Adenosine deaminase |
| IL-15 | CCL5 and/or RANTES | Methioninase |
| IL-12 | Anti-PD-L1 | None |
| IL-15 | Anti-PD-L1 | None |
| IL-15 | Anti-PD-L1 | Adenosine deaminase |
| IL-15 | Anti-PD-L1 | Methioninase |
| IL-15 | CCL5 and/or RANTES | None |
| Anti-PD-L1 | IL-15 | None |
| Anti-PD-L1 | IL-15 | Adenosine deaminase |
| Anti-PD-L1 | IL-15 | Methioninase |
| Anti-PD-L1 | IL-12 | None |
| Anti-PD-L1 | GM-CSF | None |
| Anti-PD-L1 | GM-CSF | Adenosine deaminase |
| Anti-PD-L1 | GM-CSF | Methioninase |
| Anti-PD-L1 | IL-15 | GM-CSF |
| IL-12 | Adenosine deaminase | Adenosine deaminase |
| IL-12 | CCL and/or RANTES | None |
| IL-12 | CCL and/or RANTES | Adenosine deaminase |
| IL-12 | CCL and/or RANTES | Methioninase |

Delivery of the abovementioned cytokines, chemokines, enzymes, and combinations thereof by being encoded in the recombinant virus genomes or nucleic acids, by being carried in surface-engineered delivery systems, or otherwise as described above can provide various therapeutic benefits. Such therapeutic benefits include proliferation, activation, and survival of CD8+, T, NK, NKT, and dendritic cells; promotion of memory T cells; and maintenance or improvement of vitality of amplification of cytotoxic T lymphocytes. Such therapeutic benefits include improving antigen presentation through recruitment and activation of dendritic cells and macrophages. Such therapeutic benefits include prevention of immunosuppression and maintenance or improvement of T cell infiltration. Such therapeutic benefits include enhancement of viral replication for the delivered recombinant virus and expansion of antitumor efficacy. Such therapeutic benefits include T cell activation and proliferation, and differentiation of T cells into Th1 cells. Such therapeutic benefits include prevention of T cell suppression and prolongation of T cell engagement at the tumor site, enhancing anti-tumor immunity. Such therapeutic benefits include selective apoptosis in cancer cells, expanding the anti-tumor effect to the entire tumor microenvironment. Such therapeutic benefits include stimulation of dendritic cells and direct recruitment of natural killer cells. Such therapeutic benefits include proliferation of both CTL and NK cells.

In some embodiments, where the recombinant virus genome is an adenovirus genome encoding any of the chemokines, cytokines, enzymes, and combinations described above. In some embodiments, the adenovirus genome also contains deletions. In some embodiments, the E1A gene is deleted. In some embodiments, a portion of the E1A gene, such as a 24-bp portion, is deleted. In some embodiments, the E3 gene or a portion thereof is deleted. In some embodiments, the E1A gene or a portion thereof and the E3 gene or a portion thereof are both deleted. Such deletions, including the E1A-24 bp/E3 deletion, make it so that the recombinant virus selectively replicates in cancer cells, thus allowing the activity and/or transgene expression of the virus to be targeted to cancer cells. Deletion at E1 B55KD prevents disruption of the p53 pathway. Deletion of E3 enhances the immune response.

In an embodiment, the surface-engineered virus is replication-competent and selective, without carrying a transgene. In this embodiment, the virus is Ad5, carrying a deletion at E1B55 kD and E3 for replication selectivity. The surface engineering is achieved by application of ONCoat, followed by surface functionalization with PEG and folate. The surface-engineered virus can be administered alone or in combination with or as an adjunct to an intravenous immunotherapy administered prior to the surface-engineered virus.

In an embodiment, the surface-engineered virus is replication-competent and selective, and carrying a cytokine-expressing transgene. In this embodiment, the virus is Ad5, carrying a deletion at E1B55 kD and E3 for replication selectivity. The surface engineering is achieved by application of ONCoat, followed by surface functionalization with PEG and folate. The transgene expresses GM-CSF under control of the E1 or E3 promoter. Alternatively, the transgene may express IL2, IL12, IL15, TNF alpha, TNF gamma, or another cytokine. Alternatively, rather than a cytokine, the transgene may express other molecules including immune checkpoint-related molecules such as CTLA-4, PD-1, PD-L1, or ICOS. The surface-engineered virus can be administered alone or in combination with or as an adjunct to an intravenous immunotherapy administered prior to the surface-engineered virus.

In an embodiment, the surface-engineered virus is replication-competent and selective, and carrying a metabolic enzyme-expressing transgene. In this embodiment, the virus is Ad5, carrying a deletion at E1B55 kD and E3 for replication selectivity. The surface engineering is achieved by application of ONCoat, followed by surface functionalization with PEG and folate. The transgene expresses methioninase under control of the E1 or E3 promoter. Alternatively, the transgene may express adenosine deaminase or cytosine deaminase. The surface-engineered virus can be administered alone or in combination with or as an adjunct to an intravenous immunotherapy administered prior to the surface-engineered virus.

In an embodiment, the surface-engineered virus is replication-competent and selective, and carrying two transgenes: a cytokine-expressing transgene and a metabolic enzyme-expressing transgene. In this embodiment, the virus is Ad5, carrying a deletion at E1B55 kD and E3 for replication selectivity. The surface engineering is achieved by application of ONCoat, followed by surface functionalization with PEG and folate. The first transgene expresses GM-CSF under control of the E1 or E3 promoter. Alternatively, the first transgene may express IL2, IL12, IL15, TNF alpha, TNF gamma, or another cytokine. Alternatively, rather than a cytokine, the first transgene may express other molecules including immune checkpoint-related molecules such as CTLA-4, PD-1, PD-L1, or ICOS. The second transgene expresses methioninase under control of the E1 or E3 promoter. Alternatively, the second transgene may express adenosine deaminase or cytosine deaminase. The surface-engineered virus can be administered alone or in combination with or as an adjunct to an intravenous immunotherapy administered prior to the surface-engineered virus.

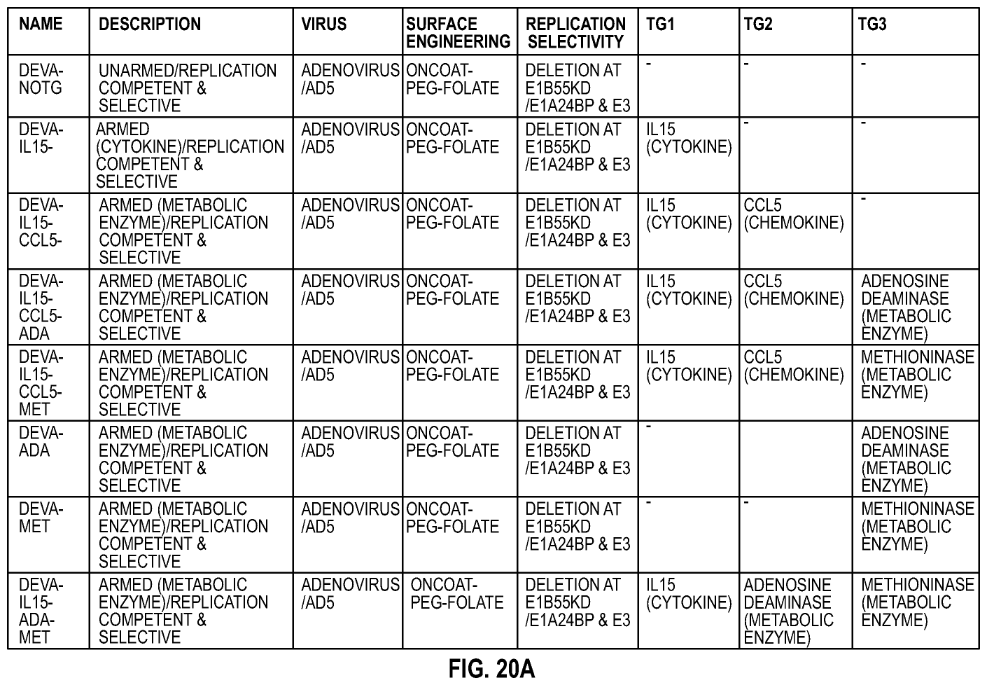

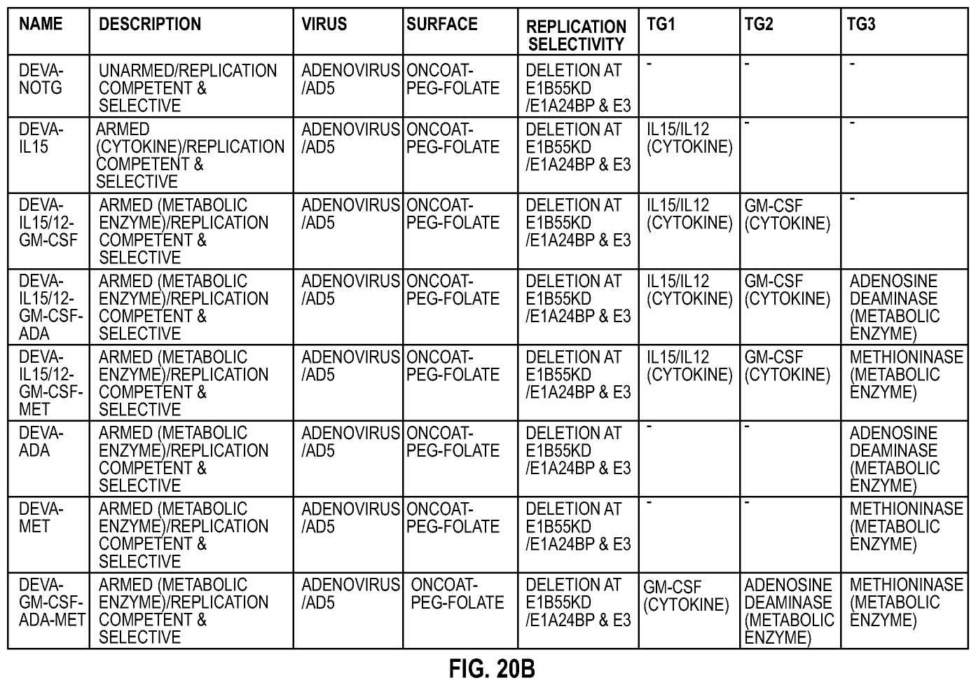

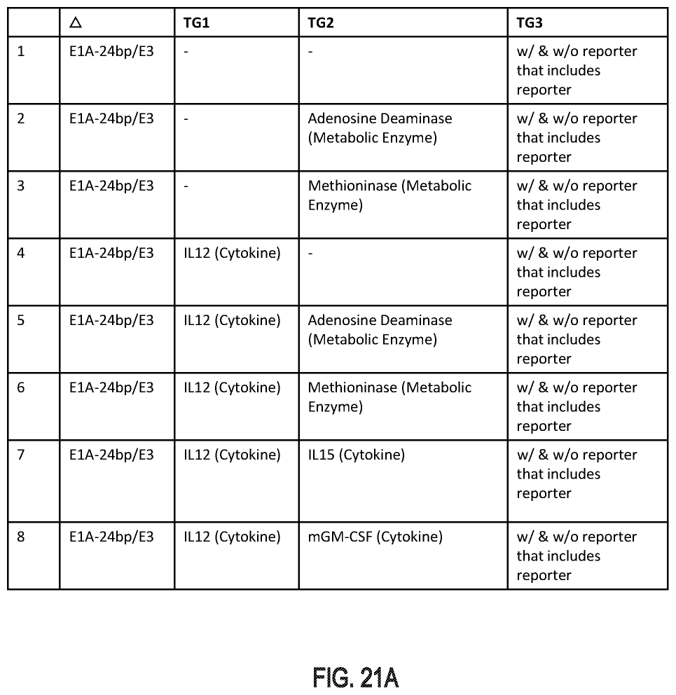

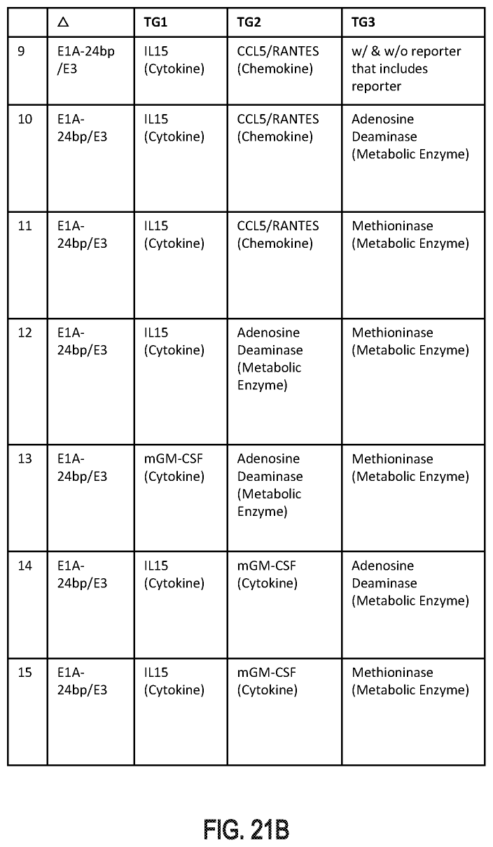

In the embodiments herein, the components of the surface-engineered virus offer various benefits. The ONCoat coating on the virus provides stable encapsulation, easy functionalization, and high efficient pH-dependent cytoplasmic release of functional and unhampered virus within cancer cells. The heavy PEGylation on the surface provides a stealth coating, preventing neutralization and opsonization, thereby improving PK and accumulation in main tumor and metastases. The use of folate as a ligand provides enhanced tumor tissue retention and cancer cell uptake as folate receptors are over-expressed in most cancers. Because folate receptors have some level of expression in many cell types, the use of folate as a ligand also can provide for widespread, systemic delivery, and it can also allow delivery to various cell types, i.e., any cell type expressing the folate receptor. Selective targeting to cells over-expressing the folate receptor or general delivery to any cell expressing the folate receptor can be determined by the dose of the surface-engineered virus administered. The use of Ad5 as the virus is beneficial because Ad5 is a common human pathogen with broad tropism and high infectivity, high immunogenicity, small size, good safety, and established manufacturing and engineering processes. The deletions in the Ad5 genome confer replication selectivity; the deletion at E1B55KD prevents interference with the p53 pathway), whereas the deletion at E3 enhances the immune response. Expression of cytokines from transgenes, particularly driven by the E3 promoter, provides the following effects. GM-CSF stimulates dendritic cells and direct recruitment of natural killer cells. IL-2 stimulates of T-cell proliferation and differentiation. IL-12 enhances the proliferation of both CTL and NK cells. Expression of metabolic enzymes from transgenes, particularly driven by the E1 promoter, provides the following effects in accordance with the present invention. Methioninase arrests cells at the G2/S phase to enhance viral replication and selectively trigger apoptosis in tumor cells not infected by the virus (i.e., bystander effect). Adenosine deaminase depletes adenosine from the tumor tissue to stop migration of T-cells of the tumor tissue. The transgenes described above can be combined, with one, two, or three transgenes present in a single virus to provide combinations of the effects described above. Such combinations are shown in FIG. 20A and FIG. 20B; and FIG. 21A and FIG. 21B. Including methioninase as the first transgene under control of the early promoter is believed to work better than other configurations because it permits the highest amount of viral replication by arresting the host cell in Si phase through depletion of methionine.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgene encodes adenosine deaminase and is in the second transgene position, with no transgenes in the first or third transgene positions. This virus is believed to prevent immune suppression and prolong T cell engagement at the tumor site, enhancing anti-tumor immunity.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgene encodes methioninase and is in the second transgene position, with no transgenes in the first or third transgene positions. Methionine depletion by this virus is believed to selectively lead to apoptosis in cancer cells, expanding the anti-tumor effect to the entire tumor microenvironment.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgene encodes IL-12 and is in the first transgene position, with no transgenes in the second or third transgene positions. This virus is believed to induce T cell activation and proliferation, as well as differentiation of T cells into Th1 cells.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-12 in the first transgene position and adenosine deaminase in the second position, with no transgene in the third transgene position. This virus is believed to induce T cell activation and proliferation, as well as differentiation of T cells into Th1 cells. This virus is also believed to prevent immune suppression and prolong T cell engagement at the tumor site, enhancing anti-tumor immunity.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-12 in the first transgene position and methioninase in the second position, with no transgene in the third transgene position. This virus is believed to induce T cell activation and proliferation, as well as differentiation of T cells into Th1 cells. Methionine depletion by this virus is believed to selectively lead to apoptosis in cancer cells, expanding the anti-tumor effect to the entire tumor microenvironment.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-12 in the first transgene position and IL-15 in the second position, with no transgene in the third transgene position. This virus is believed to induce T cell activation and proliferation, as well as differentiation of T cells into Th1 cells. This virus is also believed to induce proliferation, activation and survival of Cd8+ T, NK, NKT, and dendritic cells; promote the formation of memory T cells; and maintain the vitality of amplification of cytotoxic T lymphocytes.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-12 in the first transgene position and GM-CSF in the second position, with no transgene in the third transgene position. This virus is believed to induce T cell activation and proliferation, as well as differentiation of T cells into Th1 cells. This virus is also believed to improve antigen presentation through recruitment and activation of dendritic cells and macrophages.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position and CCL5/RANTES in the second position, with no transgene in the third transgene position. This virus is believed to induce proliferation, activation and survival of Cd8+ T, NK, NKT, and dendritic cells; promote the formation of memory T cells; and maintain the vitality of amplification of cytotoxic T lymphocytes. This virus is also believed to promote recruitment and infiltration of leukocytes into tumors.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position, CCL5/RANTES in the second position, and adenosine deaminase in the third transgene position. This virus is believed to induce proliferation, activation and survival of Cd8+ T, NK, NKT, and dendritic cells; promote the formation of memory T cells; and maintain the vitality of amplification of cytotoxic T lymphocytes. This virus is also believed to promote recruitment and infiltration of leukocytes into tumors. This virus is also believed to prevent immune suppression and prolong T cell engagement at the tumor site, enhancing anti-tumor immunity.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position, CCL5/RANTES in the second position, and methioninase in the third transgene position. This virus is believed to induce proliferation, activation and survival of Cd8+ T, NK, NKT, and dendritic cells; promote the formation of memory T cells; and maintain the vitality of amplification of cytotoxic T lymphocytes. This virus is also believed to promote recruitment and infiltration of leukocytes into tumors. Methionine depletion by this virus is also believed to selectively lead to apoptosis in cancer cells, expanding the anti-tumor effect to the entire tumor microenvironment.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position, adenosine deaminase in the second position, and methioninase in the third transgene position. This virus is believed to induce proliferation, activation and survival of Cd8+ T, NK, NKT, and dendritic cells; promote the formation of memory T cells; and maintain the vitality of amplification of cytotoxic T lymphocytes. This virus is also believed to prevent immune suppression and prolong T cell engagement at the tumor site, enhancing anti-tumor immunity. Methionine depletion by this virus is also believed to selectively lead to apoptosis in cancer cells, expanding the anti-tumor effect to the entire tumor microenvironment.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode GM-CSF in the first transgene position, adenosine deaminase in the second position, and methioninase in the third transgene position. This virus is believed to improve antigen presentation through recruitment and activation of dendritic cells and macrophages. This virus is also believed to prevent immune suppression and prolong T cell engagement at the tumor site, enhancing anti-tumor immunity. Methionine depletion by this virus is also believed to selectively lead to apoptosis in cancer cells, expanding the anti-tumor effect to the entire tumor microenvironment.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position, GM-CSF in the second position, and adenosine deaminase in the third transgene position. This virus is believed to induce proliferation, activation and survival of Cd8+ T, NK, NKT, dendritic cells, promote memory T cells, and maintain the vitality of amplification cytotoxic T lymphocytes. This virus is also believed to improve antigen presentation through recruitment and activation of dendritic cells and macrophages. This virus is also believed to prevent immune suppression and prolong T cell engagement at the tumor site, enhancing anti-tumor immunity.

In an embodiment, the virus is an oncolytic virus where the virus is an Ad5 virus with genomic deletions to confer replication selectivity, i.e., a 24-bp deletion in the E1A gene prevents interference with the p53 pathway, whereas a deletion in the E3 gene enhances the immune response. The transgenes encode IL-15 in the first transgene position, GM-CSF in the second position, and methioninase in the third transgene position. This virus is believed to induce proliferation, activation and survival of Cd8+ T, NK, NKT, dendritic cells, promote memory T cells, and maintain the vitality of amplification cytotoxic T lymphocytes. This virus is also believed to improve antigen presentation through recruitment and activation of dendritic cells and macrophages. Methionine depletion by this virus is also believed to selectively lead to apoptosis in cancer cells, expanding the anti-tumor effect to the entire tumor microenvironment.

As used herein, the phrase “essential-agent-depletion-enzyme” refers to an enzyme that is capable of depleting, removing or otherwise negating the effect of an essential agent for physiologic functioning, such as the agents: methionine, asparagine, adenosine, uric acid, and the like. In particular embodiments, the essential-agent-depletion-enzyme is selected from one or more of methioninase, asparaginase, adenosine-deaminase, cytosine deaminase and/or uricase; and the like. For example, in one embodiment, the essential-agent-depletion-enzyme is methionase; and further comprise an essential-agent-depletion-enzyme selected from methioninase, asparaginase, adenosine-deaminase, and cytosine deaminase.

In particular embodiments, the replication-competent oncolytic virus further comprises a coding-region for a prodrug-converting-enzyme that converts a prodrug into an active chemotherapeutic agent. As used herein the term “prodrug” refers to a drug substance that needs to be converted into the pharmacologically active agent by metabolic or physicochemical transformation. As used herein, the phrase “prodrug-converting-enzyme” refers to an enzyme that converts a pro-drug. The invention gene-directed enzyme-prodrug therapy is comprised of three components; the prodrug to be activated, the enzyme used for activation, and the delivery system for the corresponding gene.

In certain embodiments of the invention replication-competent oncolytic virus, the prodrug-converting-enzyme and prodrug combination are selected from the group consisting of: herpes simplex virus type 1 thymidine kinase/ganciclovir; (ii) cytosine deaminase/5-fluorocytosine; (iii) cytochrome P450/cyclophosphamide or ifosfamide; (iv) guanine phosphoribosyl-transferase/6-thioxantine; (v) bacterial nitroreductase (NTR) with 5-(azaridin-1-yl)-2,4-dinitrobenzamide (CB1954); (vi) carboxylesterase/CPT-11; (vii) Escherichia coli purine nucleoside phosphorylase/purine analogs. In particular embodiments, the virus is selected from VSV, Adenovirus, HSV, Vaccinia virus, or the like. In another embodiment, the prodrug-converting-enzyme and prodrug combination are cytosine deaminase/5-fluorocytosine or fluorocytodine. In particular embodiments of the replication-competent oncolytic virus, the cancer-selective deletion is selected from Delta51; E1B-55 kDa gene deletion (e.g., as set forth Mulvihill et al., Gene Therapy, 8:308-315 (2001); CR1 (Delta-39); CR2 (Delta-24); Delta-24/39 (as set forth in Jiang et al., Neoplasia, Vol. 7, Issue 8, August 2005, pgs 723-729.).

Using the invention method, the systemically administered prodrug is ideally converted to the active chemotherapeutic agent only in cancer cells, thereby allowing a maximal therapeutic effect while limiting systemic toxicity. Exemplary prodrug converting enzymes/prodrug combinations for use herein include: (i) herpes simplex virus type 1 thymidine kinase/ganciclovir; (ii) cytosine deaminase/5-fluorocytosine; (iii) cytochrome P450/cyclophosphamide or ifosfamide; (iv) guanine phosphoribosyl-transferase/6-thioxantine; (v) bacterial nitroreductase (NTR) with 5-(azaridin-1-yl)-2,4-dinitrobenzamide (CB1954); (vi) carboxylesterase/CPT-11; (vii) Escherichia coli purine nucleoside phosphorylase/purine analogs.

For example, in one embodiment, an exemplary gene therapy approach for use in treating brain tumors is gene-directed enzyme-prodrug therapy also known as suicide gene therapy. This approach is comprised of three components; the prodrug to be activated, the enzyme used for activation, and the delivery system for the corresponding gene (Anderson, 2000). Using this embodiment, the systemically administered prodrug is ideally converted to the active chemotherapeutic agent only in cancer cells, thereby allowing a maximal therapeutic effect while limiting systemic toxicity. Several suicide gene therapy embodiments are contemplated for use herein: (i) herpes simplex virus type 1 thymidine kinase/ganciclovir; (ii) cytosine deaminase/5-fluorocytosine; (iii) cytochrome P450/cyclophosphamide or ifosfamide; (iv) guanine phosphoribosyl-transferase/6-thioxantine; (v) nitroreductase/CB1954; (vi) carboxylesterase/CPT-11; (vii) Escherichia coli purine nucleoside phosphorylase/purine analogs.

In some embodiments, the recombinant virus genome or nucleic acid encodes at least one transgene. In some embodiments the at least one transgene encoded by the recombinant virus genome or nucleic acid can encode a cytokine, chemokine, enzyme, or any combination thereof. In some embodiments, the recombinant virus genome or nucleic acid can encode one, two, three, or more than three transgenes. In some embodiments, the cytokine or chemokine is GM-CSF, IL-12, IL-15, CCL4, CCL5, CXCL11, 4-1BBL, TNF-α, TNF-γ, or any combination thereof. In some embodiments, the enzyme is methioninase, adenosine deaminase, cytosine deaminase, or any combination thereof.

Inclusion of adenosine deaminase as a transgene prevents immune suppression and prolongs T cell engagement at the tumor site, thereby enhancing anti-tumor immunity. In particular, adenosine deaminase depletes adenosine from the tumor tissue to stop the migration of T-cells of the tumor tissue. Inclusion of methioninase as a transgene provides a targeted tumor-killing effect because methionine depletion selectively leads to apoptosis in cancer cells and expands the anti-tumor effect to the entire anti-tumor microenvironment. In particular, methionine depletion by methioninase arrests cells at the G2/S phase to enhance viral replication and selectively trigger apoptosis in tumor cells not infected by the recombinant virus. Depletion of amino acid methionine has been shown to be effective in the treatment of many types of cancer. The sensitivity of cancer cells to methionine depletion can be based on deletion of the genes CDKN2A (p16INK4a) and methylthioadenosine phosphorylase (MTAP), both of which are co-located on chromosome 9p21, in cancer. Deletion of MTAP makes cells hypersensitive to depletion of methionine, which is an essential amino acid obtained only through diet. Many cancer cells, especially solid tumors, have hypersensitivity to methionine depletion. Deletion of CDKN2A is one of the most common mutations encountered in cancer and is especially seen in melanoma, pancreatic adenocarcinoma, glioblastoma, non-small cell lung cancer, bladder carcinoma, and some leukemias. Moreover, it has been discovered that methionine depletion produces cell arrest in the S and G2 phases of the cell cycle, at which many cytotoxic drugs (Bertino et al. 2011; Tan et al. 1996) such as paclitaxel are the most effective, thus, making methionine depletion ideal for combinatorial therapeutic approaches (Bertino et al. 2011; Tan et al. 1996; Ortac 2013).

In some embodiments, the first transgene in the recombinant virus genome or nucleic acid encodes GM-CSF, IL-12, IL-15, TNF-α, 4-1BBL or any combination thereof. In some embodiments, a second transgene is present that encodes CCL4, CCL5, CXCL11, 4-1BBL, GM-CSF, RANTES, TNF-□, or any combination thereof. In some embodiments, a third transgene is present that encodes methioninase, adenosine deaminase, cytosine deaminase, or any combination thereof. In some embodiments, the first, second, and/or third transgene encodes a reporter gene, such as GFP, RFP, dsRed, mCherry, or luciferase. In some embodiments, the first, second, and/or third transgene encodes an immune checkpoint-related molecule, such as anti-PD1, anti-PD1-L1, anti-CTLA-4, PD-1, PD1-L1, or CTLA-4. In some embodiments, the first, second, and/or third transgene encodes ICOS or ICOS ligand. In some embodiments, the expression of at least one of the transgenes is driven by the E1 promoter, the E3 promoter, or the CMV promoter. In some embodiments, the expression of all of the transgenes is driven by the E1 promoter, the E3 promoter, the CMV promoter, or combinations thereof. In these embodiments and the related embodiments that follow, the virus or surface-engineered delivery system is used for treatment of cancer or for other immunotherapy indications. In other embodiments, the recombinant virus or surface-engineered delivery system can be administered together with (i.e., before, concurrently, or after) any other cancer or immunotherapy drug or adjuvant known in the art, such as checkpoint inhibitors including anti-PD-L1 drugs or immunotherapies such as GM-CSF, IL-2, IL-12, IL-15, TNF-α, TNf-γ, CTLA-4, PD-1, ICOS. Such immunotherapies may be given intravenously prior to intravenous administration of the recombinant virus or surface-engineered delivery system Such combinations are set forth in FIG. 19.

In other embodiments, the recombinant virus or surface-engineered delivery system can be combined with other cancer therapies, radiation therapy, hormone therapy, or chemotherapy. Suitable additional therapeutic agents include, but are not limited to, therapeutic agent is selected from the group consisting of chemotherapeutic agents, CDK inhibitors, anti-inflammatory agents, antibiotics, antiviral agents, immunological agents, vitamins, growth factors, and hormones. Thus, the provided methods include, optionally, administering to the subject known anticancer compounds or chemotherapeutic agents. Chemotherapeutic agents, include, but are not limited to 5-fluorouracil; mitomycin C; methotrexate; hydroxyurea; cyclophosphamide; dacarbazine; mitoxantrone; anthracyclins (epirubicin and doxurubicin); antibodies to receptors, such as herceptin; etoposide; pregnasome; hormone therapies such as tamoxifen and anti-estrogens; interferons; aromatase inhibitors; progestational agents; and LHRH analogs. CDK (Cyclin-dependent kinase) inhibitors are agents that inhibit the function of CDKs. Suitable CDK inhibitors for use in the provided methods include, but are not limited to, AG-024322, AT7519, AZD5438, flavopiridol, indisulam, P1446A-05, PD-0332991, and P276-00 (See., e.g., Lapenna et al., Nature Reviews, 8:547-566 (2009), which is incorporated by reference herein in its entirety). The choice of agent and dosage can be determined readily by one of skill in the art based on the given disease being treated. The combined administrations contemplates coadministration, using separate formulations or a single pharmaceutical formulation, and consecutive administration in either order, wherein preferably there is a time period while both (or all) active agents simultaneously exert their biological activities. Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions.

In embodiments, the compositions and methods of the present invention may be used in the context of hyperproliferative or neoplastic diseases/conditions including cancer and atherosclerosis. It may be desirable to combine these compositions with other agents effective in the treatment of those diseases and conditions. For example, the treatment of a cancer may be implemented with therapeutic compounds of the present invention and other anti-cancer therapies, such as anti-cancer agents or surgery.

Administration of the recombinant virus or surface-engineered delivery system of the present invention to a patient will follow general protocols for the administration of that particular secondary therapy, taking into account the toxicity, if any, of the virus treatment. It is expected that the treatment cycles would be repeated as necessary. It also is contemplated that various standard therapies, as well as surgical intervention, may be applied in combination with the described cancer or tumor cell therapy.

Anti-Cancer Therapy

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with virus or viral construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the virus and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that poxvirus therapy could be used similarly in conjunction with chemotherapeutic, radiotherapeutic, immunotherapeutic, or other biological intervention, in addition to other pro-apoptotic or cell cycle regulating agents.

Alternatively, a viral therapy may precede or follow the other treatment by intervals ranging from minutes to weeks. In embodiments where the other agent and virus are applied separately to the cell, one would generally ensure that a significant period of time did not expire between the time of each delivery, such that the agent and virus would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Chemotherapy

Cancer therapies also include a variety of combination therapies with both chemical and radiation-based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

Radiotherapy

Other factors that cause DNA damage and have been used extensively include what are commonly known as γ-rays, X-rays, proton beams, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Tumor cell lysates may also be used in an antigenic composition.

An alternative aspect of immunotherapy is to combine anticancer effects with immune stimulatory effects. Immune stimulating molecules include: cytokines such as IL-2, IL-4, IL-12, GM-CSF, IFNγ, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Representative combinations with the recombinant viruses of the present invention are shown in FIG. 19

Examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005 and 5,739,169; Hui and Hashimoto, 1998; Christodoulides et al., 1998), cytokine therapy (e.g., interferons α, β and γ; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor (Dillman, 1999). Combination therapy of cancer with herceptin and chemotherapy has been shown to be more effective than the individual therapies. Thus, it is contemplated that one or more anti-cancer therapies may be employed with the therapies related to the recombinant virus or surface-engineered delivery system described herein.

Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie and Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988).

Active Immunotherapy

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

Adoptive Immunotherapy

In adoptive immunotherapy, the patient's circulating lymphocytes, or tumor infiltrated lymphocytes, are isolated in vitro, activated by lymphokines such as IL 2 or transduced with genes for tumor necrosis, and readministered (Rosenberg et al., 1988; 1989). To achieve this, one would administer to an animal, or human patient, an immunologically effective amount of activated lymphocytes in combination with an adjuvant incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

Genes

In yet another embodiment, the secondary treatment to be combined with the recombinant virus or surface-engineered delivery system is a gene therapy in which a therapeutic polynucleotide is administered before, after, or at the same time as the recombinant virus or surface-engineered delivery system is administered. Delivery of the recombinant virus or surface-engineered delivery system of the present invention in conjunction with a vector encoding one of the following gene products will have a combined anti-cancer effect on target tissues. Alternatively, the recombinant virus or surface-engineered delivery system may be engineered as to include a nucleic acid sequence expressing the therapeutic polynucleotide. A variety of proteins are encompassed within the invention, some of which are described below.

Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, however. In one embodiment of the present invention, it is contemplated that anti-sense mRNA directed to a particular inducer of cellular proliferation is used to prevent expression of the inducer of cellular proliferation.

Inhibitors of Cellular Proliferation

The tumor suppressor oncogenes function to inhibit excessive cellular proliferation. The inactivation of these genes destroys their inhibitory activity, resulting in unregulated proliferation. Tumor suppressors include p53, p16 and C-CAM. Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bel 2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl 2 acts to suppress cell death triggered by a variety of stimuli. Also, it now is apparent that there is a family of Bcl-2 cell death regulatory proteins which share in common structural and sequence homologies. These different family members have been shown to either possess similar functions to Bel 2 (e.g., BclXL, BclW, BelS, Mel-1, Al, Bfl-1) or counteract Bcl 2 function and promote cell death (e.g., Bax, Bak, Bik, Bim, Bid, Bad, Harakiri).

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, pre-cancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing ability of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors arc focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as viral therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment.

In other embodiments, rather than all of the cytokines, chemokines, and/or enzymes being encoded in the same recombinant virus genome or nucleic acid, or delivered within the same surface-engineered delivery system, different transgenes each encoding a different cytokine, chemokine, and/or enzyme may be encoded in different recombinant virus genomes or nucleic acids, or carried in different surface-engineered delivery systems, which can then be co-administered to provide a combined effect. In other embodiments, one or more of the cytokines, chemokines, or enzymes maybe be delivered as a protein or peptide, for example in a pharmaceutical or dietary formulation.

Accordingly, also provided herein are invention recombinant viruses, wherein the recombinant viruses comprise combinations of at least 1, at least 2 transgenes set forth in FIG. 19; or that can be combined in any of the genomes of an virus set forth herein, e.g., in Ad5, herpes virus, enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, and hepatitis A virus. In other embodiments, the surface-engineered recombinant virus is an oncolytic virus and is selected from the group consisting of: NG-641 (PsiOxus), and Imlygic (talimogene laherparepvec), and the like.

Also provided herein is a method of engineering a tumor microenvironment of a patient in need thereof, said method comprising administering a combination of a first and second recombinant viruses to the patient,
wherein the first recombinant virus is an oncolytic virus comprising a coding-region for an essential-agent-depletion-enzyme and a cancer-selective deletion; and
wherein the second recombinant virus comprises at least one of:
a coding-region for a prodrug-converting-enzyme that converts a prodrug into an active chemotherapeutic agent; or
a coding-region for an essential-agent-depletion-enzyme.

As used herein, the phrase “Ad5 virus” refers to the adenovirus serotype 5 that have been repeatedly used in humans to induce robust T cell-mediated immune (CMI) responses, all while maintaining an extensive safety profile (see, e.g., U.S. Pat. No. 9,605,276, and the like). Ad5 vectors can be reliably manufactured in large quantities and are stable for storage and delivery for outpatient administration.

In certain embodiments, the adenovirus vectors contemplated for use in the present invention include E1 and E3 deleted adenovirus vectors that have a deletion in the E1 and E3 region of the Ad genome and, optionally, the E2b region. In particular embodiments used herein, these deletions are referred to herein as a “cancer-selective deletion.” In some cases, such vectors do not have any other regions of the Ad genome deleted. In another embodiment, the adenovirus vectors contemplated for use in the present invention include E1 deleted adenovirus vectors that have a deletion in the E1 region of the Ad genome and, optionally, deletions in the E3, E4 and/or E2b regions. In some cases, such vectors have no other regions deleted. In another embodiment, the adenovirus vectors contemplated for use in the present invention include E3 deleted adenovirus vectors that have a deletion in the E3 region of the Ad genome and, optionally, deletions in the E1, E4 and/or E2b regions. In some cases, such vectors have no other regions deleted. In a further embodiment, the adenovirus vectors contemplated for use in the present invention include adenovirus vectors that have a deletion in the E1 and E3 regions of the Ad genome and, optionally, deletions in the E2b and/or partial or complete removal of the E4 regions. In some cases, such vectors have no other deletions. In an additional embodiment, the adenovirus vectors contemplated for use in the present invention include adenovirus vectors that have a deletion in the Eta, E2b and/or E4 regions of the Ad genome. In some cases, such vectors have no other deletions. In one embodiment, the adenovirus vectors for use herein comprise vectors having the E1 and/or DNA polymerase functions of the E2b region deleted. In some cases, such vectors have no other deletions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase and/or the preterminal protein functions deleted. In some cases, such vectors have no other deletions. In one particular embodiment, the adenovirus vectors contemplated for use herein have at least a portion of the E2b region and/or the E1 region deleted. In some cases, such vectors are not “gutted” adenovirus vectors. In this regard, the vectors may have both the DNA polymerase and the preterminal protein functions of the E2b region deleted. In an additional embodiment, the adenovirus vectors for use in the present invention include adenovirus vectors that have a deletion in the E1, E2b and/or 100K regions of the adenovirus genome. In one embodiment, the adenovirus vectors for use herein comprise vectors having the E1, E2b and/or protease functions deleted. In some cases, such vectors have no other deletions. In a further embodiment, the adenovirus vectors for use herein have the E1 and/or the E2b regions deleted, while the fiber genes have been modified by mutation or other alterations (for example to alter Ad tropism). Removal of genes from the E3 or E4 regions may be added to any of the mentioned adenovirus vectors. In certain embodiments, the adenovirus vector may be a “gutted” adenovirus vector.

In certain embodiments, the virus can express additional cytokines (e.g., at least one subunit of IL12, GM-CSF and the like) or/and chemokines together with the at least one subunit of Spike protein to enhance improve the tumor engagement.

As used herein, the phrase “E1 deleted” or “a functional portion of the E1 gene is deleted,” or grammatical variations thereof, refers to a specific DNA sequence that is mutated in such a way so as to prevent expression and/or function of at least one E1 gene product. Thus, in certain embodiments, “E1 deleted” is used in relation to a specific DNA sequence that is deleted (removed) from the Ad genome. E1 deleted or “containing a deletion within the E1 region” refers to a deletion of at least one base pair within the E1 region of the Ad genome. Thus, in certain embodiments, more than one base pair is deleted and in further embodiments, at least 20, 30, 40, 60, 70, 80, 90, 100, 110, 120, 130, 140, or 150 base pairs are deleted. In another embodiment, the deletion is of more than 150, 160, 170, 180, 190, 200, 250, or 300 base pairs within the E1 region of the Ad genome. An E1 deletion may be a deletion that prevents expression and/or function of at least one E1 gene product and therefore, encompasses deletions within exons of encoding portions of E1-specific proteins as well as deletions within promoter and leader sequences. In certain embodiments, an E1 deletion is a deletion that prevents expression and/or function of one or both of a trans-acting transcriptional regulatory factor of the E1 region. In a further embodiment, “E1 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein. In particular embodiments used herein, these E1 deletions are referred to herein as a “cancer-selective deletion.”

As used herein, the phrase “E3 deleted” or “a functional portion of the E3 gene is deleted,” or grammatical variations thereof, refers to a specific DNA sequence that is mutated in such a way so as to prevent expression and/or function of at least one E3 gene product. Thus, in certain embodiments, “E3 deleted” is used in relation to a specific DNA sequence that is deleted (removed) from the Ad genome. E3 deleted or “containing a deletion within the E3 region” refers to a deletion of at least one base pair within the E3 region of the Ad genome. Thus, in certain embodiments, more than one base pair is deleted and in further embodiments, at least 20, 30, 40, 60, 70, 80, 90, 100, 110, 120, 130, 140, or 150 base pairs are deleted. In another embodiment, the deletion is of more than 150, 160, 170, 180, 190, 200, 250, or 300 base pairs within the E3 region of the Ad genome. An E3 deletion may be a deletion that prevents expression and/or function of at least one E3 gene product within the expression cassette and therefore, encompasses deletions within exons of encoding portions of E3-specific proteins as well as deletions within promoter and leader sequences. In certain embodiments, an E3 deletion is a deletion that prevents expression and/or function of at least one of the host immune response modulating proteins of the E3 region (see, e.g., Arnberg, PNAS, 2013, 110(50):19976-19977). In a further embodiment, “E3 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As used herein, the phrase “E2b deleted” or “a functional portion of the E2b gene is deleted,” or grammatical variations thereof, refers to a specific DNA sequence that is mutated in such a way so as to prevent expression and/or function of at least one E2b gene product. Thus, in certain embodiments, “E2b deleted” is used in relation to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus, to be “deleted” in a particular region of the Ad genome, as used herein, refers to a specific DNA sequence that is mutated in such a way so as to prevent expression and/or function of at least one gene product encoded by that region. In certain embodiments, to be “deleted” or containing a “viral-genome-deletion” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or containing a “viral-genome-deletion” within a particular region refers to a deletion of at least one base pair within that region of the genome of the virus, e.g., the Ad genome, and the like. Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus, deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein. Deletions or mutations in the Ad genome can be within one or more of E1a, E1b, E2a, E2b, E3, E4, L1, L2, L3, L4, L5, TP, POL, IV, and VA regions.

The deleted adenovirus vectors of the present invention can be generated using recombinant techniques known in the art (see e.g., Amalfitano et al., 1998 J. Virol. 72:926-933; Hodges, et al., 2000 J Gene Med 2:250-259).

Also provided herein is a surface-engineered delivery system, said system comprising:

a payload; and an artificial coating layer surrounding the payload, wherein the artificial coating is selected from the group consisting of: silica, titanium oxide and calcium phosphate.

In an embodiment, the payload is a recombinant virus having a recombinant genome or the recombinant genome itself, and the artificial coating layer surrounds the recombinant virus and/or recombinant genome.

In another embodiment, the payload is at least one nucleic acid selected from mRNA, siRNA, miRNA, small nuclear RNA, plasmid DNA, antisense oligodeoxynucleotides, or any other nucleic acid known to those skilled in the art. In a further embodiment, the nucleic acid is complexed with a cation to form a polyion complex. In certain embodiments, the cation is a cationic polymer such as polyethylenimine or any cationic polymer known to those of skill in the art to be able to generate cationic polyion complexes with nucleic acids. In certain embodiments, the cation is a cationic lipid, such as any cationic lipid known to those of skill in the art to be able to generate cationic complexes with nucleic acids.

As used herein, the phrase “surface-engineered delivery system” refers to any delivery system where an artificial coating layer encapsulates a naturally occurring or recombinant virus, or a nucleic acid. The invention surface-engineered delivery systems provided herein can function as one or more of a vaccine, immunotherapy, oncolytic virus, nucleic acid medicine, or viral or nonviral gene therapy.

As used herein, the phrase “surface-engineered recombinant virus” refers to any recombinant virus that contains an artificial coating layer encapsulating either the entire native-virus or naturally occurring virus, whether enveloped or non-enveloped, or encapsulating a previously-enveloped-capsid isolated from a native-virus. The invention surface-engineered recombinant viruses provided herein can function as one or more of a vaccine, oncolytic virus and/or a viral vector or vehicle for gene therapy.

As used herein, a “native-virus” or “naturally occurring virus” refers to any virus, wild-type or recombinant, that is produced in a cell and either buds from a cell or can be isolated once a cell lyses.

As used herein, “natively non-enveloped virus” refers to any virus, wild-type or recombinant, that does not naturally contain an envelope and is produced in a cell and either buds from a cell or can be isolated once a cell lyses.

As used herein, the phrase “recombinant virus” or “viral vector” refers to any virus, whether enveloped or non-enveloped, that has had its genome manipulated or engineered, such as by the insertion or deletion of particular nucleic acid regions resulting in a “recombinant genome.” For example, deletion of viral genes, such as a “cancer-selective deletion” as used herein, that are essential for viral replication results in replication-defective recombinant viruses. Recombinant viruses can also contain nucleic acid insertions coding for functional therapeutic proteins to replace the corresponding defective proteins, or coding for selectable markers, or the like.

Suitable recombinant viruses (viral vectors) for use herein for surface-engineering, include all recombinant viruses, whether replication-competent, replication-defective, enveloped or non-enveloped, such as those that are currently in clinical trials or commercially available, such as: Enadenotucirev oncolytic virus (i.e., NG-641; PsiOxux; see US 20190233536 A1, which is incorporated herein by reference in its entirety for all purposes), Ad26 (Janssen/J&J), Imlygic (Amgen), and the like. In particular embodiments, the surface-engineered recombinant virus is selected from the group consisting of: enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus.

In other embodiments, the surface-engineered recombinant virus is an oncolytic virus and is selected from the group consisting of: NG-641 (PsiOxus), Imlygic (talimogene laherparepvec), Pexa-Vec (Transgene/Sillajen), Reolysin (Oncolytics Biotech), DS-1647 (Daiichi Sankyo), TG1042 (Transgene/Ascend), Cavatak (Merck), GL-ONC1 (Genelux), Marabex (Turnstone/Abbvie), ORCA-010 (Orca/VCN), ParvOryx (Oryx), LOAd703 (Lokon Pharma), PV701 (Wellstat Group), MV-NIS (Vyriad), ONCOS-102 (Targovax), Seprehvir (Sorrento), Enadenotucirev (Psioxus), CG0070 (Cold Genesys), Telomelysin (Oncolys Biopharma), JX-929 (Sillaj en), VSV Cancer Project (AstraZeneca), Ad-VirRx 007 (Multivir), NG-348 (Psioxus/BMS), VSV-GP (Viratherapeutics), RP1/RP2/RP3 (Replimune) and WO-12 (Western Oncolytics/Pfizer).

In particular embodiments, the surface-engineered recombinant virus is a Maraba virus or other rhabdovirus, which can be Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus, or combinations thereof. In certain aspects, rhabdovirus can refer to the supergroup of Dimarhabdovirus (defined as rhabdovirus capable of infection both insect and mammalian cells). In particular aspects the rhabdovirus is a Caraj as virus, Maraba virus, Muir Springs virus, and/or Bahia grande virus, including variants thereof. Several newly identified rhabdoviruses are much more efficient at killing particular cancers or cancer cell lines than VSV. Also, VSV and attenuated mutants of VSV are neurovirulent and cause CNS pathology in rodents and primates. Several rhabdoviruses do not infect the CNS (i.e., Muir Springs and Bahia Grande: Kerschner et al., 1986), and demonstrate a more acceptable safety profile. In addition, therapies based on rhabdoviruses can be used to treat cancers of the CNS, both primary and secondary. Such rhabdoviruses, and the surface-engineered versions described herein, can also be co-administered with a histone deacetylase inhibitor to enhance the immune response. Such co-administration is described in U.S. Pat. No. 9,821,054.

Such Maraba virus can be an oncolytic Maraba virus encoding a variant M and/or G protein having an amino acid identity of at least or at most 20, 30, 40, 50, 60, 65, 70, 75, 80, 85, 90, 92, 94, 96, 98, 99, 100%, including all ranges and percentages there between, to the M or G protein of Maraba virus. In certain aspects amino acid 242 of the Maraba G protein is mutated. In further aspects amino acid 123 of the M protein is mutated. In still further aspects both amino acid 242 of the G protein and amino acid 123 of the M protein are mutated. Amino acid 242 can be substituted with an arginine (Q242R) or other amino acid that attenuates the virus. Amino acid 123 can be substituted with a tryptophan (L123W) or other amino acid that attenuates the virus. In certain aspects two separate mutations individually attenuate the virus in normal healthy cells. Upon combination of the mutants the virus becomes more virulent in tumor cells than the wild type virus. Such Maraba viruses are described in U.S. Pat. Nos. 9,896,664 and 9,045,729. In other embodiments, the rhabdovirus can be an oncolytic recombinant rhabdovirus encoding the Isfahan virus G protein, the Maraba virus G protein, or Muir Springs virus G protein, where the rhabdovirus further encodes M, P, N and L proteins from vesicular stomatitis virus (VSV). Such rhabdoviruses are described in U.S. Pat. Nos. 9,572,883, 9,572,883, and 8,481,023.

In other embodiments, such Maraba virus or rhabovirus can comprise a nucleic acid that is capable of expressing a MAGEA3 protein that includes at least one MAGEA3 tumor-associated epitope, for examples FLWGPRALV, KVAELVHFL, EGDCAPEEK, KKLLTQHFVQENYLEY, and/or RKVAELVHFLLLKYR, where the MAGEA3 protein is capable of inducing an immune response in a patient in a heterologous prime-boost format. A second virus may be used in combination with the Maraba virus or rhabovirus, where the second virus is a Maraba MG1 virus comprising a nucleic acid that is capable of expressing a MAGEA3 protein that includes at least one MAGEA3 tumor-associated epitope, for example FLWGPRALV, KVAELVHFL, EGDCAPEEK, KKLLTQHFVQENYLEY, and/or RKVAELVHFLLLKYR. Such viruses are described in U.S. patent Ser. No. 10/363,293. MAGEA3, Human Papilloma Virus E6/E7 fusion protein, human Six-Transmembrane Epithelial Antigen of the Prostate protein, and Cancer Testis Antigen 1, are all able to be used in a heterologous prime-boost setting to induce an immune response in a mammal, and thus the Maraba virus or rhabovirus can comprise a nucleic acid that is capable of expressing a Human Papilloma Virus E6/E7 fusion protein or other HPV tumor-associated antigen, human Six-Transmembrane Epithelial Antigen of the Prostate protein, Cancer Testis Antigen 1, an oncofetal antigen, a surface glycoprotein, a cell surface marker, a melanoma-associated antigen, cancer-testes antigen, an oncogene, a viral oncogene, dopachrome tautomerase (DCT), GP100, MART 1, alphafetoprotein (AFP) and/or carcinoembryonic antigen (CEA), all containing the respective immune epitopes, in addition to or instead of the MAGEA3 protein described above. The second virus can also be a Vesiculovirus comprising a nucleic acid capable of expressing an HPV tumor-associated antigen. Such viruses are described in U.S. patent Ser. No. 10/646,557, 10,660,947, 9,707,285, and 10,925,946. In another embodiment using a two-virus combination, the first virus can be an oncolytic vaccinia virus (VV) expressing a functional vaccinia virus type 1 interferon (IFN) binding protein, and the second virus can be an IFN-sensitive oncolytic vesicular stomatitis virus (VSV) comprising a mutation in the gene encoding matrix (M) protein rendering the encoded polypeptide unable to block IFN gene expression. In further embodiments, the oncolytic VSV has an attenuating deletion of methionine 51 of the matrix protein (VSVdeltaM51). In still further embodiments, the VV does not express functional thymidine kinase and functional vaccinia growth factor. This combination of viruses induces a contemporaneous synergistic lytic infection in proliferating cells such as cancer cells. Such viruses are described in U.S. patent Ser. No. 10/603,351.

In other embodiments, the surface engineered virus can be an oncolytic virus whose genome comprises an open reading frame that encodes an FGF2 protein or a functional variant thereof, where the FGF2 protein or functional variant thereof further comprises an immunoglobulin signal peptide. The genome of such a virus can further comprise an open reading frame that encodes a Type 1 interferon scavenger such as a B18R protein. Such a virus can be a rhabdovirus, a vaccinia virus, or herpes simplex virus-1. Such a virus can be The virus may be a rhabdovirus, a vaccinia virus, herpes simplex virus-1, reovirus, measles virus, Modified Vaccinia Ankara virus, NewCastle Disease virus, influenza virus, West Nile virus, dengue virus, HIV, rabies virus, hepatitis virus, or poliovirus. The rhabdovirus may be vesicular stomatitis virus, VSVΔ51, VSV IFN-β, maraba virus, or MG1 virus. Such viruses show enhanced virus replication in permissive cells that express a receptor to fibroblast growth factor 2 (FGF2) protein. The permissive cell may express FGF Receptor 1, FGF Receptor 3, or both. The permissive cells that express a receptor to FGF2 protein may be cancer cells, such as adenocarcinoma cells, pancreatic carcinoma cells, ovarian carcinoma cells, renal carcinoma cells, or colon carcinoma cells. The permissive cells that express a receptor to FGF2 protein may be activated fibroblast cells, such as activated human fetal fibroblast cells or cancer-associated fibroblast cells. The activated human fetal fibroblast cells may be WI38 cells or MRCS cells. Such viruses are described in U.S. patent Ser. No. 10/604,741 and 10,066,214.

Accordingly, provided herein is a surface-re-engineered recombinant rhabdovirus comprising:
a previously-enveloped-capsid from a recombinant oncolytic rhabdovirus encoding an M or G protein; and
an artificial coating layer surrounding the previously-enveloped-capsid. In one embodiment, the surface-engineered recombinant rhabdovirus virus comprises an artificial coating selected from the group consisting of: silica, titanium oxide and calcium phosphate. In another embodiment, the invention rhabdovirus further comprises up to three transgenes, as described here, encoding any combination of proteins selected from methioninase, adenosine deaminase, cytosine deaminase, GM-CSF, IL-12, IL-15, TNF-α, 4-1BBL, CCL4, CCL5/RANTES, and CXCL11.

Also provided herein are methods of treating cancer in a patient in need thereof, comprising administering to the patient an invention surface-re-engineered recombinant rhabdovirus set forth hereinabove.

In particular embodiments, the surface-engineered recombinant virus is an adenovirus that is E1B-55k-impaired and/or E4-ORF3-impaired but not replication-impaired in a p53-impaired cell, i.e., the adenovirus selectively replicates in a p53-impaired cell. For E4-ORF3 impairment, such an adenovirus may express a mutated E4-ORF3 gene product or have an E4-ORF3 gene that is wholly or partially deleted. For E1B-55k impairment, such an adenovirus may express a mutated E1B-55k gene product or have an E1B-55k gene that is wholly or partially deleted. Such an adenovirus can be used to treat a cancer with reduced expression or activity of p53, including lung cancer, skin cancer, or malignant or pre-malignant breast cancer. Such adenoviruses are described in U.S. Pat. No. 9,187,733. Provided herein is a novel, genetically encoded, inducible chemical adapter system that targets infection to multiple cellular receptors. Oncolytic viral therapy has the potential to destroy a tumor mass of unlimited size, but only if the virus crosses the vasculature and infection spreads from one cancer cell to another. Thus, the reliance of previous adenoviral vectors on a single cellular receptor for their uptake limits their therapeutic potential. Altering the chemistry and binding of viral capsids so that infection can be specifically targeted to any cell type is a major breakthrough. This is achieved by using a known property of the cancer drug rapamycin to dimerize heterologous proteins with FKBP and FRB domains (e.g., viruses that express a Fiber-FRB capsid protein fusion together with re-targeting ligands fused to-FKBP). These viruses infect cells via multiple re-targeting ligands upon rapamycin treatment, which is a rational and powerful combination of chemical and viral weapons directed to novel cancer therapy. Moreover, these technologies enable multi-protein complexes and entire pathways to be assembled, delivered and co-expressed in any cell type via adenoviral infection. These technologies allow ‘next generation’ oncolytc viruses to be developed that specifically kill p53 mutant tumors and pre-malignant breast cancer cells, and which have the potential to save the lives of many cancer patients. In another embodiment, the adenovirus can be further modified to include any one or more of the transgenes set forth herein, e.g., in Table 1 or FIGS. 19-21, and the like, to additionally provide the advantages and effects described herein.

In particular embodiments, the surface-engineered recombinant virus is a a recombinant herpes simplex virus (HSV)-1 that is infected cell protein 0 (ICP0)-deficient, glycoprotein C (gC)-deficient, or both ICP0-deficient and gC-deficient. For ICP0 deficiency, the HSV-1 may have a disruption in at least one copy of the ICP0 gene that diminishes or eliminates expression of functional ICP0. In particular, such a disruption may be a complete deletion of the ICP0 gene, a partial deletion in the ICP0 gene, or an insertion or a point mutation in the ICP0 gene. The partial deletion in the ICP0 gene or the insertion or point mutation in the ICP0 gene may be in the RING finger domain coding region. For gC deficiency, the recombinant HSV-1 may have a gC gene with a deletion of the cytosine at position 186 relative to the gC gene of wild-type HSV-1, and/or it may have a gC gene with premature termination at the 175th codon relative to the gC gene of wild-type HSV-1. The recombinant HSV-1 may contain a heterologous gene encoding an immunostimulatory molecule. The recombinant HSV-1 may have a disruption in at least one copy of the ICP34.5 gene, the ICP6 gene, and/or the ICP47 gene that diminishes or eliminates expression of functional ICP34.5, ICP6 and/or ICP47. The disruption in the ICP34.5 gene, the ICP6 gene, or the ICP47 gene may be a complete deletion of the ICP34.5 gene, the ICP6 gene, or the ICP47 gene. The disruption in the ICP34.5 gene, the ICP6 gene, or the ICP47 gene may be a partial deletion in the ICP34.5 gene, the ICP6 gene, or the ICP47 gene. The disruption in the ICP34.5 gene, the ICP6 gene, or the ICP47 gene may be an insertion or a point mutation in the ICP34.5 gene, the ICP6 gene, or the ICP47 gene. In some embodiments, the recombinant HSV-1 comprises a complete deletion of the ICP0 gene and comprises a heterologous gene encoding GM-CSF. In some embodiments, the recombinant HSV-1 comprises a complete deletion of the ICP0 gene and a partial deletion of the ICP47 gene, and further comprises a heterologous gene encoding GM-CSF. The recombinant HSV-1 may be used to treat an alternative lengthening of telomeres (ALT)-dependent cancer, including a soft tissue sarcoma, a cancer of the central nervous system, or an osteosarcoma. Such HSV-1 viruses are described in U.S. patent Ser. No. 10/821,141. In another embodiment, the recombinant HSV-1 can be further modified to include any one or more of the transgenes set forth herein, e.g., in Table 1 or FIGS. 19-21, and the like, to additionally provide the advantages and effects described herein.

In some embodiments, the surface-engineered recombinant virus is a recombinant adenovirus having a modified genome having combined E1A and E4-ORF6/7 mutations together with other mutations that confer potency, tumor selectivity, and/or tumor tropism. Combining E1A and E4-ORF6/7 mutations in an Ad OV yields an improved selectivity/efficacy profile over first-generation OVs engineered with E1A-RB mutations alone by conferring E2F-dependent tumor-selective replication without negatively impacting lytic potency in cancer cells. The Rb/p16/E2F pathway is inactivated by mutations or through other mechanisms, e.g., viral mechanisms, in almost every form of human cancer. By way of example, the pathway can be inactivated through mutations in Rb, p107 mutations, p130 mutations, p16 mutations/epigenetic silencing, cyclin mutations and amplifications, CDK mutations and amplifications, mutations that downregulate cyclin dependent kinase inhibitors, mutations that upregulate E2F transcription factors and growth factor receptor pathway mutations (EGFR, RTKs, RAS, PI-3K, PTEN, RAF, MYC). However, most current chemotherapies are proliferative poisons that inhibit E2F transcriptional targets, but are also toxic to normal cells and have often devastating iatrogenic complications. Tumor mutations and small DNA virus' proteins converge in inactivating Rb. Studies with adenovirus E1A provided seminal insights into Rb and E2F. The original concept for an oncolytic adenovirus was an E1AΔLXCXE mutant but the agent is not selective, at least in primary cell cultures. E1A binds and inactivates Rb via a conserved (CR2) LXCXE motif (Whyte, et al., Nature 334(6178):124-9 (1988)), which activates E2F dependent transcription (Kovesdi et al., PNAS 84(8):2180-4 (1987)). This is thought to be the mechanism through which E1A activates E2F, diving expression of cellular and viral genes required for cellular and viral genome replication. Therefore, it was proposed that an adenovirus E1AΔCR2 mutant would selectively replicate in tumor cells that had mutations in the Rb/p16 tumor suppressor pathway (Heise et al., Nat. Med. 6(10):1134-9 (2000)). However, surprisingly, an E1AΔCR2 viral mutant still activates E2F and replicates in primary human epithelial cells (Johnson et al., Cancer Cell 1(4):325-337 (2002)). It was then found that adenoviruses encode an additional viral protein, E4orf6/7, that activates E2F independently of E1A. Previous studies had shown that E4orf6/7 binds to E2F and DP1 to activate the transcription of viral E2 promoters (Helin and Harlow, J. Virol. 68(8):5027-5035 (1994)). Because E4orf6/7 is thought to activate E2F-dependent cellular targets to drive S phase entry and viral replication, independently of E1A, adenoviruses have been designed with mutations in both E1A and E4orf6/7 to permit selective replication in tumor versus normal cells, thus providing selective anti-cancer agents. Such engineered viruses are believed to be self-perpetuating, to kill tumor cells through regulated cell death, and to produce progeny that can spread not only within the tumor but also to metastatic sites. In another embodiment, such viruses can be further modified to include any one or more of the transgenes set forth herein, e.g., in Table 1 or FIGS. 19-21, and the like, to additionally provide the advantages and effects described herein.

In such embodiments, where the surface-engineered recombinant virus is a recombinant adenovirus having a modified genome having combined E1A and E4-ORF6/7 mutations, the recombinant adenovirus can have a modified genome having (i) an adenovirus E1A promoter operably linked to a nucleic acid sequence encoding an E1A protein comprising a modification in a first Rb-binding site; and (ii) an adenovirus E4 promoter operably linked to an E4orf6/7 gene product coding sequence having a deletion or a modification of only one of the two E4orf6/7 exons, wherein the deletion of the modification impairs E4orf6/7 gene product activity and/or expression, and wherein the recombinant adenovirus selectively replicates in Rb-deficient cells. In such a virus, the E1A protein may further comprise a modification in a second Rb-binding site, where the modification is a deletion of the LXCXE motif; a deletion of amino acid residues 122-126; a deletion of amino acid residues 2-11; a substitution at residue Y47; a substitution at residue C124; or any combination of the above. In some embodiments, the modification in the E1A protein can be at least one of a substitution at residue Y47, wherein the substitution is a Y47H substitution, and a substitution at residue C124, wherein the substitution is a C124G substitution. The genome of the virus can contain a nucleic acid sequence encoding an E4orf1 protein comprising one or more modifications, where the modifications are a deletion in the C-terminal region, a deletion of the last four amino acids in the C-terminal region, and/or a deletion of residues 125-128. Such a virus includes ICVB-1042, described in U.S. patent Ser. No. 11/077,156 and/or 9187733, the first rationally engineered OV to incorporate the dual E1A and E4-ORF6/7 mutation. ICVB-1042 has been shown to infect and kill a broad range of tumor cells, including head and neck, bladder, lung and breast, suggesting that it could have potential utility in a wide range of solid tumor indications. In another embodiment, the ICVB-1040 virus can be further modified to include any one or more of the transgenes set forth herein, e.g., in Table 1 or FIGS. 19-21, and the like, to additionally provide the advantages and effects described herein.

In some embodiments, the surface-engineered virus is the the ΔE1B-55k oncolytic adenovirus ONYX-015 (Oncorine), which is used to treat p53-driven tumors. The proposed p53 tumor selectivity of Oncorine is based on E1B-55k being the critical and sole mechanism whereby p53 is inactivated in adenovirus infected cells. In another embodiment, Oncorine can be further modified to include any one or more of the transgenes set forth herein, e.g., in Table 1 or FIGS. 19-21, and the like, to additionally provide the advantages and effects described herein.

Accordingly, provided herein is a surface-engineered recombinant adenovirus, said virus comprising:
a recombinant adenovirus having a modified genome comprising:
   (i) an adenovirus E1A promoter operably linked to a nucleic acid sequence encoding an E1A protein comprising a modification in a first Rb-binding site; and
   (ii) (ii) an adenovirus E4 promoter operably linked to an E4orf6/7 gene product coding sequence having a deletion or a modification of only one of the two E4orf6/7 exons, wherein the deletion of the modification impairs E4orf6/7 gene product activity and/or expression; wherein the recombinant adenovirus selectively replicates in Rb-deficient cells; and
an artificial coating layer surrounding the recombinant adenovirus. In one embodiment, this particular surface-engineered recombinant adenovirus virus comprises an artificial coating selected from the group consisting of: silica, titanium oxide and calcium phosphate. In another embodiment, this particular adenovirus further comprises up to three transgenes, as described here, encoding any combination of proteins selected from methioninase, adenosine deaminase, cytosine deaminase, GM-CSF, IL-12, IL-15, TNF-α, 4-1BBL, CCL4, CCL5/RANTES, and CXCL11. Also provided herein are methods of treating cancer in a patient in need thereof, comprising administering to the patient an invention surface-re-engineered recombinant adenovirus set forth in this paragraph.

Surface engineering of oncolytic viruses as described herein provides benefits that help to overcome the current drawbacks of oncolytic virotherapy. Before the invention described herein, tumor site delivery by systemic administration was challenging or not possible. In the state of the art, it is also difficult to achieve the ideal immune activity while elevating antigen presentation and modifying tumor environment for efficacy. Furthermore, repeat administration for virotherapy (any virotherapy, not only oncolytic virotherapy) is challenging or not possible in the current state of the art. In the current state of the art, off-target effects and toxicity limit the applicability of many virotherapies. Application of ONCoat, e.g., an artificial coating layer, to viral therapies, as described herein, permits systemic administration. An additional advantage of such systemic administration is that metastatic cancers, and not only primary cancers, can be addressed. ONCoat also enables the use of a broader range of viruses with higher potency viruses and higher immunostimulatory effects. ONCoat allows the therapy to be designed to achieve and enhance full engagement of the immune system via direct tumor response, antigen presentation, and modulation of the microenvironment. Surface engineering of the virus using ONCoat also permits repeat administration as well as enhanced therapy with safe dosing.

Surface engineering of viruses as described herein provides both clinical and commercial benefits. Clinically, surface engineering allows viruses to be freely selected without having to consider aspects such as the virus's native tropism. Thus, viruses with a high gene carrying capability can be selected, where these viruses would not normally be able to be used. Surface engineering also allows for systemic and repeat administration by shielding the virus from the body's defenses such as pre-existing neutralizing antibodies and clearance from the bloodstream. Surface engineering results in an effective therapy with a safe dose. Surface functionalization with ligands and PEG results in tissue-specific treatment with low off-target toxicity. Commercially, surface-engineered viruses show improved stability at all typical storage or usage temperature conditions (−80° C., −20° C., 4° C., 37° C.). They also have improved stability after several freeze/thaw cycles and a longer shelf-life. They also demonstrate no deterioration over 1 year at −80° C.

One aspect of the invention provides a method of treating cancer in an individual in need thereof, comprising administering to the individual an invention replication-competent oncolytic virus, or a surface-engineered recombinant oncolytic virus; thereby treating the individual. The surface-engineered recombinant virus can any be of those described herein. The surface-engineered recombinant virus can be administered by intratumoral injection or intravenous injection. In particular embodiments, the surface-engineered recombinant virus can be administered in combination with or as an adjuvant to another anti-cancer drug. In particular embodiments of the invention oncolytic virotherapy methods of treating cancer, the same invention replication-competent oncolytic virus (see, e.g., FIG. 19 and herein after), or surface-engineered oncolytic virus is administered repeatedly at 2 or more intervals. For example, an invention replication-competent oncolytic virus (see, e.g., FIG. 19 and herein after), or an invention surface-engineered oncolytic virus can be administered repeatedly at least twice at intervals in the range of 1 week up to 3 or more years apart. In one embodiment, an invention replication-competent oncolytic virus (see, e.g., FIG. 19 and herein after), or an invention surface-engineered oncolytic virus is administered repeatedly in the range of 2 to 50 times (can be up to 100 times or more); at intervals in the range of 2 weeks up to 6 months apart. The intervals between the repeat administrations can be constant, or can vary between the respective doses of invention replication-competent oncolytic virus (see, e.g., FIG. 19 and herein after), or an invention surface-engineered oncolytic virus. Also contemplated herein, by virtue of the advantageous nature of the invention surface-engineered oncolytic viruses, is the substantially indefinite administration of the same, or substantially the same, invention surface-engineered oncolytic virus bi-annually, annually, semi-annually, every 3-months, and the like; for the remainder of the respective individual's lifespan.

In a yet further embodiment, the surface-engineered recombinant virus is a vaccine and is selected from AZD1222 (AstraZeneca), ChAdOx1-nCov19 (Oxford), Ad5-nCoV (CanSino), VSV, and Ad26 (J&J), and the like.

One aspect of the invention provides a method of vaccinating an individual or generating an immune response against one or more target antigens in an individual in need thereof, comprising administering to the individual a surface-engineered recombinant virus that is a vaccine; thereby vaccinating the individual or generating an immune response against the one or more target antigens. The surface-engineered recombinant virus can any be of those described herein. The surface-engineered recombinant virus can be administered by intramuscular, intradermal, or subdermal injection, or injection into any tissue suitable for vaccination. In one embodiment, the surface-engineered virus vaccine is administered once as a single dose. In this embodiment, there is no need for repeat administration as the level of immunity generated is durable for the lifespan of the individual.

In other embodiments of the methods of vaccination, the same surface-engineered virus vaccine is administered repeatedly at 2 or more intervals. For example, an invention surface-engineered virus vaccine can be administered repeatedly at least twice at intervals in range of 1 week up to 3 or more years apart. In one embodiment, an invention surface-engineered virus vaccine is administered repeatedly in the range of 2 to 50 times, or up to 100 times or more, at intervals in the range of 2 weeks up to 6 months apart. The intervals between the repeat administrations can be constant of vary between the respective doses of surface-engineered virus vaccine. Also contemplated herein, by virtue of the advantageous nature of the invention surface-engineered virus vaccines, is the substantially indefinite administration of the same, or substantially the same, invention surface-engineered virus vaccine bi-annually, annually, semi-annually, every 3-months, and the like; for the remainder of the respective individual's lifespan.

In yet further embodiment, the surface-engineered recombinant virus is a vaccine and is selected from AZD1222 (AstraZeneca), ChAdOx1-nCov19 (Oxford), Ad5-nCoV (CanSino), VSV, Ad26 (J&J), and the like.

In an embodiment, the surface-engineered recombinant virus is a vaccine and the coating of the virus increases humoral immunity induced by the virus against SARS-CoV-2 spike protein. Such increased humoral immunity may be characterized by increased IgG antibody levels.

In an embodiment, the surface-engineered recombinant virus is a vaccine and the coating of the virus increases cellular immunity induced by the virus against SARS-CoV-2 spike protein. Such increased cellular immunity may be characterized by increased CD8+ lymphocyte levels or activity. Such increased cellular immunity may be characterized by increased memory T cell levels or activity. Such increased cellular immunity may be characterized by increased memory T cell levels or activity.

In an embodiment, the surface-engineered recombinant virus is a vaccine, and the coating of the virus increases immunity induced by the virus against SARS-CoV-2 spike protein in an individual having pre-existing neutralizing antibodies against Ad5 adenovirus. Such increased immunity may be characterized by increased IgG antibody levels. Such increased cellular immunity may be characterized by increased CD8+ lymphocyte levels or activity. Such increased cellular immunity may be characterized by increased memory T cell levels or activity.

In another embodiment, the surface-engineered recombinant virus and/or recombinant viral genome is an adeno-associated virus of any known serotype, including but not limited to AAV1, 2, 3, 4, 5, 6, 7, 8, and 9. Because adeno-associated virus is a non-enveloped virus, the surface engineering of the present invention includes applying the inventive coating to the surface of the capsid of an adeno-associated virus using the methods described herein. Native AAV1, 2, 4, 5, 8, and 9 are known to infect central nervous system tissue. Native AAV1, 8, and 9 are known to infect heart tissue. Native AAV2 is known to infect kidney tissue. Native AAV7, 8, and 9 are known to infect liver tissue. Native AAV4, 5, 6, and 9 are known to infect lung tissue. Native AAV8 is known to infect pancreatic tissue. Native AAV2, 5, and 8 are known to infect photoreceptor cells. Native AAV1, 2, 4, 5, and 8 are known to infect retinal pigment epithelial tissue. Native AAV1, 6, 7, 8, and 9 are known to infect skeletal muscle tissue. In another embodiment, the surface-engineered recombinant virus is a pseudotyped adeno-associated virus, where the viral capsid and genome are from different viral serotypes. For example, AAV2/5 has the genome of serotype 2 packaged in the capsid from serotype 5. AAV2/5 targets neurons that are not efficiently transduced by AAV2/2, and is distributed more widely in the brain, indicating improved transduction efficiency. In another embodiment, the surface-engineered recombinant virus is an adeno-associated virus with a hybrid capsid derived from multiple different serotypes. One common example is AAV-DJ, which contains a hybrid capsid derived from eight serotypes. AAV-DJ displays a higher transduction efficiency in vitro than any wild type serotype; in vivo, it displays very high infectivity across a broad range of cell types. The mutant AAV-DJ8 displays the properties of AAV-DJ, but with enhanced brain uptake. The surface-engineered recombinant adeno-associated virus and/or viral genome of the present invention can be used deliver genes to any of the above tissues or cells, including in vivo. The surface-engineered recombinant adeno-associated virus and/or viral genome can be used to deliver genes to any tissue type known to those of skill the art to be targeted by native adeno-associated virus. Through engineering of the surface to contain appropriate ligands known to those of skill in the art, the surface-engineered recombinant adeno-associated virus and/or viral genome can be used to deliver genes to any cell or tissue type not normally targeted by the native adeno-associated virus. In another embodiment, the surface-engineered recombinant virus or viral genome is self-complementary AAV (scAAV). scAAV contains complementary sequences that are capable of spontaneously annealing, upon infection, which eliminates the requirement for host cell DNA synthesis. In another embodiment, the surface-engineered recombinant virus or viral genome has increased packaging capacity, for example through concatemer formation or homologous recombination as known to those of skill in the art.

The surface engineering of the recombinant adeno-associated virus and/or viral genome in any of the embodiments above can enhance in vivo transduction and/or enable repeated in vivo administration of the recombinant adeno-associated virus, including with systemic administration, because the coating of the virus protects the virus against clearance by the immune system and/or protects the virus from binding of neutralizing antibodies. Thus, the surface-engineered recombinant adeno-associated virus or viral genome can be used, including for systemic delivery, even when the recipient has pre-existing immunity and/or pre-existing antibodies against adeno-associated virus. The surface-engineered recombinant adeno-associated virus or viral genome can be used for multiple administrations to increase the amount and/or duration of transgene expression, even when the first administration of a native, non-surface-engineered recombinant adeno-associated virus would normally induce an immune response that limits the effectiveness of subsequent administrations. The surface-engineered recombinant adeno-associated virus and/or viral genome in any of the embodiments above can be used as a gene therapy or as a vaccine. The surface-engineered recombinant adeno-associated virus and/or viral genome in any of the embodiments above can be used as a vector to deliver genes encoding components for genome editing, such as Cas, TALEN, or zinc finger nucleases.

In yet further embodiments, the recombinant virus and/or recombinant viral genome is a gene therapy selected from Onasemnogene Abeparvovec-Xioi (AveXis/Novartis), Voretigene neparvovec-rzyl (Spark), MB-107 (Mustang Bio), AMT-061 (uniQure), PTC-AADC (PTC Therapeutics), ALD-104 or Starbeam ALD-102 (Bluebird Bio), VB-111 (Vascular Biogenics), EB-101 (Abeona Therapeutics), BIIB-111 (Biogen), BENEGENE-2 (Spark/Pfizer), BIIB112 (Biogen), SRP-9001 (Sarepta), BMN-270 (BioMarin), OXB-102 (Oxford BioSciences), HMI-102 (Homology Medicines), RP-A501 (Rocket Pharmaceuticals), LB-001 (LogicBio Therapeutics), Ad-RTS-hIL-12 (ZIOPHARM Oncology), SGT-001 (Solid Biosciences), B-VEC (Krystal Biotech), SRP-9003 (Sarepta), RG6357 (Roche/Spark), MYO-201 (Sarepta), RGX-314 (RegenXBio), AAV-GAD (MeiraGTx), MYO-102 (Sarepta), DTX401 (Ultragenyx), VY-AADC (Neurocrine/Voyager), AAV-AQP1 (MeiraGTx), EDIT-101 (Editas/Allergan), DTX301 (Ultragenyx), ADVM-022 (Adverum), RGX-111, RegenXBio, OXB-201 (Oxford BioMedica), AT132 (Axovant/Astellas), AVXS-201(AveXis/Novartis), ABO-102 (Abeona), ST-920 (Sangamo), AT-GTX-501 (Amicus), AT-GTX-502 (Amicus), ACHM-CNGB3 (Applied Genetic Technologies), AGTC-402 (Applied Genetic Technologies), AXO-AAV-GM1 (Axovant), AGTC-501 (Applied Genetic Technologies), ABO-101 (Abeona), SB-318 (Sangamo), AXO-AAV-GM2 (Axovant), AAV-RPE65 (MeiraGTx), RG6367 (Roche/Spark), RGX-121 (RegenXBio), RGX-501 (RegenXBio), RG6358 (Roche/Spark), MYO-301 (Sarepta), HMI-103 (Homology Medicines), LB-101 (LogicBio), HMI-202 (Homology Medicines), AVR-RD-03 (Avrobio), MYO-103 (Sarepta), and BBP-631 (BridgeBio Pharma).

One aspect of the invention provides a method of administering a gene therapy to an individual in need thereof, comprising administering to the individual a surface-engineered recombinant gene-therapy virus that is a gene therapy; thereby administering the gene therapy to the individual. The surface-engineered recombinant gene-therapy virus can be any be of those described herein. The surface-engineered recombinant gene-therapy virus can be administered by intramuscular, intravenous, intracranial, or intrathecal injection, or injection into any tissue where transgene expression is desired. In one embodiment, the surface-engineered gene-therapy virus is administered once as a single dose. In this embodiment, there is no need for repeat administration as the level of therapy provided is durable for the lifespan of the individual.

As used herein, the phrase “gene therapy” refers to the administration of a transgene to an individual such that a therapeutic benefit is bestowed upon that individual. In one embodiment, the gene therapy can be the replacement of a defective gene or cDNA coding region encoding a non-functional or sub-optimally functional protein, with a non-defective gene or cDNA coding region, such that a functional protein is expressed in the individual being treated.

In other embodiments of the methods of administering a gene therapy, the same surface-engineered gene-therapy virus is administered repeatedly at 2 or more intervals. For example, an invention surface-engineered gene-therapy virus can be administered repeatedly at least twice at intervals in range of 1 week up to 3 or more years apart. In one embodiment, an invention surface-engineered gene-therapy virus is administered repeatedly in the range of 2 to 50 times, or up to 100 times or more; at intervals in the range of 2 weeks up to 6 months, 1 year, or 2 years apart. The intervals between the repeat administrations can be constant of vary between the respective doses of surface-engineered gene-therapy virus. Also contemplated herein, by virtue of the advantageous nature of the invention surface-engineered gene-therapy virus, is the substantially indefinite administration of the same, or substantially the same, invention surface-engineered gene-therapy virus bi-annually, annually, semi-annually, every 3-months, and the like; for the remainder of the respective individual's lifespan.

Exemplary enveloped viruses or lipid containing viruses contemplated for use as recombinant viruses herein include: Poxvirus (e.g., vaccinia virus, and the like), Herpes Virus (e.g., herpes simplex, and the like), Rhabdovirus (e.g., Vesicular stomatitis virus, and the like), Coronavirus (e.g., SARS-CoV-2, and the like), Hepadnaviruses, Asfarviridae, Flavivirus, Alphavirus, Togavirus, Hepatitis D, Orthomyxovirus, Paramyxovirus, Bunyavirus, Filovirus, Retroviruses, Alphavirus (alphaviruses), Rubivurus (rubella virus), Flavivirus (Flaviviruses), Pestivirus (mucosal disease viruses), hepatitis C virus, Coronavirus, (Coronaviruses), severe acute respiratory syndrome (SARS), Torovirus, (toroviruses), Arteivirus, (arteriviruses), Paramyxovirus, (Paramyxoviruses), Rubulavirus (rubulavriuses), Morbillivirus (morbillivuruses), Pneumovirinae (the pneumoviruses), Pneumovirus (pneumoviruses), Vesiculovirus (vesiculoviruses), Lyssavirus (lyssaviruses), Ephemerovirus (ephemeroviruses), Cytorhabdovirus (plant rhabdovirus group A), Nucleorhabdovirus (plant rhabdovirus group B), Filovirus (filoviruses), Influenzavirus A, B (influenza A and B viruses), Influenza virus C (influenza C virus), (unnamed, Thogoto-like viruses), Bunyavirus (bunyaviruses), Phlebovirus (phleboviruses), Nairovirus (nairoviruses), Hantavirus (hantaviruses), Tospovirus (tospoviruses), Arenavirus (arenaviruses), unnamed mammalian type B retroviruses, unnamed, mammalian and reptilian type C retroviruses, unnamed, type D retroviruses, Lentivirus (lentiviruses), Spumavirus (spumaviruses), Orthohepadnavirus (hepadnaviruses of mammals), Avihepadnavirus (hepadnaviruses of birds), Simplexvirus (simplexviruses), Varicellovirus (varicelloviruses), Betaherpesvirinae (the cytomegaloviruses), Cytomegalovirus (cytomegaloviruses), Muromegalovirus (murine cytomegaloviruses), Roseolovirus (human herpes virus 6, 7, 8), Gammaherpesvirinae (the lymphocyte-associated herpes viruses), Lymphocryptovirus (Epstein-Barr-like viruses), Rhadinovirus (saimiri-ateles-like herpes viruses), Orthopoxvirus (orthopoxviruses), Parapoxvirus (parapoxviruses), Avipoxvirus (fowlpox viruses), Capripoxvirus (sheeppox-like viruses), Leporipoxvirus (myxomaviruses), Suipoxvirus (swine-pox viruses), Molluscipoxvirus (molluscum contagiosum viruses), Yatapoxvirus (yabapox and tanapox viruses), Unnamed, African swine fever-like viruses, Iridovirus (small iridescent insect viruses), Ranavirus (front iridoviruses), Lymphocystivirus (lymphocystis viruses of fish), Togaviridae, Flaviviridae, Coronaviridae, Enabdoviridae, Filoviridae, Paramyxoviridae, Orthomyxoviridae, Bunyaviridae, Arenaviridae, Retroviridae, Hepadnaviridae, Herpesviridae, Poxviridae, and any other lipid-containing, enveloped virus. In certain embodiments, an enveloped recombinant virus has had its native-envelope removed prior to coating with the artificial coating layer.

Exemplary natively non-enveloped viruses contemplated for use as recombinant viruses herein include adeno-associated viruses, adenoviruses, noroviruses, rhinoviruses, polioviruses, coxsackieviruses, rotaviruses, hepatitis A virus, flock house virus, reoviruses, and papillomaviruses.

In another embodiment, the surface-engineered recombinant virus is a vaccine and the virus is a replication deficient Ad5 (Human) Adenovirus vector, wherein the recombinant genome encodes SARS-CoV-2 spike protein and the E1 & E3 genes are deleted, or the recombinant genome does not contain any native Ad5 viral genes. In yet another embodiment, the surface-engineered recombinant virus is oncolytic and the virus is VSV.

In yet further embodiments, the payload is a nucleic acid, and the nucleic acid is selected from that contained in QR-110 (ProQR therapeutics), Neovasculgen (Human Stem Cells Institute), ND-L02-s0201 (Nitto Denko/BMS), HGF plasmid (AnGes), TAVO (Oncosec), GEN-1 (Celsion), QR-313 (Wings Therapeutics), INXN-4001 (Triple-Gene), AAT genome editing (Intellia), QR-411 (ProQR Therapeutics), patisiran, givosiran, lumasiran, vultisiran, cemdisiran, inclisiran, ALN-AAT02, ALN-AGT, ALN-HSD (Alnylam), ALN-COV, ALN-HBV02 (Alnylam/Vir), BNT162b2 (Pfizer-BioNTech), mRNA-1273, mRNA-1647, mRNA-1653, mRNA-1893, mRNA-1345, mRNA-1189, mRNA-1010, mRNA-1020, mRNA-1030, mRNA-1644, mRNA-1574, mRNA-1215, mRNA-1851, mRNA-1944, mRNA-0184, mRNA-6981, mRNA-6231, mRNA-4157, mRNA-5671, mRNA-2416, mRNA-2752, MEDI1191, AZD8601, mRNA-3927, mRNA-3705, mRNA-3283, mRNA-3745 (Moderna), MRT5005, MRT5500 (TranslateBio). In certain embodiments, the payload is mRNA, and the mRNA encodes patient-specific neoantigens to enable personalized anti-cancer therapy.

As used herein, the phrase “artificial coating layer” or “OnCoat” refers to any biocompatible material that functions to encapsulate or surround the payload, i.e., the recombinant virus, viral capsid, or nucleic acid. In each of the invention coated delivery systems provided herein, the artificial coating-layer protects the recombinant virus or the previously-enveloped-capsid or the nucleic acid from immune recognition and neutralization during therapy. In particular embodiments, the artificial coating is selected from the group consisting of: silica, titanium oxide and calcium phosphate. In certain embodiments, the artificial coating is applied by conducting a charge-mediated sol-gel condensation reaction directly onto the surface of the recombinant virus. In further embodiments, the artificial coating comprises a silica gel matrix or titanium oxide gel matrix encapsulating the recombinant virus.

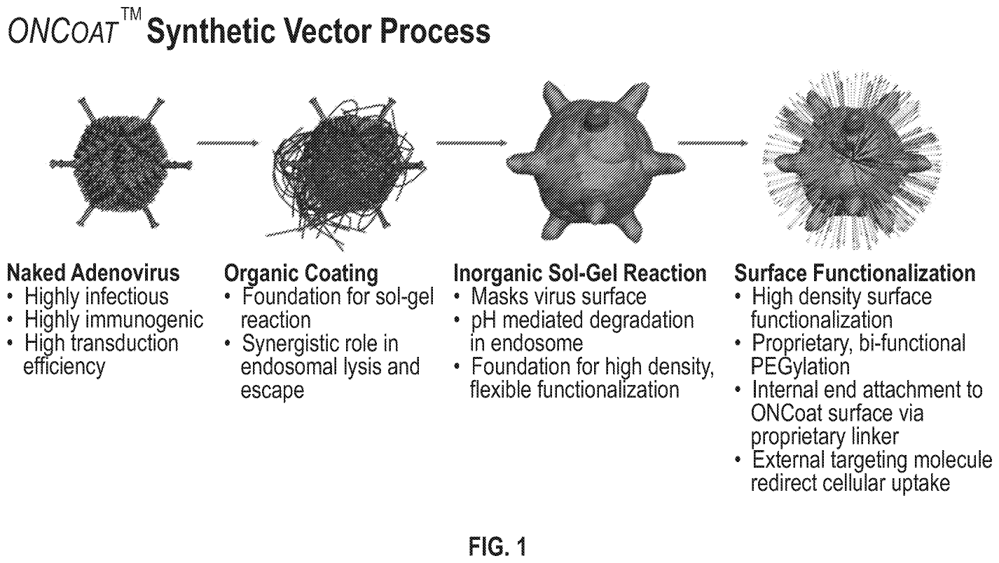

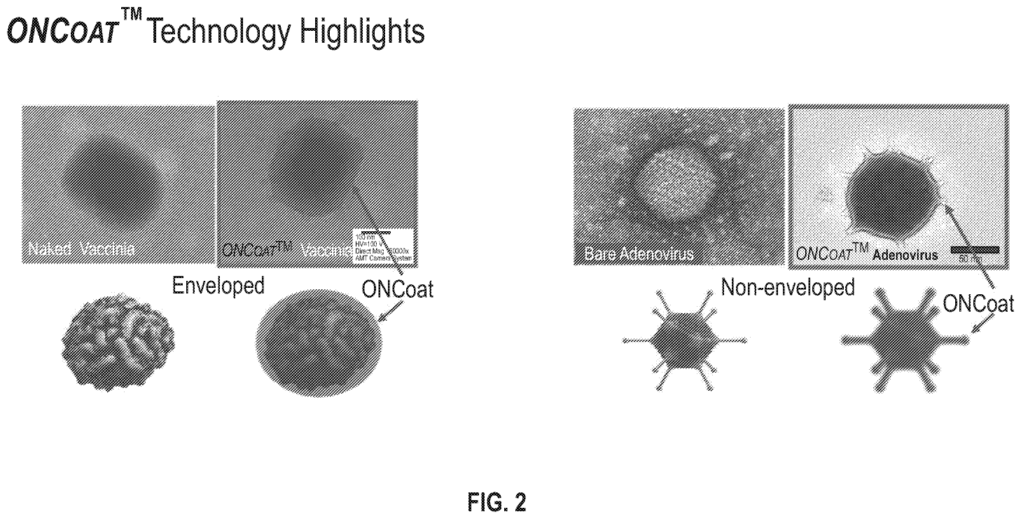

In particular embodiments for applying the artificial coating layer, as set forth in the methods described in US 2019/0151253A1, the virus or viral capsid is incubated with polycationic polymer to shift the virus surface charge towards the positive direction. Next, a silica precursor, such as tetramethyl (or tetraethyl) orthosilicate, and the like, is hydrolyzed to generate silicic acid, which is added into a suspension containing the virus or viral capsid surface-modified with polycationic polymer. The polymer on the virus surface templates the polycondensation reaction (sol-gel) to produce silica gel. In particular embodiments, the surface can then further functionalized with stabilization and targeting ligands. The scheme for applying the coating layer and stabilization and targeting ligands is shown in FIG. 1. FIG. 2 shows electron microscope images of surface-engineered vaccinia virus and adenovirus generated using the inventive coating.

In the case of a nucleic acid, the nucleic acid can be complexed with a cation such as a polycationic polymer or cationic lipid to form a positively charged polyion complex. Methods for such complexation are described, for example, by de Ilarduya (Eur J Pharm Sci. 2010 Jun. 14; 40(3):159-70) and are well known to those of skill in the art. The silica precursor is then added to a suspension containing the polyion complex containing the nucleic acid, and the surface of the polyion complex templates the polycondensation reaction (sol-gel) to produce silica gel. In particular embodiments, the surface can then further functionalized with stabilization, targeting, an uptake-enhancing ligands.

The polycationic polymer used for any embodiment herein can be any biomedically suitable polycationic polymer known to those of skill in the art to electrostatically interact with negatively charged molecules or surfaces, including but not limited to poly-L-lysine (PLL), polyarginine, polyethyleneimine (PEI), polyallylamine, polyamines, diethylaminoethyl-dextran (DEAE-dextran), branched polymers such as poly(amidoamine) (PAMAM) dendrimers, Tfx-50, dioctadecylamidoglycylspermine, or positively charged polypeptides. The cationic lipid can be any biomedically suitable cationic lipid polycationic polymer known to those of skill in the art to electrostatically interact with negatively charged molecules or surfaces such as MVLS, DOTMA, ethyl PC, DDAB, DOTAP, DC-cholesterol, GL67, or DODMA. Any suitable combination of cationic polymers can be used. Any suitable combination of cationic lipids can be used. Any suitable combination of cationic polymers and lipids can be used.

The artificial coating used for any embodiment herein is preferably silica, titanium oxide, or calcium phosphate. The artificial coating is particularly preferably formed from a silica gel, titanium oxide gel, or calcium phosphate gel formed through a sol-gel condensation reaction. The calcium phosphate can be hydroxyapatite, tricalcium phosphate, biphasic calcium phosphate or any other suitable calcium phosphate. However, the artificial coating is not limited to these materials, and any other material known to those of skill in the art to form gels through sol-gel condensation reactions and to be suitable for biomedical use can be used as the artificial coating. Zinc oxide, magnesium oxide, calcium oxide, zirconium oxide, aluminum oxide, iron oxide, tungsten oxide, cerium oxide, tin oxide, or any other suitable metal oxide can also be the artificial coating material. Any combination of suitable materials can also be used as the coating material.

In some embodiments, for example, an exemplary silica-based or titanium oxide-based artificial coating-layer can be formed by the direct condensation of a removable silica matrix or titanium oxide matrix on the surface of the recombinant viruses or previously-enveloped-viral capsids to form the surface-engineered-recombinant virus while preserving biological activity. Using the disclosed fabrication techniques, for example, the exemplary silica matrix or titanium oxide matrix can be formed directly on the surface of the recombinant viruses or previously-enveloped-capsids under suitable reaction conditions. This allows higher encapsulation efficiency without consequent loss of activity of the recombinant virus or viral capsids. This also brings fine control over particle size giving surface-engineered-recombinant viruses with well-defined size characteristics. For example, silica or titanium oxide can be used to form the artificial coating-layer due to its biocompatibility and biodegradability.

In other embodiments, the disclosed technology can include a sensitizing agent within the synthesized surface-engineered delivery system to allow externally triggered release of the encapsulated payload. The sensitizing agent can be fluorocarbon emulsions as ultrasound cavitation centers. The sensitizing agent can be pH-responsive. Also provided herein are exemplary methods for the surface-functionalization of the artificial-coating-layer for, e.g., functionalizing of the silica or titanium surface to improve circulation time (increase biological half-life), tumor and/or antigen targeting, cell/tissue targeting, cell uptake and the like.

In another embodiment, the exemplary method for the encapsulation process of a recombinant virus or a previously-enveloped-capsid that preserves the gene transcription activity of the encapsulated recombinant virus or engineered-naked-capsids includes forming an intermediate biomaterial by binding a surface-charged material with a naked-viral-capsid from a non-enveloped virus or a previously-enveloped-capsid, such that the formed intermediate biomaterial comprises regions having a net surface charge, e.g., which may be different from the original surface charge on the capsid. For example, in a particular embodiment, a negatively charged naked-viral-capsid (e.g., from a non-enveloped virus or a previously-enveloped-capsid) is electrostatically reacted with a cationic polymer, poly-L-lysine (PLL), to modify the surface charge on the viral-capsid, e.g., the PLL is bound to the surface of the naked-viral-capsid by an electrostatic force. The invention method includes forming a surface-engineered-recombinant virus by forming an artificial coating-layer to encapsulate the intermediate biomaterial (e.g., the cationic polymer and PLL), in which the encapsulated viral-capsid maintains its ability to regulate gene expression (e.g., via gene transcription). For example, in one embodiment where silica is utilized, the exemplary positively charged viral-material structure attracts negatively charged silica precursor and hydroxyl ions creating a basic environment suitable for a silica polycondensation reaction to form the viral-capsid coating-layer (e.g., silica-based nanoparticle) that encapsulates the viral capsid. The poly-L-lysine can be of any molecular weight, or it can have a molecular weight of 30-70 kDa. It was unexpectedly found that when the molecular weight of the poly-L-lysine was 30-70 kDa, entry of the payload, e.g., the virus, into the cell nucleus was enhanced. The presence of poly-L-lysine with a molecular weight of 30-70 kDa may facilitate endosomal release and/or nuclear trafficking of the payload.

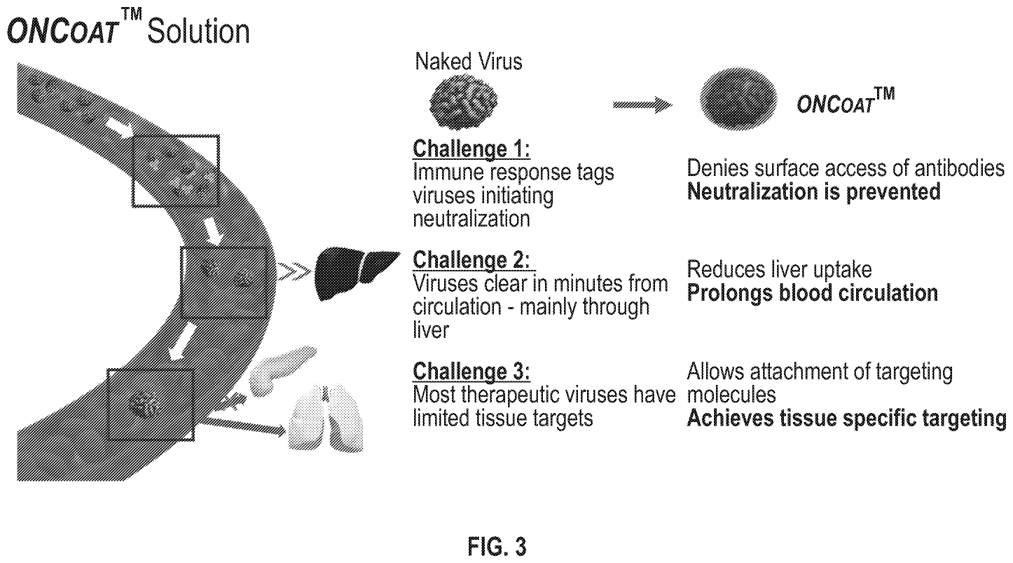

The surface of the surface-engineered recombinant virus or viral genome can be readily functionalized with ligands and other molecules to impart new properties to the surface-engineered recombinant virus or viral genome. In an embodiment, the functional molecule is attached to the surface of the surface-engineered recombinant virus or viral genome using a bifunctional spacer molecule with an anchor at one end for anchoring to the surface of the surface-engineered recombinant virus or viral genome, and a functional molecule such as a ligand at the other end. In an embodiment, the spacer is PEG, and the anchor is silane. One end of the PEG is functionalized with silane and the other is functionalized with the ligand. For attachment to the surface of the surface-engineered recombinant virus or viral genome, the silane in the ligand-PEG-silane molecule is hydrolyzed, and the ligand-PEG-silane molecule is then mixed with the surface-engineered recombinant virus or viral genome. The hydrolyzed silane attaches to the coating material on the surface of the surface-engineered recombinant virus or viral genome, such that it anchors the ligand-PEG-silane molecule to the surface of the surface-engineered recombinant virus or viral genome and displays the ligand on the surface. This functionalization imparts the properties of the ligand to the surface-engineered recombinant virus or viral genome. For example, in an embodiment where the ligand is folate, attachment of folate-PEG-silane to the surface of the surface-engineered recombinant virus or viral genome allows targeting of cancer cells. FIG. 3 shows the benefits provided by the surface engineering, including preventing neutralization, reducing liver uptake and clearance, and improving tissue targeting.

The use of silane chemistry for attachment of ligands and other functional molecules to the surface of the surface-engineered recombinant virus or viral genome is advantageous because it is simple and easily performed, and silane provides easy and biocompatible attachment that is stable in physiological conditions. The attachment reaction can be performed in physiological buffered conditions in a matter of minutes to a few hours with very high density, without negatively affecting the biological activity of the payload or virus. The ease of the attachment reaction also allows easy interchangeability of ligands during manufacturing; stocks of different ligand-PEG-silane molecules can be kept and used to impart different properties to the surface-engineered recombinant virus or viral genome as needed.

In addition to silane, any anchor molecule or moiety reasonably expected to attach to the coating material can be used to anchor the functional molecule to the surface of the surface-engineered recombinant virus or viral genome. Such an anchor can attach to the coating material, and thereby attach to the surface of the surface-engineered recombinant virus or viral genome, using ionic bonds, electrostatic interactions, covalent bonds, hydrogen bonds, van der Waals interactions, metallic bonds, physical interactions, precipitation, or any other kind of bond or interaction known by those of skill in the art to attach molecules to the materials used in the coating. Combinations of different anchors can be used. Such anchors and their use to attach molecules to substrates are well known to those of skill in the art. Such anchors include, but are not limited to, amino acids such as L-arginine and L-lysine; amine or thiol groups introduced to the silica coating; and siloxanes, silanols, alkoxysilanes, aminosilanes, or chlorosilanes. These anchors are commonly used to functionalize silica and their use is known to those of skill in the art; other anchors known in the art to functionalize silica can also be used when the coating is silica. Additionally, any reaction capable of reacting with free silyl hydroxide moieties present or introduced in the surface of the silica coating may be used to covalently modify the surface. For example, the surface of the silica coating may be treated with a trialkoxysilyl compound or trihydroxysilyl compound. The compound reacts with the silyl hydroxide surface of the silica body, forming covalent silicon-oxygen bonds. Trialkoxysilyl and trihydroxysilyl compounds may be used to modify the surface of the silica coating. The anchor can be trihydroxysilyl propyl methylphosphonate. Anchors for covalent bonding can be amine-NETS (N-hydroxysuccinimide), carboxylate-1-ethyl-3-(3-dimethyl amonipropyl) carbodiimide (EDC)-amine, carboxylate-EDC+NETS-amine, amine/sulfhydryl-epoxide, amine-isothiocyanate, amine-azlactone, amine-p-nitrophenyl ester, amine-tyrosinase (TR)-tyrosine, sulfhydryl-maleimide, reactive hydrogen-benzophenone. These covalent anchors can also be used when amine groups are introduced into the silica coating. An enormous variety of covalent conjugation chemistries beyond those listed here are known to those of skill in the art. See, for example Kim et al. Biomicrofluidics 7, 041501 (2013), Rusmini et al. Biomacromolecules 8, 1775 (2007), and Hermansson Bioconjugate Techniques, 2nd ed. (Academic Press, San Diego, 2008), all incorporated herein by reference. These types of covalent conjugation chemistries can also be used to conjugate the functional molecule to the PEG.

The PEG used as a spacer molecule to connect the silane anchor with the functional molecule can be of any molecular weight appropriate for PEG spacers used to functionalize nanoparticles or microparticles. The PEG can have a molecular weight of 200 Da to 30 KDa or more. The PEG can have a molecular weight of 5 KDa, 10 KDa, or 20 KDa. The attachment of the PEG to the surface of the surface-engineered recombinant virus or viral genome using the anchor results in a PEG layer over the surface that provides additional protection from neutralization by antibodies as well as from opsonization. This PEG layer supplements the inventive coating's functions of preventing antibodies and other proteins or macromolecules from accessing the surface of the recombinant virus and preventing immune recognition of the virus. The protection provided by the PEG layer and inventive coating also improves the stability in serum/blood conditions and provides longer circulation and effective tissue accumulation. As is well known to those of skill in the art, tissue accumulation can be enhanced by the EPR effect, particularly in cancer. By using bifunctional PEG as a spacer in this manner, a single molecule with both stability and targeting capability can be obtained with a single-step reaction.

As an alternative to PEG, any spacer molecule used by those of skill in the art, including any bifunctional spacer used to connect ligands and substrates, can be used. Such spacer molecules are well known to those of skill in the art. The spacer can be a C1 to C12 alkyl chain. In other words, a C1 to C12 alkyl group is present between the atom covalently bonded to the surface of the coating and the functional molecule to be anchored to the surface of the coating. In other embodiments, the functional molecule is covalently bonded to the silica surface via a C1 to C6 alkyl linker. As used herein a C1 to C12 alkyl chain includes linear, branched and cyclic structures having 1 to 12 carbon atoms, and hybrids thereof, such as cycloalkylalkyl. Examples of alkyl chains include methylene (CH2), ethylene (CH2CH2), propylene (CH2CH2CH2), and so forth. The spacer molecule can be a chain formed from dialdehyde molecules, anhydride molecules, dichloride molecules, epihalohydrin molecules, or diepoxide molecules. The spacer can be epichlorohydrin, epibromohydrin, or epifluorohydrin. The spacer can be a water-soluble polymer, a nucleic acid, a polypeptide, an oligosaccharide, a carbohydrate, a lipid, or an ethylglycol. Combinations of different spacer molecules can be used, or the spacer molecule can be excluded, with the ligand directly attached to the anchor material anchoring the ligand to the coating over the recombinant virus or viral genome.

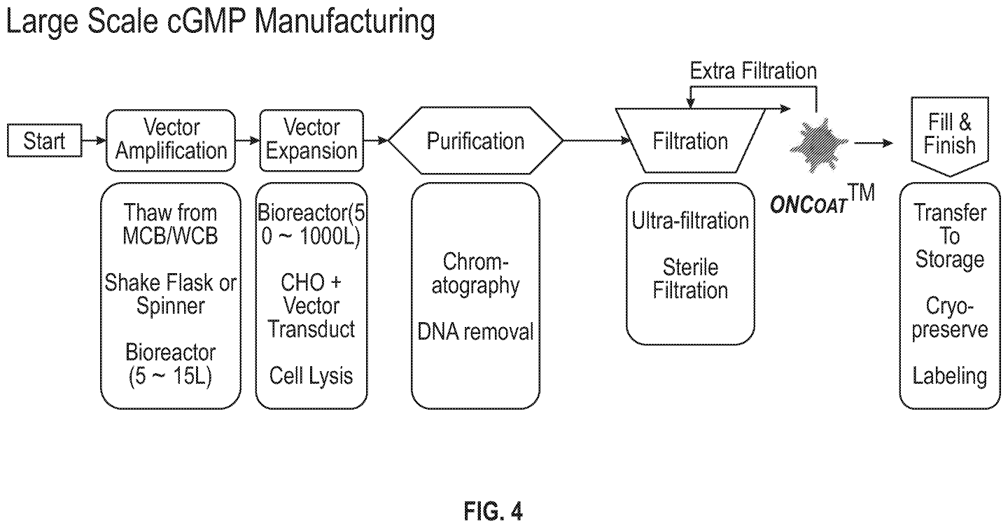

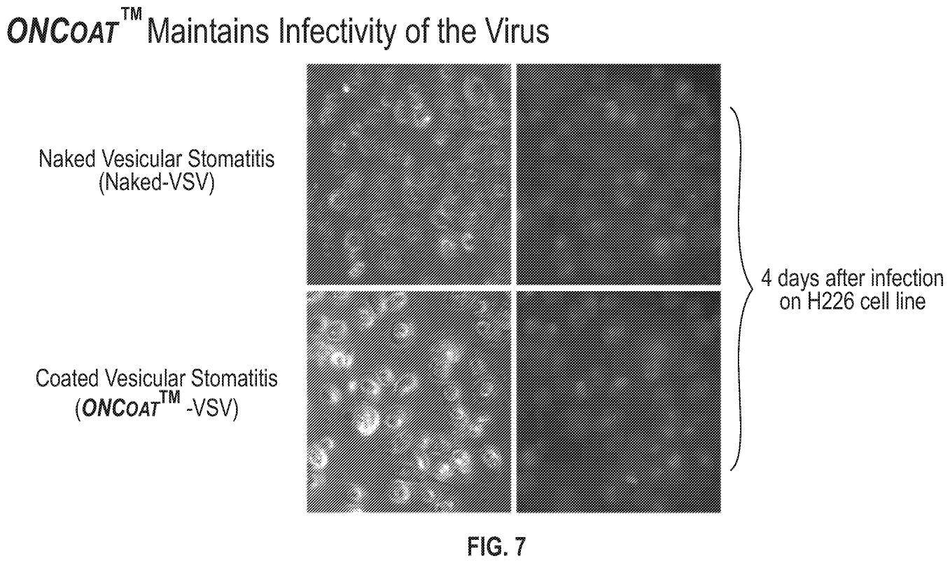

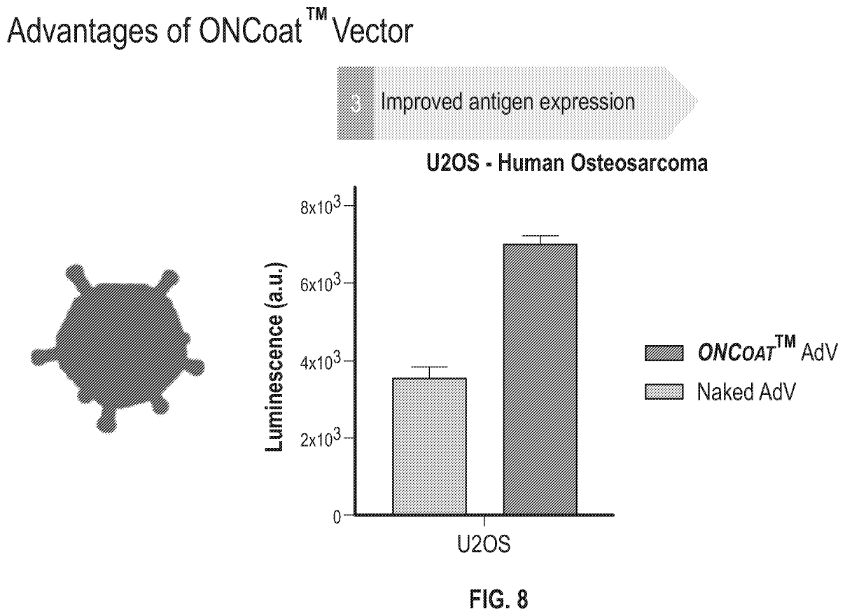

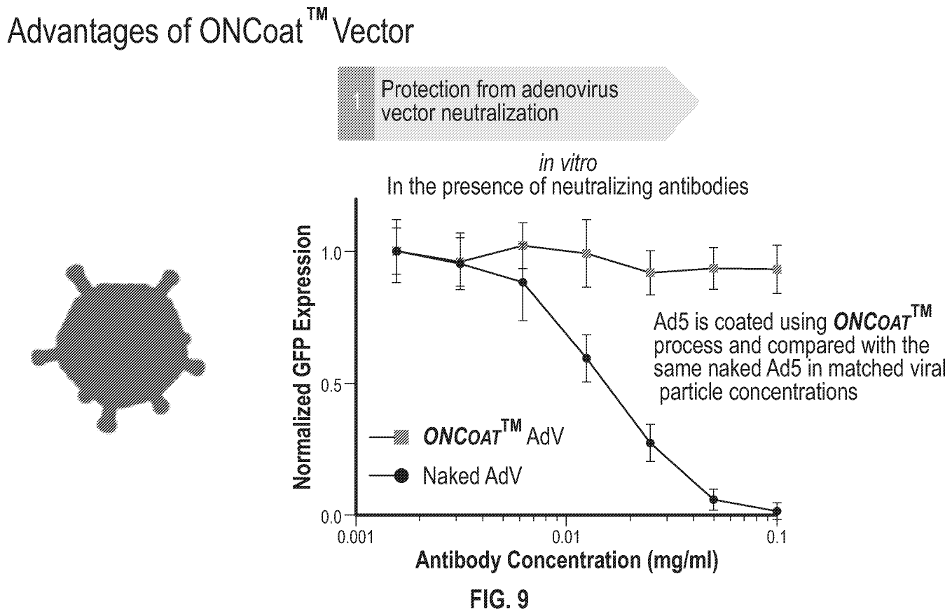

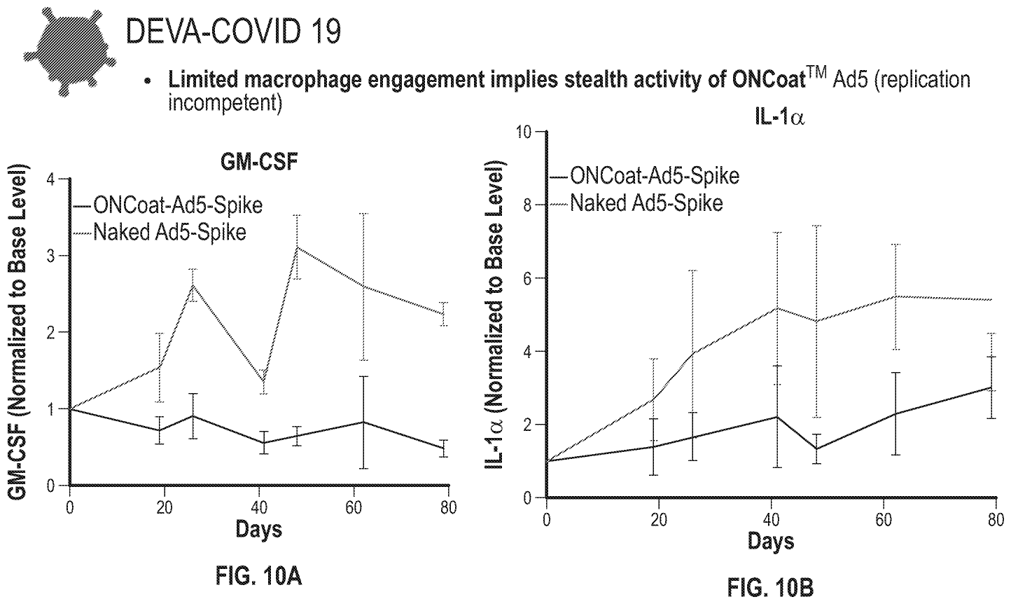

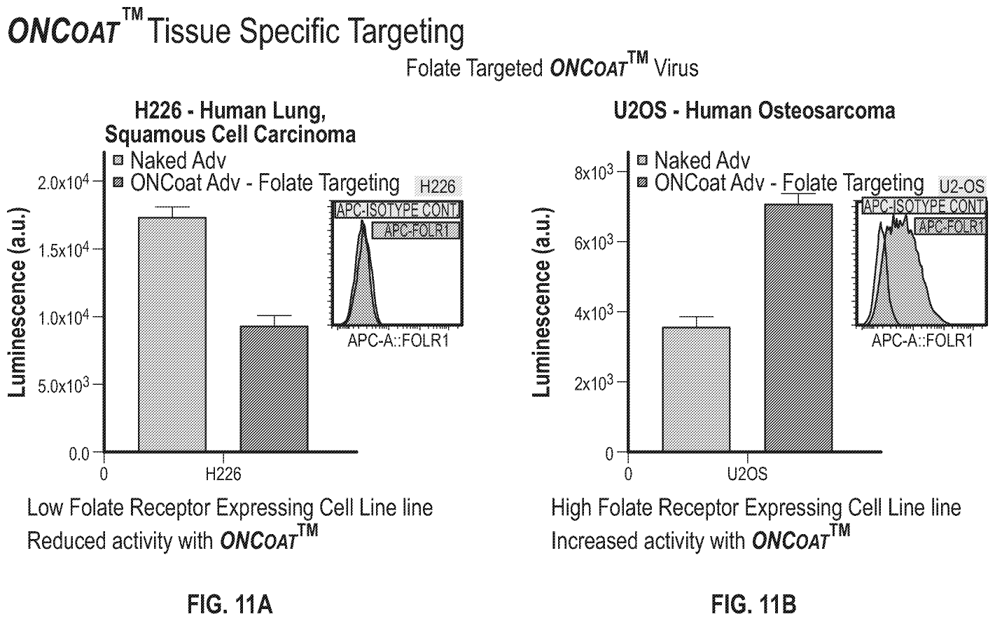

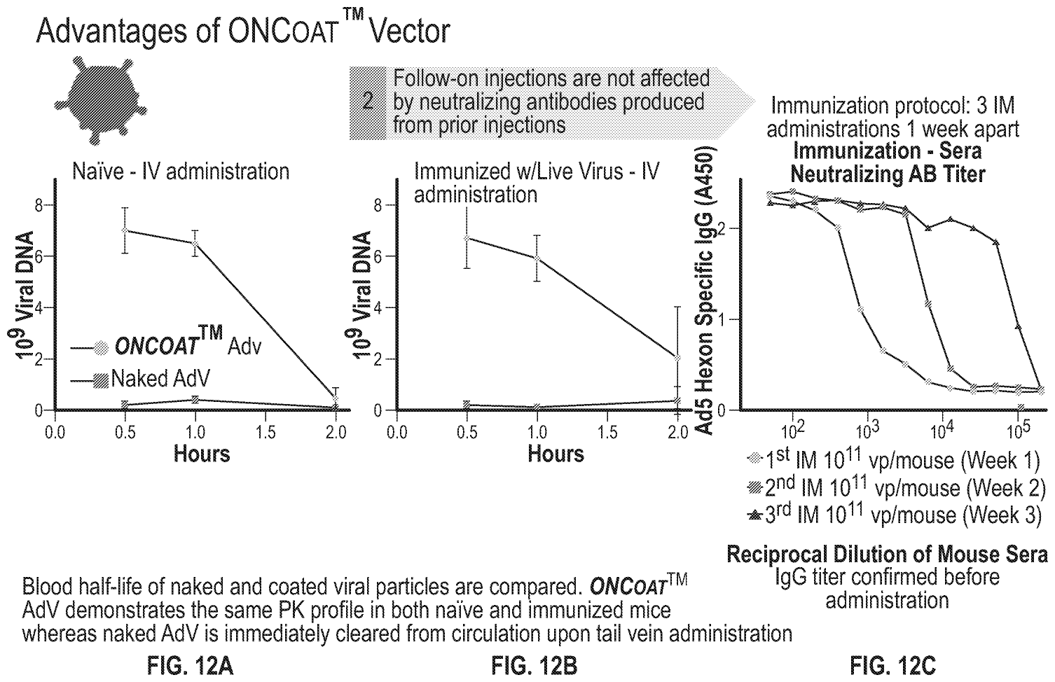

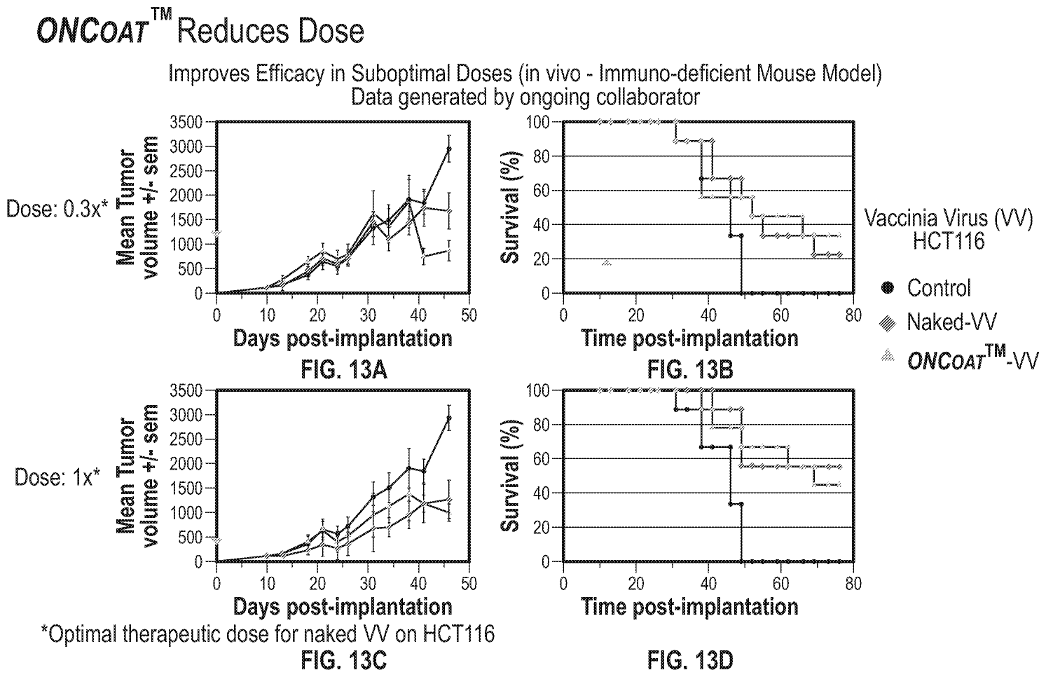

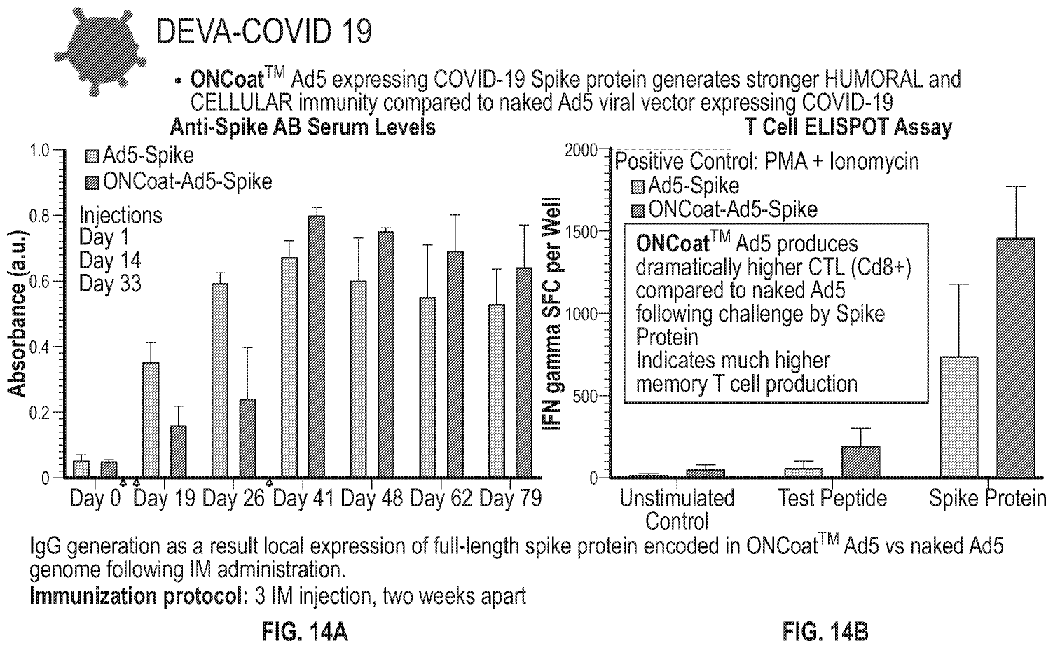

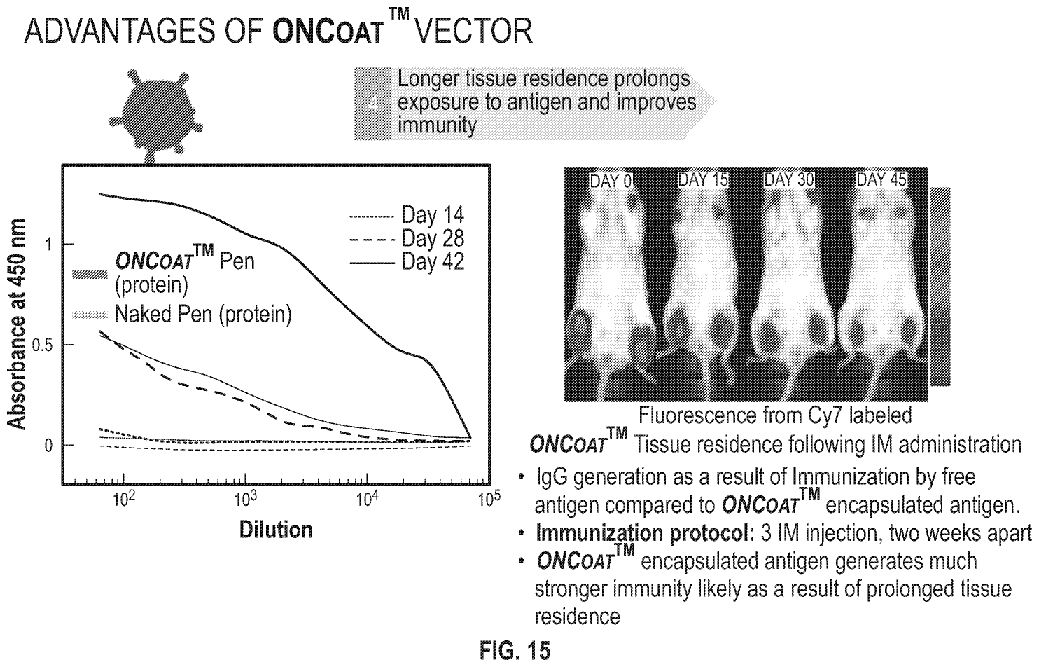

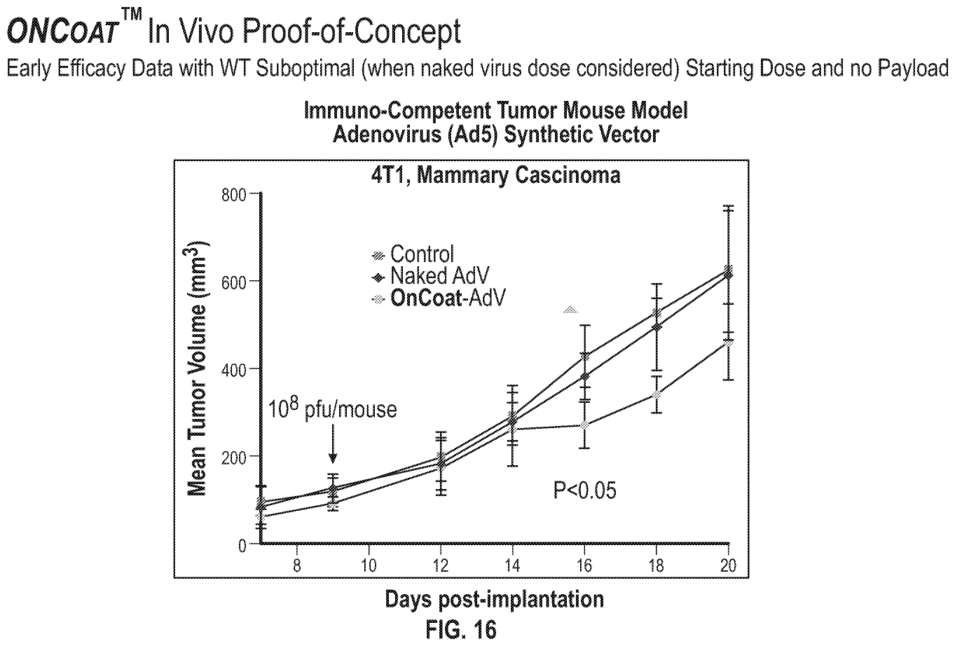

The self-assembling and self-terminating nature of the coating and ligand-anchoring processes, together with the fact that these processes are performed at room temperature and neutral pH in one-step reactions in bulk in aqueous conditions in a matter of minutes to hours, means that these processes can be easily scaled for large-scale GMP-grade manufacturing. The manufacturing scheme is shown in FIG. 4. The gentle and inert reaction conditions and nature of the reaction mean that the coating and ligand-anchoring processes do not damage the virus or negatively affect its infectivity. FIG. 7 shows that the coating does not negatively affect the infectivity.

The coating, with or without attached PEG, prevents or reduces toxicity of the recombinant virus upon administration. Lack of toxicity can be determined by normal levels of biomarkers where abnormal levels would be indicative of toxicity. Such biomarkers are well known to those of ordinary skill in the art. Lack of liver toxicity can be determined by normal aspartate transaminase (AST) and normal alanine transaminase (ALT) activity. In experiments conducted by the inventor, after three injections of 5×106 pfu/mouse (injections 2 days apart, livers harvested right after last injection), AST activity was 29+/−5 U/L (Normal: 25-100 U/L) and ALT activity was 27+/−12 U/L (Normal: 25-60 U/L).

The coating, with or without attached PEG, provides a “stealth” property such that it prevents or reduces recognition by macrophages and/or activation of macrophages. This helps to increase the circulation time and permit systemic administration and repeated administration. It may also help to reduce toxicity.

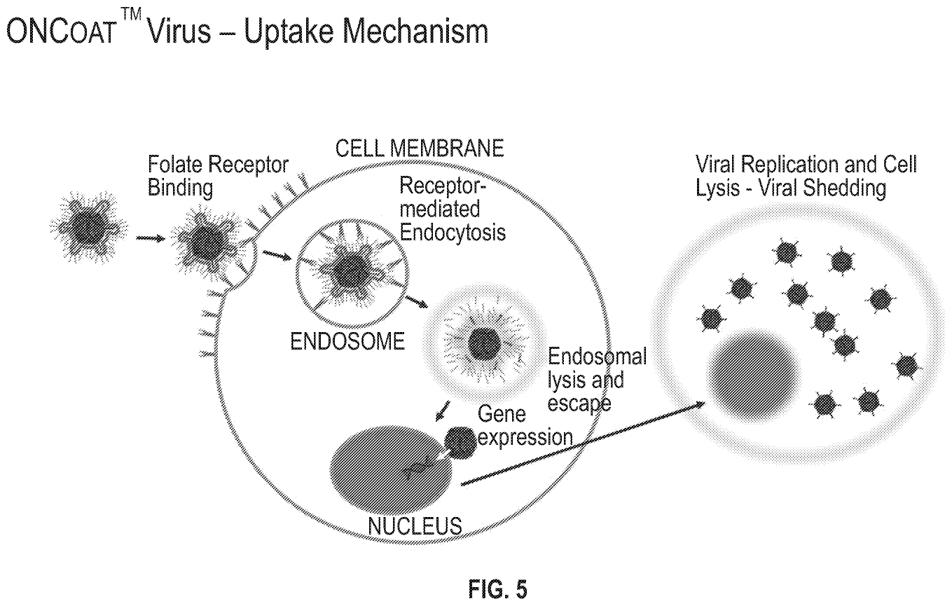

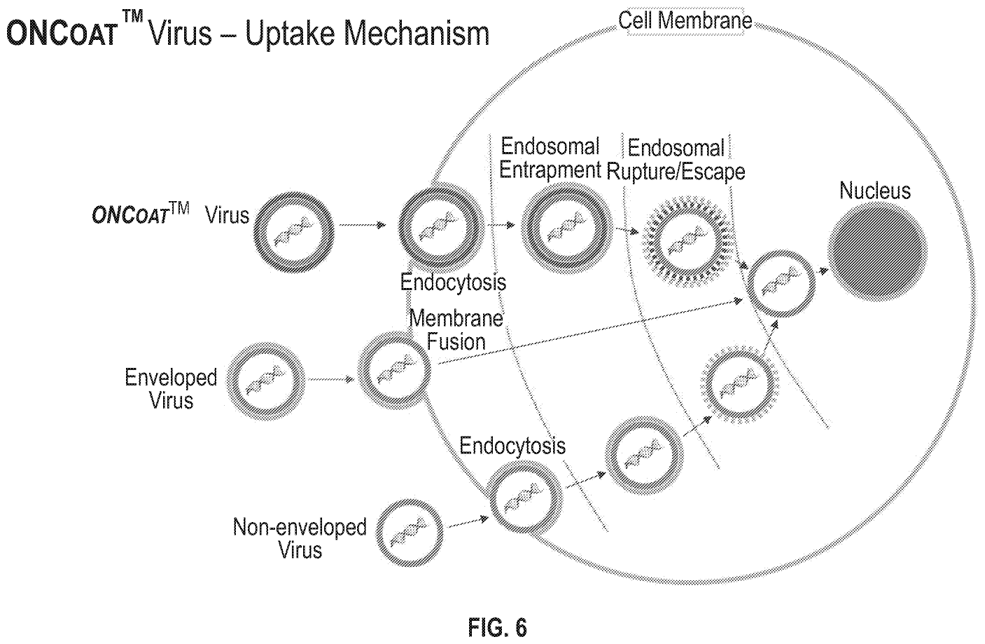

Folate is an exemplary functional molecule used to allow targeting of the surface-engineered recombinant virus or viral genome to cancer cells, such that the folate is attached to the coating over the surface-engineered recombinant virus or viral genome using a silane anchor with a PEG spacer. Folate-functionalized surface-engineered recombinant virus or viral genome can enter cells via receptor-mediated uptake, followed by accumulation in endosomes. In particular, in the late endosomal stage, when the pH inside the endosome decreases to approximately pH 5, the coating on the surface-engineered recombinant virus or viral genome degrades, releasing silicic acid and revealing the cationic polymer on the virus surface. This process eventually leads to rupture of the endosome, releasing the viral capsid into the cytoplasm. In another embodiment, the viral capsid is released in the endosome, escaping the endosome using its own machinery based on features of the capsid/core. When the viral capsid is released into the cytoplasm it can reach the nucleus and release its genomic payload into the nucleus using its natural processes as they are not negatively affected by the coating process used for surface engineering. In the case of RNA viruses, release into the cytoplasm will trigger expression of the genetic payload without need for delivering the payload into the nucleus. The coating with surface functionalization provides a shuttle for the recombinant virus payload through the circulation and targeted tissue into the cell. Inside the cell, the degradation of the coating allows the virus to complete its life cycle effectively. The uptake mechanism in shown in FIG. 5 and FIG. 6.

In addition to folate, any molecule known to have a relevant function can be similarly attached to the surface of the surface-engineered recombinant virus or viral genome, i.e., to the coating. For example, such molecules can allow targeting of the surface-engineered recombinant virus or viral genome to a particular cell, tissue, or organ. Such molecules can allow the surface-engineered recombinant virus or viral genome to cross biological barriers, such as blood vessel walls or the blood-brain barrier. Such molecules can facilitate uptake of the surface-engineered recombinant virus or viral genome into cells. Such molecules can induce endosomolysis or allow intracellular trafficking of the surface-engineered recombinant virus or viral genome to particular intracellular organelles such as the nucleus or mitochondria. Such molecules can allow the surface-engineered recombinant virus or viral genome to attach to a substrate such a biomaterial matrix to allow controlled release of the surface-engineered recombinant virus or viral genome. Such molecules can allow the surface-engineered recombinant virus or viral genome to attach to a substrate such as an affinity column or magnetic beads for isolation or purification. Such molecules can provide an immunostimulatory or immunomodulatory effect. Such molecules can have a pharmaceutical effect, for example blocking ion channels. Such molecules can be magnetic or susceptible to magnetic fields, such that a magnetic field can then be used to guide the surface-engineered recombinant virus or viral genome to a desired place in the body. Such molecules are well known to those of skill in the art. Such molecules can be proteins, peptides, polysaccharides, carbohydrates, lipids, fatty acids, synthetic or natural polymers, small molecules, RNA, DNA, aptamers, antibodies, antibody fragments, antigens, epitopes, cytokines, fluorescent markers, and/or growth factors. Examples of such functional molecules include, but are not limited to, antibodies or ligands that target the estrogen receptor, epidermal growth factor receptor, CD47, HER2 receptor, IL-4 receptor, AXL, ALK, PTK7, TM4F1, nectin4, PSMA, VEGFR, CTLA4, ERB22, CD20, CD22, CD30, CD33, CD52, CD74, CD276, and/or CD79. Such functional molecules also include TAT peptide, SV40 large T antigen, nuclear localization signal (NLS) peptides, cell penetrating peptides, and/or endosomolytic peptides such as melittin. Such functional molecules also include PECAM, transferrin, melanotransferrin, alanine, glutathione, OX26, and antibodies and ligands targeting GLUT-1, LRP1, TfR1, and/or SLC7A5 receptor. Such functional molecules also include hyaluronic acid, RGD peptide, Tyr3-octreotide, PE-221, octreotide, rabies virus glycoprotein-29, miniAp-4, angiopep-1, iRGD (CEND-1), BT1718, PL1, CARG, cyclic RGD peptide, IL4RPep-1, AS-1411 aptamer, anti-VEGF aptamer, A-9 aptamer, A-10 aptamer, anti-gp120 aptamer, TTA 1 aptamer, sgc8 aptamer, anti-MUC-1 aptamer, GBI-10 aptamer, anisamide, phenylboronic acid, folic acid, glucose, galactose, glutamate urea, vitamin A, mannose, and biotin. Such functional molecules include glycyrrhetinic acid, sulfonamide, and derivatives thereof.

Different functional molecules with similar or different functions can be used in combination to simultaneously impart multiple different functions and capabilities to the surface-engineered recombinant virus or viral genome. For example, two different cell-targeting ligands can be used together to increase the specificity of cell targeting. As another example, a cell-targeting ligand is used with an uptake-enhancing ligand to allow the surface-engineered recombinant virus or viral genome to both target a particular cell type and then to facilitate entry into the cell. In embodiments where different such molecules are used, each molecule is attached to a linker-spacer material such as silane-PEG as discussed above, and then the different molecule-PEG-silane conjugates are mixed together with the surface-engineered recombinant virus or viral genome to allow the different molecules to simultaneously attach to the surface-engineered recombinant virus or viral genome in the desired stoichiometry. The molecules and their associated functions are thus easily interchangeable during manufacturing.

As set forth herein, the surface can be further functionalized with stabilization and targeting ligands. In one particular embodiment, polyethylene glycol (PEG) is used to functionalize a silane group at one end and folate in the other hand. In particular embodiments, PEG can be a monomer, or PEG lengths can be selected from 1, 2, 3, 4, 5, 6, 7, 8, 9, 10, 11, 12, 13, 14, 15, 16, 17, 18, 19, or 20 KDa. PEG silane is added into the solution, and the silane group is hydrolyzed and attaches to the silica surface. pH-responsive linkers known to those of skill in the art can be used such that the PEG is stripped from the surface-engineered delivery system under conditions with altered pH, such as in the vicinity of tumors.

In certain embodiments, the PEG polymer is dual functionalized. On one end, it can be functionalized with silane and on the other end it can be functionalized with a targeting ligand like folate. Silane is used to attached the polymer to the ONCoat surface while folate can be utilized to target to the tissues and trigger cellular uptake, while PEG is being utilized to prevent neutralization and improve the pharmacokinetics. Targeting ligands include carbohydrates (e.g. galactose), monoclonal antibodies (e.g., anti-Her2, anti-EGFR), peptides (e.g., Arg-Gly-Asp or RGD), proteins (e.g., lectins, transferrin), vitamins (e.g., Vitamin D), and aptamers (e.g., RNA aptamers against HIV glycoprotein) and agonists/antagonists for toll-like receptors (TLR) such as TLR1, TLR2, TLR4, TLR7, TLR8, and TLR9 the like.

In certain other embodiments of the invention provided herein, the coating-layer further comprises molecules (e.g., binding agents such as targeting-ligands) on its surface that change the cellular uptake and/or biological activity of surface-engineered delivery system, for example the uptake or activity of the recombinant virus relative to the native-virus or the uptake or activity of the nucleic acid relative to the naked nucleic acid. Thus, the infectivity and host range of the surface-engineered or re-engineered virus can be selected and/or controlled by selecting and incorporating the appropriate targeting-ligands on the outer surface of the OnCoat artificial coating layer. The infectivity can thereby be increased. Additionally, this can reduce the dose required to obtain efficacy, and allow efficacy to be obtained at lower doses. When the virus is a vaccine, OnCoat increases the humoral immunity (antibody levels) and cellular immunity (T lymphocyte levels) produced by the vaccine. When the virus is an anti-cancer virus, OnCoat increases the anti-tumor efficacy. In particular embodiments of the surface-engineered delivery system, the artificial coating comprises a cell-surface ligand selected from the group consisting of: proteins, polysaccharides, aptamers, peptides, oligonucleotides and small molecules. In some embodiments, the artificial coating comprises a cell-penetrating molecule, such as a cell-penetrating peptide such as TAT, CADY, TP, or TP10. In some embodiments, the artificial coating comprises a targeting-ligand selected from the group consisting of: Antibodies, transferrin, Hyaluronic acid, RGD (e.g., cRGD), IL4RPep-1, AS-1411, GBI-10, Folate, anisamide, and phenylboronic acid. In particular embodiments, folate is used in the coating layer as ligand for binding to cells with folate receptors, such as cancer cells, and the like. In particular embodiments, antibodies against CD47 are used in the coating layer as ligand for binding to cells with surface expression of CD47, such as cancer cells, and the like. In some embodiments, the targeting-ligand is one that enables the surface-engineered delivery system to cross the blood-brain barrier, for example mannose or other glucose transporter ligands or binding molecules. Multiple different targeting-ligands can be combined to provide increased targeting specificity. Multiple different types of molecules in the coating-layer can be combined to provide multifunctionality, for example combining a targeting-ligand with a cell-penetrating molecule to enable the surface-engineered delivery system to both target and then enter a particular cell type.

In other embodiments, of the invention surface-engineered virus, the recombinant virus can be replication-competent or replication-defective, or “gutted” such that the virus does not encode any viral proteins. As used herein, the phrase “replication-defective virus” refers to a virus that is specifically defective for viral functions that are essential for viral genome replication and assembly of progeny virus particles. They are propagated in complementing cell lines that express the missing viral gene product(s), allowing viral replication to produce a stock of replication-defective virus.

Also provided herein is a surface-engineered recombinant virus vaccine, said virus comprising:

a recombinant Ad5 virus having a recombinant genome encoding SARS-CoV-2 spike protein, wherein at least a functional portion of E1 and E3 genes are deleted; and an artificial coating layer encapsulating the recombinant Ad5 virus. In a particular embodiment, as set forth herein, the artificial coating layer comprises an effective amount of ligand to bind a ligand receptor on a cell. In another embodiment, the artificial coating is selected from the group consisting of: silica, titanium oxide and calcium phosphate.

As used herein, the phrase “oncolytic virus” refers to a virus that preferentially infects and kills cancer cells. As the infected cancer cells are destroyed by oncolysis, they release new infectious virus particles or virions to help destroy the remaining tumor or tumors.

As used herein, the phrase “SARS-Cov-2 spike protein” refers to the glycoprotein on SARS-CoV-2 that promotes entry into cells using the ACE2 cell-surface receptor to enter cells. See, e.g., Walls et al., 2020, Cell, 180, 281-292 (which is incorporated herein by reference in its entirety for all purposes) for the spike protein sequence. Either the full-length spike protein sequence or any fragment thereof is contemplated for use herein with the invention artificially coated viral vaccines.

As used herein, the phrase “Ad5 virus” refers to the adenovirus serotype 5 that have been repeatedly used in humans to induce robust T cell-mediated immune (CMI) responses, all while maintaining an extensive safety profile (see, e.g., U.S. Pat. No. 9,605,276, and the like). Ad5 vectors can be reliably manufactured in large quantities and are stable for storage and delivery for outpatient administration.

In certain embodiments, the adenovirus vectors contemplated for use in the present invention include E1 and E3 deleted adenovirus vectors that have a deletion in the E1 and E3 region of the Ad genome and, optionally, the E2b region. In some cases, such vectors do not have any other regions of the Ad genome deleted. In another embodiment, the adenovirus vectors contemplated for use in the present invention include E1 deleted adenovirus vectors that have a deletion in the E1 region of the Ad genome and, optionally, deletions in the E3, E4 and/or E2b regions. In some cases, such vectors have no other regions deleted. In another embodiment, the adenovirus vectors contemplated for use in the present invention include E3 deleted adenovirus vectors that have a deletion in the E3 region of the Ad genome and, optionally, deletions in the E1, E4 and/or E2b regions. In some cases, such vectors have no other regions deleted. In a further embodiment, the adenovirus vectors contemplated for use in the present invention include adenovirus vectors that have a deletion in the E1 and E3 regions of the Ad genome and, optionally, deletions in the E2b and/or partial or complete removal of the E4 regions. In some cases, such vectors have no other deletions. In an additional embodiment, the adenovirus vectors contemplated for use in the present invention include adenovirus vectors that have a deletion in the E2a, E2b and/or E4 regions of the Ad genome. In some cases, such vectors have no other deletions. In one embodiment, the adenovirus vectors for use herein comprise vectors having the E1 and/or DNA polymerase functions of the E2b region deleted. In some cases, such vectors have no other deletions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase and/or the preterminal protein functions deleted. In some cases, such vectors have no other deletions. In one particular embodiment, the adenovirus vectors contemplated for use herein have at least a portion of the E2b region and/or the E1 region deleted. In some cases, such vectors are not “gutted” adenovirus vectors. In this regard, the vectors may have both the DNA polymerase and the preterminal protein functions of the E2b region deleted. In an additional embodiment, the adenovirus vectors for use in the present invention include adenovirus vectors that have a deletion in the E1, E2b and/or 100K regions of the adenovirus genome. In one embodiment, the adenovirus vectors for use herein comprise vectors having the E1, E2b and/or protease functions deleted. In some cases, such vectors have no other deletions. In a further embodiment, the adenovirus vectors for use herein have the E1 and/or the E2b regions deleted, while the fiber genes have been modified by mutation or other alterations (for example to alter Ad tropism). Removal of genes from the E3 or E4 regions may be added to any of the mentioned adenovirus vectors. In certain embodiments, the adenovirus vector may be a “gutted” adenovirus vector.

In certain embodiments, the virus can express additional cytokines (e.g., at least one subunit of IL12, GM-CSF and the like) or/and chemokines together with the at least one subunit of Spike protein to enhance improve the tumor engagement.

As used herein, the phrase “E1 deleted” or “a functional portion of the E1 gene is deleted,” or grammatical variations thereof, refers to a specific DNA sequence that is mutated in such a way so as to prevent expression and/or function of at least one E1 gene product. Thus, in certain embodiments, “E1 deleted” is used in relation to a specific DNA sequence that is deleted (removed) from the Ad genome. E1 deleted or “containing a deletion within the E1 region” refers to a deletion of at least one base pair within the E1 region of the Ad genome. Thus, in certain embodiments, more than one base pair is deleted and in further embodiments, at least 20, 30, 40, 60, 70, 80, 90, 100, 110, 120, 130, 140, or 150 base pairs are deleted. In another embodiment, the deletion is of more than 150, 160, 170, 180, 190, 200, 250, or 300 base pairs within the E1 region of the Ad genome. An E1 deletion may be a deletion that prevents expression and/or function of at least one E1 gene product and therefore, encompasses deletions within exons of encoding portions of E1-specific proteins as well as deletions within promoter and leader sequences. In certain embodiments, an E1 deletion is a deletion that prevents expression and/or function of one or both of a trans-acting transcriptional regulatory factor of the E1 region. In a further embodiment, “E1 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As used herein, the phrase “E3 deleted” or “a functional portion of the E3 gene is deleted,” or grammatical variations thereof, refers to a specific DNA sequence that is mutated in such a way so as to prevent expression and/or function of at least one E3 gene product. Thus, in certain embodiments, “E3 deleted” is used in relation to a specific DNA sequence that is deleted (removed) from the Ad genome. E3 deleted or “containing a deletion within the E3 region” refers to a deletion of at least one base pair within the E3 region of the Ad genome. Thus, in certain embodiments, more than one base pair is deleted and in further embodiments, at least 20, 30, 40, 60, 70, 80, 90, 100, 110, 120, 130, 140, or 150 base pairs are deleted. In another embodiment, the deletion is of more than 150, 160, 170, 180, 190, 200, 250, or 300 base pairs within the E3 region of the Ad genome. An E3 deletion may be a deletion that prevents expression and/or function of at least one E3 gene product within the expression cassette and therefore, encompasses deletions within exons of encoding portions of E3-specific proteins as well as deletions within promoter and leader sequences. In certain embodiments, an E3 deletion is a deletion that prevents expression and/or function of at least one of the host immune response modulating proteins of the E3 region (see, e.g., Arnberg, PNAS, 2013, 110(50):19976-19977). In a further embodiment, “E3 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As used herein, the phrase “E2b deleted” or “a functional portion of the E2b gene is deleted,” or grammatical variations thereof, refers to a specific DNA sequence that is mutated in such a way so as to prevent expression and/or function of at least one E2b gene product. Thus, in certain embodiments, “E2b deleted” is used in relation to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein. Deletions or mutations in the Ad genome can be within one or more of E1a, E1b, E2a, E2b, E3, E4, L1, L2, L3, L4, L5, TP, POL, IV, and VA regions.

The deleted adenovirus vectors of the present invention can be generated using recombinant techniques known in the art (see e.g., Amalfitano et al., 1998 J. Virol. 72:926-933; Hodges, et al., 2000 J Gene Med 2:250-259).

Also provided herein is a method of making a surface-engineered recombinant virus, said method comprising:
producing a recombinant virus having a recombinant genome, or a replication-competent oncolytic virus set forth herein; and
applying an artificial coating layer to the recombinant virus, wherein the artificial coating is selected from the group consisting of: silica, titanium oxide and calcium phosphate.

As set forth herein, any of the viruses described herein can be genetically modified using method well-known in the art to produce a recombinant virus having a recombinant genome. The artificial coating layer can be applied to the recombinant virus as described herein.

Also provided herein, is a method of re-engineering the surface of a virus having a native-envelope, said method comprising:
removing the native-envelope from the virus, or a replication-competent oncolytic virus set forth herein, to isolate a previously-enveloped-capsid; and applying an artificial coating to the previously-enveloped-capsid.

Any enveloped virus or lipid-containing virus known in the art is suitable for re-engineering using the methods provided herein.

As used herein, the phrase “re-engineering the surface of a virus having a native-envelope” refers to proactively disrupting and replacing the viral envelope (e.g., outer lipid-containing layer) of an enveloped-virus with an artificial coating layer.

As used herein, the phrase “removing the native-envelope from the virus” refers to using any means to disrupt the native outer lipid-containing layer enveloping the capsid, such that the capsid, referred to herein as the previously-enveloped-capsid, can be isolated from any remaining lipids or outer envelope layer.

As used herein, the phrase “previously-enveloped-capsid” refers to an envelope-free or naked-capsid isolated from a previously enveloped virus, such as those described herein.

The envelope of the virus can be removed using any method known in the art, including use of detergents and/or delipidation with an extraction solvent as set forth in U.S. Pat. No. 7,407,662 (which in incorporated herein in its entirety for all purposes). In particular embodiments, the native-envelope is removed or delipidated using a detergent and/or an extraction solvent. In some embodiments, the detergent and/or an extraction solvent can be selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination.

As used herein, the term “delipidation” refers to the process of removing at least a portion of a total concentration of lipids in a fluid or in a lipid-containing envelope of an enveloped virus described herein.

Briefly, in one embodiment, the envelope is removed and the artificial coating layer is applied as follows. The recombinant virus is incubated in aqueous buffer with 0.5-1% NP-40 (detergent) for 5 mins at room temperature. Then, the aqueous buffer is washed three times with detergent free aqueous buffer to remove the detergent and lifted envelope.

In other embodiments of the methods provided herein, a first extraction solvent is used to remove the envelope from an enveloped virus, to produce a fluid solution containing a previously-enveloped-capsid. As used herein the phrase, “first solvent” or “first organic solvent” “or first extraction solvent,” or grammatical variations thereof, refers to a solvent, comprising one or more solvents, used to facilitate extraction of lipid envelope from a lipid-containing envelope virus in the fluid. This solvent will enter the fluid and remain in the fluid until being removed. Suitable first extraction solvents include solvents that extract or dissolve lipid, including but not limited to alcohols, hydrocarbons, amines, ethers, and combinations thereof. First extraction solvents may be combinations of alcohols and ethers. First extraction solvents include, but are not limited to n-butanol, di-isopropyl ether (DIPE), diethyl ether, and combinations thereof.

In particular embodiments, second extraction solvent is used to remove the first solvent from the solution containing the de-enveloped capsid (“previously-enveloped-capsid”). As used herein the phrase “second extraction solvent” reefers to one or more solvents that may be employed to facilitate the removal of a portion of the first extraction solvent. Suitable second extraction solvents include any solvent that facilitates removal of the first extraction solvent from the fluid.

Second extraction solvents include any solvent that facilitates removal of the first extraction solvent including but not limited to ethers, alcohols, hydrocarbons, amines, and combinations thereof.

Preferred second extraction solvents include diethyl ether and di-isopropyl ether, which facilitate the removal of alcohols, such as n-butanol, from the fluid. The term “de-emulsifying agent” is a second extraction solvent that assists in the removal of the first solvent which may be present in an emulsion in an aqueous layer.

In another embodiment, the envelope on either a naturally occurring or recombinant enveloped virus is removed and replaced or artificially coated with an OnCoat coating (e.g., silica-gel coating, and the like) as described herein. See, for example, the methods described in US 2019/0151253A1 and in Ortac et al. (Nano Lett. 2014, 14, 6, 3023-3032), which are incorporated herein by reference in their entirety, for all purposes. Once the envelope is removed, then the remaining capsid can be coated with an OnCoat artificial coating layer as described herein.

As set forth herein, in certain embodiments, the artificial coating is selected from the group consisting of: silica, titanium oxide and calcium phosphate. In one embodiment, applying the artificial coating further comprises conducting a charge-mediated sol-gel condensation reaction directly onto the surface of the previously-enveloped-capsid. In another embodiment, the artificial coating comprises a silica gel matrix or titanium oxide gel matrix encapsulating the previously-enveloped-capsid. In particular embodiments, the previously-enveloped-capsid is replication-competent or replication-defective.

In particular embodiments for applying the artificial coating layer, as set forth in the methods described in US 2019/0151253A1, which is incorporated herein by reference in its entirety for all purposes, the virus is incubated with polycationic polymer to shift the virus surface charge towards the positive direction. Next, a silica precursor, such as tetramethyl (or tetraethyl) orthosilicate, and the like, is hydrolyzed to generate silicic acid, which is added into the virus suspension surface-modified with polycationic polymer. The polymer on the virus surface templates the polycondensation reaction (sol-gel) to produce silica gel, as set forth in US which is incorporated herein by reference in its entirety for all purposes.

The surface can then be further functionalized with stabilization and targeting ligands. In one particular embodiment, polyethylene glycol (PEG) is used to functionalize a silane group at one end and folate in the other hand. In particular embodiments, PEG can be a monomer, or PEG lengths can be selected from 1, 2, 3, 4, 5, 6, 7, 8, 9, 10, 11, 12, 13, 14, 15, 16, 17, 18, 19, or KDa. The PEG can be a single PEG monomer or polymer, or it can be multiple PEG monomers or polymers, for example a mixture of different PEG polymers with different molecular weights. PEG silane is added into the solution, and the silane group is hydrolyzed and attaches to the silica surface.

The infectivity and host range of the surface-engineered or re-engineered virus can be selected and/or controlled by selecting and incorporating the appropriate targeting-ligands on the outer surface of the OnCoat artificial coating layer. The infectivity can thereby be increased. Additionally, this can reduce the dose required to obtain efficacy, and allow efficacy to be obtained at lower doses. When the virus is a vaccine, OnCoat increases the humoral immunity (antibody levels) and cellular immunity (T lymphocyte levels) produced by the vaccine. When the virus is an anti-cancer virus, OnCoat increases the anti-tumor efficacy.

Any naturally occurring enveloped virus can be de-enveloped and coated with the OnCoat outer layer. Exemplary enveloped viruses or lipid containing viruses contemplated for use herein include: Poxvirus (e.g., vaccinia virus, and the like), Herpes Virus (e.g., herpes simplex, and the like), Rhabdovirus (e.g., Vesicular stomatitis virus, and the like), Coronavirus (e.g., SARS-CoV-2, and the like), Hepadnaviruses, Asfarviridae, Flavivirus, Alphavirus, Togavirus, Hepatitis D, Orthomyxovirus, Paramyxovirus, Bunyavirus, Filovirus, Retroviruses, Alphavirus (alphaviruses), Rubivurus (rubella virus), Flavivirus (Flaviviruses), Pestivirus (mucosal disease viruses), hepatitis C virus, Coronavirus, (Coronaviruses), severe acute respiratory syndrome (SARS), Torovirus, (toroviruses), Arteivirus, (arteriviruses), Paramyxovirus, (Paramyxoviruses), Rubulavirus (rubulavriuses), Morbillivirus (morbillivuruses), Pneumovirinae (the pneumoviruses), Pneumovirus (pneumoviruses), Vesiculovirus (vesiculoviruses), Lyssavirus (lyssaviruses), Ephemerovirus (ephemeroviruses), Cytorhabdovirus (plant rhabdovirus group A), Nucleorhabdovirus (plant rhabdovirus group B), Filovirus (filoviruses), Influenzavirus A, B (influenza A and B viruses), influenza virus C (influenza C virus), (unnamed, Thogoto-like viruses), Bunyavirus (bunyaviruses), Phlebovirus (phleboviruses), Nairovirus (nairoviruses), Hantavirus (hantaviruses), Tospovirus (tospoviruses), Arenavirus (arenaviruses), unnamed mammalian type B retroviruses, unnamed, mammalian and reptilian type C retroviruses, unnamed, type D retroviruses, Lentivirus (lentiviruses), Spumavirus (spumaviruses), Orthohepadnavirus (hepadnaviruses of mammals), Avihepadnavirus (hepadnaviruses of birds), Simplexvirus (simplexviruses), Varicellovirus (varicelloviruses), Betaherpesvirinae (the cytomegaloviruses), Cytomegalovirus (cytomegaloviruses), Muromegalovirus (murine cytomegaloviruses), Roseolovirus (human herpes virus 6, 7, 8), Gammaherpesvirinae (the lymphocyte-associated herpes viruses), Lymphocryptovirus (Epstein-Barr-like viruses), Rhadinovirus (saimiri-ateles-like herpes viruses), Orthopoxvirus (orthopoxviruses), Parapoxvirus (parapoxviruses), Avipoxvirus (fowlpox viruses), Capripoxvirus (sheeppox-like viruses), Leporipoxvirus (myxomaviruses), Suipoxvirus (swine-pox viruses), Molluscipoxvirus (molluscum contagiosum viruses), Yatapoxvirus (yabapox and tanapox viruses), Unnamed, African swine fever-like viruses, Iridovirus (small iridescent insect viruses), Ranavirus (front iridoviruses), Lymphocystivirus (lymphocystis viruses of fish), Togaviridae, Flaviviridae, Coronaviridae, Enabdoviridae, Filoviridae, Paramyxoviridae, Orthomyxoviridae, Bunyaviridae, Arenaviridae, Retroviridae, Hepadnaviridae, Herpesviridae, Poxviridae, and any other lipid-containing, enveloped virus.

In other embodiments, the virus can be selected from the following human and animal pathogens: Ross River virus, fever virus, dengue viruses, Murray Valley encephalitis virus, tick-borne encephalitis viruses (including European and far eastern tick-borne encephalitis viruses, California encephalitis virus, St. Louis encephalitis virus, sand fly fever virus, human coronaviruses 229-E and OC43 and others causing the common cold, upper respiratory tract infection, probably pneumonia and possibly gastroenteritis), human parainfluenza viruses 1 and 3, mumps virus, human parainfluenza viruses 2, 4a and 4b, measles virus, human respiratory syncytial virus, rabies virus, Marburg virus, Ebola virus, influenza A viruses and influenza B viruses, Arenavirus: lymphocytic choriomeningitis (LCM) virus; Lassa virus, human immunodeficiency viruses 1 and 2, or any other immunodeficiency virus, hepatitis B virus, hepatitis C virus, hepatitis G virus, Subfamily: human herpes viruses 1 and 2, herpes virus B, Epstein-Barr virus), (smallpox) virus, cowpox virus, monkeypox virus, molluscum contagiosum virus, yellow fever virus, poliovirus, Norwalk virus, orf virus, and any other lipid-containing, enveloped virus. See, e.g., Mathews, J. gen. Virol. (1975), 27:135-149; which is incorporated herein by reference in its entirety for all purposes.

Also provided herein is a surface-re-engineered virus comprising:

a previously-enveloped-capsid from a naturally occurring enveloped-virus, or a replication-competent oncolytic virus set forth herein; and an artificial coating layer surrounding the previously-enveloped-capsid.

As used herein, “re-engineered virus” refers to an artificial transducing agent that has a capsid from a previously enveloped virus, wherein the previously-enveloped-capsid is completely coated or completely encapsulated by an artificial coating layer (i.e., artificial coating).

As set forth herein, the artificial coating (also referred to herein as “OnCoat”) is selected from the group consisting of: silica, titanium oxide and calcium phosphate. In certain embodiments, the artificial coating is applied by conducting a charge-mediated sol-gel condensation reaction directly onto the surface of the previously-enveloped-capsid. In other embodiments, the artificial coating comprises a silica gel matrix or titanium oxide gel matrix encapsulating the previously-enveloped-capsid. The previously-enveloped-capsid can be either replication-competent or replication-defective.

Also provided herein is a method of re-engineering a virus having a native-envelope, said method comprising:
removing the native-envelope surrounding a capsid from the virus, or a replication-competent oncolytic virus set forth herein; isolating the previously-enveloped-capsid; and
applying an artificial coating to the previously-enveloped-capsid to form an re-engineered virus.

In other embodiments, the recombinant virus genome or nucleic acid can encode molecular components used for genome editing, for example genome editing nucleases. Such genome editing nucleases can be Cas and related nucleases (e.g., Cas3, Cas9, CasX) for CRISPR, TALE nucleases, zinc finger nucleases, PPR nucleases, or other nucleases. Surface-engineered delivery systems of such embodiments can thus be used in methods of in vivo, in vitro, or ex vivo genome editing.

### Treatment Methods

In an embodiment of the present invention, a method of treatment for a hyperproliferative or neoplastic disease, such as cancer, by the delivery of the recombinant virus (including surface-engineered recombinant virus) or surface-engineered delivery system, is contemplated. Examples of cancer contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions, pre-neoplastic lesions in the lung, colon cancer, melanoma, bladder cancer and any other cancers or tumors that may be treated, including metastatic or systemically distributed cancers, as well as those cancers disclosed elsewhere in this application. A “patient” or “subject” for the purposes of the present invention includes both humans and other animals, particularly mammals. Thus, the pharmaceutical preparations for use according to the invention are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine). Thus the methods are applicable to both human therapy and veterinary applications. In the preferred embodiment the patient is a mammal, preferably a primate, and in the most preferred embodiment the patient is human.

An effective amount of the pharmaceutical composition, generally, is defined as that amount sufficient to detectably and repeatedly to slow, ameliorate, reduce, minimize, or limit the extent of the disease or its symptoms. More rigorous definitions may apply, including elimination, eradication, or cure of disease.

Preferably, patients will have adequate bone marrow function (defined as a peripheral absolute granulocyte count of >2,000/mm3 and a platelet count of 100,000/mm3), adequate liver function (bilirubin<1.5 mg/dl) and adequate renal function (creatinine<1.5 mg/dl).

### Administration

To kill cells, inhibit cell growth, inhibit metastasis, decrease tumor or tissue size, and otherwise reverse, stay, or reduce the malignant phenotype of tumor cells, using the methods and compositions of the present invention, one would generally contact a hyperproliferative or neoplastic cell with a therapeutic composition such as a recombinant virus (including surface-engineered recombinant virus) or an expression construct encoding a polypeptide, where the expression construct is delivered by the surface-engineered delivery system. The routes of administration will vary, naturally, with the location and nature of the lesion, and include, e.g., intradermal, transdermal, parenteral, intravascular, intravenous, intramuscular, intranasal, subcutaneous, regional, percutaneous, intratracheal, intraperitoneal, intraarterial, intravesical, intratumoral, inhalation, perfusion, lavage, direct injection, alimentary, and oral administration and formulation.

To effect a therapeutic benefit with respect to a vascular condition or disease, one would contact a vascular cell with the therapeutic compound. Any of the formulations and routes of administration discussed with respect to the treatment or diagnosis of cancer may also be employed with respect to vascular diseases and conditions.

Intratumoral injection, or injection into the tumor vasculature is contemplated for discrete, solid, accessible tumors. Local, regional or systemic administration is also contemplated, particularly for those cancers that are disseminated or are likely to disseminated systemically. The recombinant virus (including surface-engineered recombinant virus) or surface-engineered delivery system may be administered by at least or at most 1, 2, 3, 4, 5, 6, 7, 8, 9, 10 injections.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a recombinant virus (including surface-engineered recombinant virus), which may or may not harbor one or more mutations or one or more transgenes, or surface-engineered delivery system of the present invention that is advantageous for treatment of cancer or cancer cells. The perfusion may be continued post-resection, for example, by leaving a catheter implanted at the site of the surgery. Periodic post-surgical treatment also is envisioned.

The compositions of the present invention may be delivered as a bolus or by continuous infusion over a period of time. The administration may be local or systemic. Continuous administration may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 1, 2, 3, 4, 5, 6 or more dose application over a 1, 2, 3, 4, 5, 6-week period or more. A two-week regimen may be repeated one, two, three, four, five, six or more times. During a course of treatment, the need to complete the planned dosings may be re-evaluated.

The treatments may include various “unit doses.” Unit dose is defined as containing a predetermined quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for viral constructs. Unit doses range from 103, 104, 105, 106, 107, 108, 109, 1010, 1011, 1012, 1013 pfu or vp and higher. Alternatively, depending on the kind of virus and the titer attainable, one will deliver 1 to 100, 10 to 50, 100-1000, or up to about 1×104, 1×105, 1×106, 1×107, 1×108, 1×109, 1×1010, 1×1011, 1×1012, 1×1013, 1×1014, or 1×1015 or higher infectious viral particles (vp) to the patient or to the patient's cells. Dosages can be empirically determined considering the type and stage of cancer diagnosed in a particular patient. The dose administered to a patient, in the context of the present invention should be sufficient to affect a beneficial therapeutic response in the patient over time. The size of the dose also will be determined by the existence, nature, and extent of any adverse side-effects that accompany the administration of a particular composition of the invention in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

### Injectable Compositions and Formulations

The preferred method for the delivery of the recombinant virus (including surface-engineered recombinant virus) or surface-engineered delivery system of the present invention to cancer or tumor cells in the present invention is via intravascular injection. However, the pharmaceutical compositions disclosed herein may alternatively be administered intratumorally, parenterally, intravenously, intrarterially, intradermally, intramuscularly, transdermally or even intraperitoneally as described in U.S. Pat. Nos. 5,543,158, 5,641,515 and 5,399,363 (each specifically incorporated herein by reference in its entirety).

Injection of the compositions of the present invention may be delivered by syringe or any other method used for injection of a solution, as long as the composition can pass through the particular gauge of needle required for injection (for examples see U.S. Pat. Nos. 5,846,233 and 5,846,225).

Pharmaceutical compositions of the recombinant virus (including surface-engineered recombinant virus) or surface-engineered delivery system may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

For parenteral administration in an aqueous solution or dispersion, for example, the solution or dispersion should be suitably buffered if necessary and the liquid diluent first rendered isotonic with sufficient saline or glucose. These particular aqueous solutions and dispersions are especially suitable for intravenous, intramuscular, subcutaneous, intratumoral, and intraperitoneal administration. In this connection, sterile aqueous media that can be employed will be known to those of skill in the art in light of the present disclosure. For example, one dosage may be dissolved, dispersed, or suspended in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards required by governments of the countries in which the compositions are being used.

The recombinant virus or surface-engineered delivery system of the invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. Pharmaceutical formulations, particularly, of the recombinant viruses and surface-engineered delivery systems of the present invention can be prepared by mixing the recombinant virus or surface-engineered delivery system having the desired degree of purity with optional pharmaceutically acceptable carriers, excipients or stabilizers. Such formulations can be lyophilized formulations or aqueous solutions. Acceptable carriers, excipients, or stabilizers are nontoxic to recipients at the dosages and concentrations used. Acceptable carriers, excipients or stabilizers can be acetate, phosphate, citrate, and other organic acids; antioxidants (e.g., ascorbic acid) preservatives low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g. Zn-protein complexes); and/or non-ionic surfactants. The recombinant virus or surface-engineered delivery system can be formulated at any appropriate concentration.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

### Example 1: Generation of Oncolytic Viruses

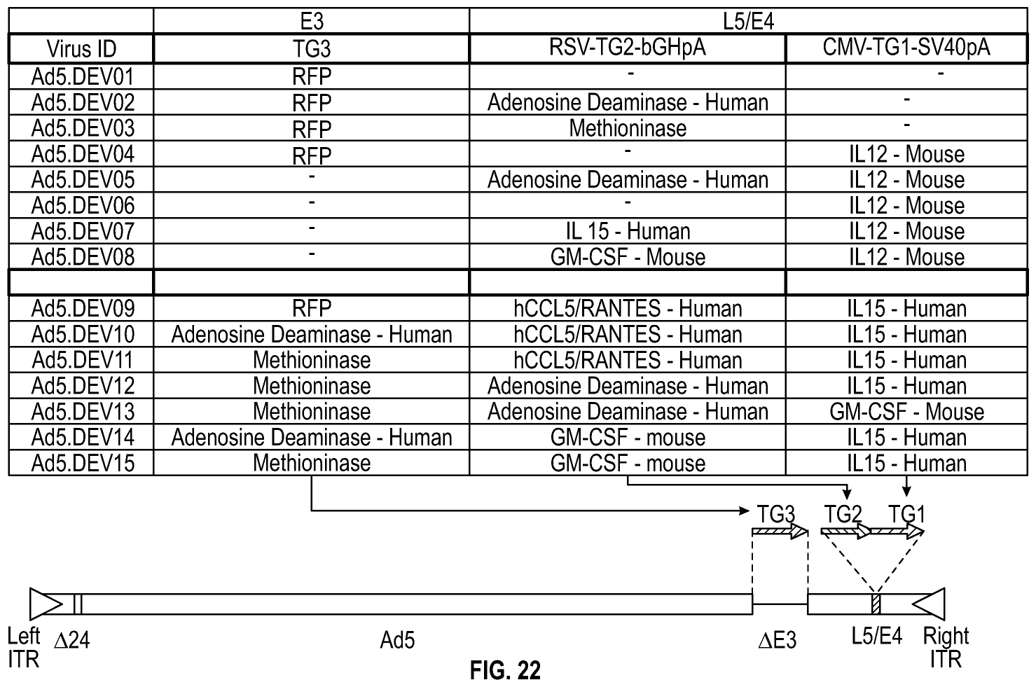

Ad5-based oncolytic viruses were generated by inserting one, two, or three transgenes into the Ad5 backbone. The resulting Ad5 oncolytic viruses have an Ad5 backbone (human adenovirus C serotype 5; Gb AY339865) with a 24-bp deletion in E1A Conserved Region 2 (CR2) and a 2428-bp BsiWI-BglII deletion in the E3 region into which the third transgene is inserted. Transgenes inserted in this location should be expressed in the early phase of virus replication. Expression cassettes including the first and second transgenes under control of CMV and RSV promoters, respectively, were inserted in tandem between the L5 and E4 poly-A signals. FIG. 22 shows a tabular representation of the transgenes and the respective promoter driving each transgene, as well as a schematic representation of the Ad5 viral backbone showing where each transgene was inserted and also showing the 24-bp deletion in E1A Conserved Region 2.

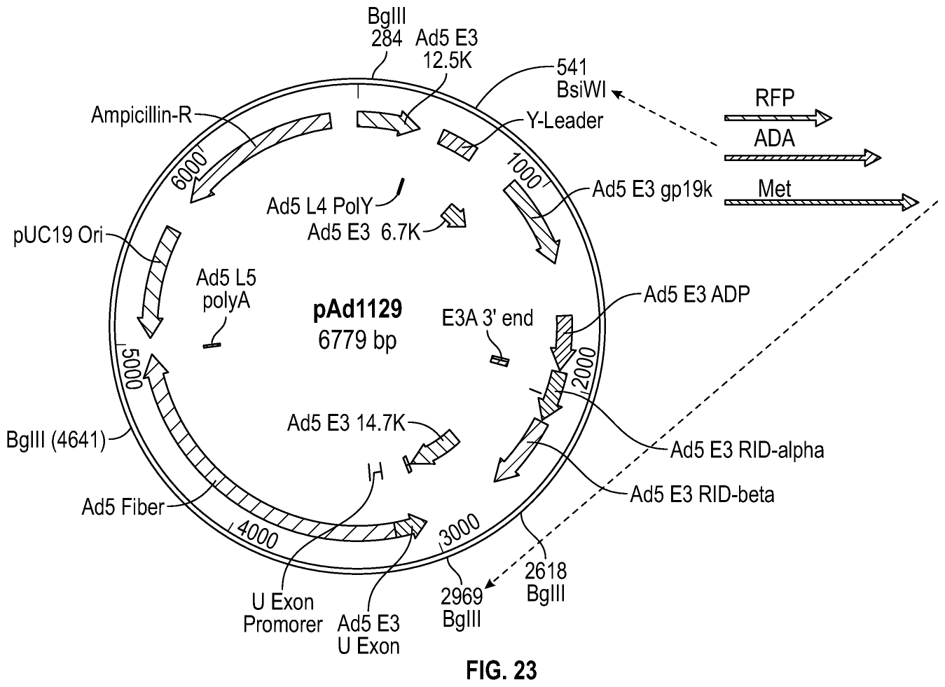

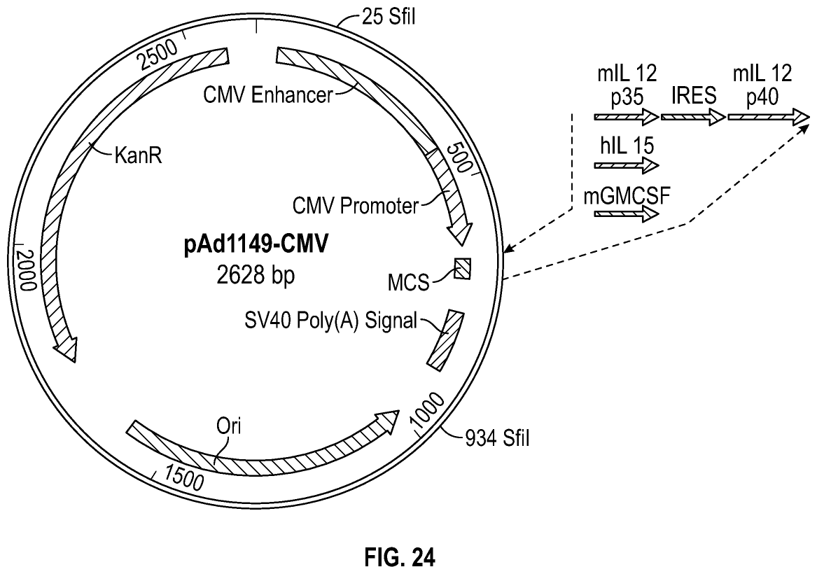

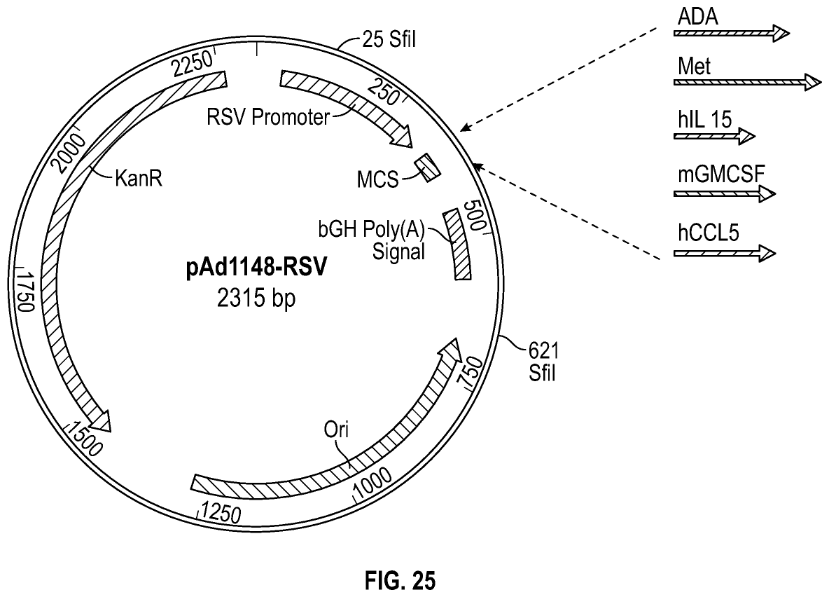

The transgenes were inserted into the Ad5 backbone using shuttle plasmids. The third transgene encodes either: human adenosine deaminase (SEQ ID NOS:3 & 4: human, accession #BC040226), Pseudomonas putida L-methionine gamma-lyase (methioninase), or red fluorescent protein as a reporter gene, and was inserted into the pAd1129 plasmid between the BsiWI and BglII site located in the E3 region of pAd1129 to form shuttle plasmids pAd1129-RFP, pAd1129-ADA and pAd1129-Met. Methioninase is set forth in SEQ ID NOS: 5 & 6; and corresponds to the sequence from Tan et al. (Protein Expression and Purification, 9: 233-245), which is referenced by Miki et al. (Cancer Research, 60: 2696-2702). This sequence has one silent mutation compared to the published sequence with accession #D88554. A methioninase sequence after mouse-codon optimization corresponding to SEQ ID NOS:7 &8, is also contemplated for use herein. The shuttle plasmid design is shown in FIG. 23. Using the shuttle plasmid, the transgene was then inserted into a 2428-bp BsiWI-BglII deletion in the E3 region of the Ad5 backbone as described above. The first transgene encodes: mouse IL-12 (SEQ ID NOS:9 & 10; mIL12 p35 subunit: Mus musculus interleukin 12a (IL12a), transcript variant 2, accession #NM_008351.3; or SEQ ID NOS:11 & 12; mIL12 p40 subunit: Mus musculus interleukin 12b (IL12b), accession #NM_001303244.1), human IL-15 (SEQ ID NOS:13 & 14; human, accession #NM_172174 or NM_000585), or mouse GM-CSF (SEQ ID NOS:15 & 16; mouse, accession #NM_009969) and was inserted into the multiple cloning site of the pAd1149-CMV plasmid. For IL-12, a dicistronic cassette mIL12p35-IRES-mIL12p40 expressing the p35 (IL12-a) and p40 (IL-12b) subunits of IL-12 was used. The shuttle plasmid design is shown in FIG. 24. Using the shuttle plasmid, the transgene was then inserted into between the L5 and E4 poly-A signals in the Ad5 backbone as described above. The second transgene encodes human CCL5 (SEQ ID NOS: 17 & 18; human, accession #NM_002985.3), human adenosine deaminase (SEQ ID NOS:3 & 4: human, accession #BC040226), or mouse GM-CSF (SEQ ID NOS:15 & 16) and was inserted into the multiple cloning site of the pAd1148-RSV plasmid. The shuttle plasmid design is shown in FIG. 25. Using the shuttle plasmid, the transgene was then inserted between the L5 and E4 poly-A signals in the Ad5 backbone as described above.

To produce the viruses, cosmids containing the entire genomes of the recombinant viruses were constructed by combining the plasmids as described above. The particular shuttle plasmids used are as follows:

TABLE 2

| | E1/pIX | E2/late | E3/Fiber | | | E4 |
|---|---|---|---|---|---|---|
| | shuttle | shuttle | shuttle | L5/E4 shuttles | | shuttle |
| pAd5- | pAd1127-18 | pAd1128 | pAd1129- | pAd1148 | pAd1149 | pAd1130 |
| DEV01 | | | RFP | | | |
| pAd5- | pAd1127-18 | pAd1128 | pAd1129- | pAd1148- | pAd1149 | pAd1130 |
| DEV02 | | | RFP | ADA | | |
| pAd5- | pAd1127-18 | pAd1128 | pAd1129- | pAd1148- | pAd1149 | pAd1130 |
| DEV03 | | | RFP | Met | | |
| pAd5- | pAd1127-18 | pAd1128 | pAd1129- | pAd1148 | pAd1149- | pAd1130 |
| DEV04 | | | RFP | | mIL12 | |
| pAd5- | pAd1127-18 | pAd1128 | pAd1129- | pAd1148- | pAd1149- | pAd1130 |
| DEV05 | | | 53 | ADA | mIL12 | |
| pAd5- | pAd1127-18 | pAd1128 | pAd1129- | pAd1148 | pAd1149- | pAd1130 |
| DEV06 | | | 53 | | mIL12 | |
| pAd5- | pAd1127-18 | pAd1128 | pAd1129- | pAd1148- | pAd1149- | pAd1130 |
| DEV07 | | | 53 | hIL15 | mIL12 | |
| pAd5- | pAd1127-18 | pAd1128 | pAd1129- | pAd1148- | pAd1149- | pAd1130 |
| DEV08 | | | 53 | mGMCSF | mIL12 | |
| pAd5- | pAd1127-18 | pAd1128 | pAd1129- | pAd1148- | pAd1149- | pAd1130 |
| DEV09 | | | RFP | hCCL5 | hIL15 | |
| pAd5- | pAd1127-18 | pAd1128 | pAd1129- | pAd1148- | pAd1149- | pAd1130 |
| DEV10 | | | ADA | hCCL5 | hIL15 | |
| pAd5- | pAd1127-18 | pAd1128 | pAd1129- | pAd1148- | pAd1149- | pAd1130 |
| DEV11 | | | Met | hCCL5 | hIL15 | |
| pAd5- | pAd1127-18 | pAd1128 | pAd1129- | pAd1148- | pAd1149- | pAd1130 |
| DEV12 | | | Met | ADA | hIL15 | |
| pAd5- | pAd1127-18 | pAd1128 | pAd1129- | pAd1148- | pAd1149- | pAd1130 |
| DEV13 | | | Met | ADA | mGMCSF | |
| pAd5- | pAd1127-18 | pAd1128 | pAd1129- | pAd1148- | pAd1149- | pAd1130 |
| DEV14 | | | ADA | mGMCSF | hIL15 | |
| pAd5- | pAd1127-18 | pAd1128 | pAd1129- | pAd1148- | pAd1149- | pAd1130 |
| DEV15 | | | Met | mGMCSF | hIL15 | |

In the cosmids shown in TABLE 2, pAd1127-18 is a shuttle plasmid that contains the Ad5 E1 region with a 24-bp deletion in conserved region 2 (CR2). pAd1128 is a shuttle plasmid that contains the WT Ad5 E2 region and most of the late genes. pAd1129-53 is a shuttle plasmid that contains the Ad5 E3 and fiber genes, with a 2428-bp BsiWI-BglII deletion in the E3 region. pAd1148 and pAd1149 are shuttle plasmids designed for inserting tandem expression cassettes between the L5 and E4 polyA signal. pAd1130 is a shuttle plasmid that contains the WT E4 region. All plasmids were sourced from O.C. 260, Inc.

The adenovirus genomes were then excised from the cosmids and transfected into 293 and A549 cells. 293 cells are easy to transfect and very permissive for adenovirus growth, but give the possibility of generating revertant viruses by recombination between the virus genome and the Ad5 sequences embedded in the 293 cell chromosomes. A549 cells are more difficult to transfect and do not always support efficient virus growth, but do not generate revertants like the 293 cells. Plaques obtained from A549 cells were thus given priority. Plaques obtained from 293 cells served as backup in case of failure with A549 cells. For each construct, three virus plaques were harvested and amplified in small scale. Their identity was verified by restriction analysis of their genome (Hirt supernatant).

For production, viral clones selected as described above were then amplified and purified on two CsCl gradients and chromatography, and then dialyzed against an isotonic buffer (GTS buffer: 2.5% glycerol, 25 mM NaCl, 20 mM Tris-HCl, pH 8.0) and finally sterilely filtered through a low-protein-binding 0.22 μm filter. The virus particle concentration was determined by ultraviolet absorbance (Abs260-SDS method). The amplifications were based on the infection of 8 cells and yielded 10¹² viral particles. The presence of replication-competent adenovirus (RCA) in a virus stock was assessed using a modified infectivity/PCR method, which combines the amplification of infectious RCA by cell culture with the sensitivity of detection of E1-specific sequences by PCR (Ishii-Watabe et al., 2003, Mol. Ther. 8, 1009-1016).

### Example 2: Surface Engineering a Virus

### ONCoat Reaction Procedure

A cationic polymer is dissolved in a buffer solution, typically 1×PBS or similar. Final concentration is determined with respect to the diameter of the viral particles, their concentration and their surface charge. In a typical reaction for Ad5, poly-1-lysine (PLL) with 30-70 KDa molecular weight is dissolved in 1×PBS in a final concentration of around 0.05-0.1% weight percentage. Then, viral particles are added into the solution while it is being gently shaken/vortexed. Adding viral particles into a cationic polymer solution improves their solution stability. In a typical Ad5 reaction, the final concentration of viral particles in solution are around 0.5-1×10¹¹ particles/ml. The suspension is vortexed for about 1-5 minutes.

Subsequently, silicic acid solution is added to the solution. The volume ratio of silicic acid to the virus suspension solution is around 1/200. Silicic acid solution is prepared by mixing 500 parts of 1 mM HCl with 74 parts tetra-methyl orthosilicate (TMOS). This will initiate the polycondensation reaction. The solution is typically vortexed gently for 1 hour. This procedure applies an ONCoat surface-engineered layer around the virus surface.

### Surface Functionalization

For many applications, this process is continued with surface functionalization to improve stability in biological fluids, to improve tissue/cell specific targeting and cellular uptake. In particular, ligands with silane functional groups can be added to the surface efficiently with one step reaction.

For this embodiment of ONCoat, poly ethylene glycol (PEG) (e.g., 1, 5, 10, 20, 30 KDa) functionalized with silane at one end and folate at the other end (silane-PEG-folate) that provides good stability in blood and in the tissue and high efficiency cellular uptake mainly through folate receptors. This molecule was attached to ONCoat surface with a one-step reaction. 10 KDa PEG was found to be very optimal, thus silane-PEG10K-folate is the most commonly used configuration provided herein due to the good physical and biological characteristics.

Following the ONCoat surface-engineering procedure, while the ONCoat virus particles are still being gently shaken, silane-PEG10K-Folate suspended in the same buffer (i.e. 1×PBS) with a final concentration of 5 mg/ml (typically in the range of 1-50 mg/ml) is mixed with ONCoat Ad5 solution. This mixture is allowed to react for 1-2 hours at room temperature.

### Silane-PEG-Folate Functionalization

Most mammalian cells have folate receptors and can take up folate modified nanoparticles and biologics. It is over-expressed in cancer cells providing an extra selectivity in the tumor tissue. However, it is also useful for non-cancer applications such as vaccines. Typically, ONCoat itself provides a net negative charge. This makes the particle prone to opsonization in blood. PEG functionalization neutralizes the charge and stabilizes the particles in biological fluids. However, it reduces the infection efficiency in most cases. When each PEG molecule on the ONCoat surface is terminated by a folate group, the infection efficiency is improved significantly while the particles are still stable in biological fluids. The surface engineering can comprise having a mixture of folate molecules and PEG molecules, both directly attached to the surface. However, the folate groups among PEG groups might have limited access to the receptors on the cell surface due to blocking by the PEG. The inventors have thus found that for many applications the best configuration is functionalizing the surface with PEG molecules, where each PEG molecule has a folate group at the end. The inventors have also found that if the other end of the PEG polymer has silane, it provides a surprisingly efficient and dense functionalization of the ONCoat surface. Thus, silane-PEG10K-folate functionalization can be used for many applications from oncolytic virotherapy, to gene therapy to vaccines. If tissue specific viral activity is required, tissue specific promoters can be added to the viral genome to limit the viral expression to the specific type of cells and tissues.

### Example 3: In Vivo Humoral Immune Response to ONCoat-Ad5 Expressing SARS-CoV2 Spike Protein

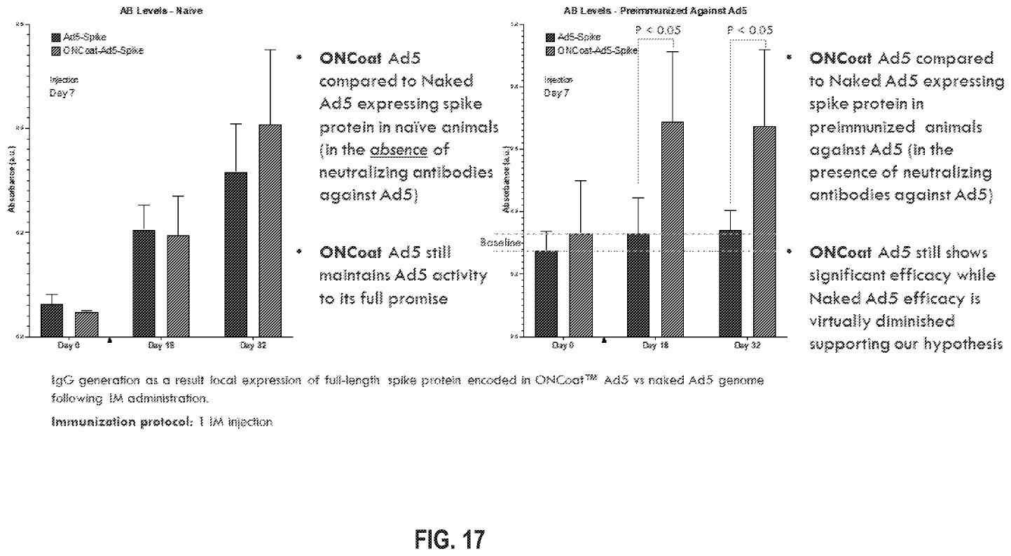

Mice that had or had not been previously exposed to Ad5 were injected once intramuscularly with uncoated Ad5 expressing SARS-CoV2 spike protein (i.e., naked Ad5-spike) or with ONCoat-treated Ad5 expressing SARS-CoV2 spike protein (i.e., surface-engineered virus or ONCoat-Ad5-spike). The humoral immune response was then evaluated at 7, 25, and 39 days after the injection by measuring levels of IgG antibodies against spike protein in the blood of the mice. FIG. 17 shows that in mice that had not been previously exposed to Ad5, the antibody levels were similar between animals injected with ONCoat-Ad5-spike and those injected with naked Ad5-spike. It would thus be expected that under normal conditions where a patient has not been previously exposed to Ad5, ONCoat-Ad5 would be at least as effective for vaccination as naked Ad5. FIG. 17 also shows that when mice were previously exposed to Ad5, thus resulting in the presence of neutralizing antibodies against Ad5, ONCoat-Ad5-spike still produced significant antibody levels. In contrast, the effect of naked Ad5 was almost entirely eliminated by the neutralizing antibodies resulting from previous exposure to Ad5. It would thus be expected that when a patient has been previously exposed to Ad5, whether through infection or through administration, ONCoat would maintain the vaccination capability of Ad5, even where the previous exposure would diminish the effect of naked Ad5.

### Example 4: In Vivo Cellular Immune Response to ONCoat-Ad5 Expressing SARS-CoV2 Spike Protein

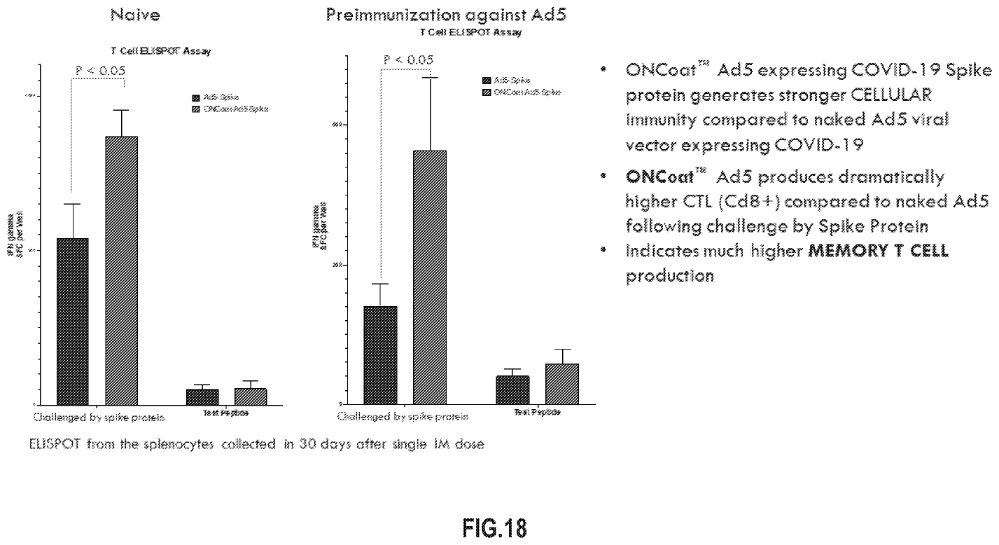

Mice that had or had not been previously exposed to Ad5 were injected once intramuscularly with uncoated Ad5 expressing SARS-CoV2 spike protein (i.e., naked Ad5-spike) or with ONCoat-treated Ad5 expressing SARS-CoV2 spike protein (i.e., surface-engineered virus or ONCoat-Ad5-spike). The cellular immune response was evaluated by T cell ELISPOT assay from splenocytes collected from the spleens of the mice when the mice were sacrificed 30 days after the injection. FIG. 18 shows higher interferon gamma production in the splenocyte ELISPOT for mice treated with ONCoat-Ad5-spike than for naked Ad5-spike upon challenge by spike protein, even for mice that had not been previously exposed to Ad5. This indicates an increase in CD8+ lymphocyte levels or activity or memory T cell levels or activity. It would thus be expected that even under normal conditions where a patient has not been previously exposed to Ad5, ONCoat-Ad5 would more effective for vaccination than naked Ad5. FIG. 18 also shows that when mice were previously exposed to Ad5, thus resulting in the presence of neutralizing antibodies against Ad5, the increase in interferon gamma production for ONCoat-Ad5-spike compared to naked Ad5 upon challenge by spike protein was even more pronounced, as the interferon gamma production for naked Ad5 was greatly reduced. It would thus be expected that when a patient has been previously exposed to Ad5, whether through infection or through administration, ONCoat would maintain the vaccination capability of Ad5, even where the previous exposure would diminish the effect of naked Ad5.

### Example 5: Application of ONCoat Technology on Ad26.COV2-S

### Experimental Overview

The objective of this proposed experiment study is to specifically evaluate the application of ONCoat technology with Ad26.COV2-S. This experiment demonstrates the applicability of ONCoat surface engineering technology, specifically with Ad26.COV2-S, and focuses on a number of benefits that ONCoat technology. The experiment is conducted in 3 parts. Application of ONCoat to Ad26.COV2-S is contemplated herein to: 1) prevent the neutralization of Ad26 vector thereby removing any impact of pre-existing immunity to the virus—expanding the population effectively immunized; 2) improve the vaccine efficiency by potentially reducing the vector dose required to elicit protective immunity; 3) reduce potential toxicity that may be related to vector dose and/or immune response to the vector; 4) enable effective boosting since a vector with ONCoat will not be impacted by any pre-existing or primary dose host immunogenicity to the vector particle; and 5) improve vector stability and reducing the cold chain requirements.

### Parts 1 & 2:

The physical characterization, biological activity in the presence and absence of neutralizing antibodies.
1. Initial QC and assay development for Ad26.COV2
2. Surface Engineering of Ad26.COV2 with ONCoat: ONCoat-Ad26-COV2
   a. Optimization of chemistry: Upon coating, the surface charge of the Ad26 is neutralized and the viral suspension is further stabilized as the coating provides steric stabilization. The ONCoat surface is functionalized with folic acid molecules to enable cell uptake and polyethylene glycol polymers to provide steric stability. Folic acid is an essential nutrient; and most human cells uptake folic acid efficiently.
   b. QC
      i. Viral particle quantification is conducted using qPCR
      ii. Optimization of protection assay
      iii. Viral activity evaluation—Transduction & Expression: in vitro—Protein Expression
      iv. Physical characterization: Size (DLS), surface charge (DLS), shape (SEM)
   c. Comparative Studies for Biological Activity: After optimization of the encapsulation procedure, biochemical characterization is performed and cell culture assays comparing material from the original virus stock with the encapsulated virus formulation to evaluate the improvement the surface engineered vector.

The cell culture experiments are used to compare the transduction and expression of SARS-CoV-2 S protein of the reengineered virus with control virus. For this set of experiments, the number of viral particles (determined by qPCR) is matched for both control group (Ad26.COV2) and reengineered group (ONCoat-Ad26-COV2). Experiments are performed both in the presence and absence of neutralizing antibodies against Ad26 vector using various antibody concentrations, of commercially available anti-Ad26 antibodies.

### Part 3:

1. Comparative In vivo administration
   a. Tox & PK & biodistribution are conducted
   b. Tissue antigen expression
   c. Immunological studies
   d. Evaluation of Booster shots/Repeated Administration
      i. Ad26.COV2-S, Ad26.COV2-S booster
      ii. Ad26.COV2-S, DevaCell booster
      iii. DevaCell, DevaCell booster

Ad26.COV2-S at a concentration of ˜1×10¹² vp/ml in cryoprotective buffer at −80° C. is provided in several aliquots (i.e. 0.1 ml) to avoid extra freeze/thaw cycles.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety to the same extent as if each individual publication, patent, or patent application was specifically and individually indicated to be incorporated by reference.

What is claimed is:

1. A replication-competent oncolytic virus comprising:
a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme.

2. The replication-competent oncolytic virus of claim 1, wherein each transgene expresses a different protein.

3. The replication-competent oncolytic virus of claim 1-2, wherein the first transgene expresses GM-CSF, IL-12, IL-15, TNF-α, or 4-1BBL.

4. The replication-competent oncolytic virus of claim 1-3, wherein the second transgene expresses CCL4, CCL5/RANTES, CXCL11, 4-1BBL, GM-CSF, or TNF-α.

5. The replication-competent oncolytic virus of claim 1-4, wherein the third transgene expresses an essential-agent-depletion-enzyme selected from methioninase, adenosine deaminase, or cytosine deaminase, asparaginase or uricase.

6. The replication-competent oncolytic virus of claim 1-5, wherein the first transgene expresses GM-CSF, IL-12, IL-15, TNF-α, or 4-1BBL; the second transgene expresses CCL4, CCL5/RANTES, CXCL11, 4-1BBL, GM-CSF, or TNF-α; and the third transgene expresses methioninase, adenosine deaminase, or cytosine deaminase.

7. The replication-competent oncolytic virus of claim 1-6, wherein the third transgene is expressed before the other two transgenes.

8. The replication-competent oncolytic virus of claim 1-7, wherein the third transgene is expressed under control of an E1 or E3 promoter.

9. The replication-competent oncolytic virus of claim 1-7, wherein the virus is selected from the group consisting of: adeno-associated virus (AAV), adenovirus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus, Enadenotucirev oncolytic virus, Ad26, Imlygic, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus, Pexa-Vec, Reolysin, DS-1647, TG1042, Cavatak, GL-ONC1, Marabex, ORCA-010, ParvOryx, LOAd703, PV701, MV-NIS, ONCOS-102, Seprehvir, Enadenotucirev, CG0070, Telomelysin, JX-929, VSV Cancer Project, Ad-VirRx 007, NG-348, VSV-GP, RP1/RP2/RP3, WO-12, Maraba virus, Caraj as virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, Bovine ephemeral fever virus and/or Dimarhabdovirus.

10. The replication-competent oncolytic virus of claim 1-9, wherein the virus is an adenovirus.

11. The replication-competent oncolytic virus of claim 10, wherein the adenovirus further comprises a 24-bp deletion in E1A Conserved Region 2 (CR2), and a 2428 bp BsiWI-BglII deletion in the E3 region.

12. The replication-competent oncolytic virus of claim 1-11, wherein the first, second, and third transgenes are selected from the group of first, second and third transgene combinations set forth in Table 1 and FIGS. 19-21.

13. The replication-competent oncolytic virus of claim 1-12, wherein the first, second, and third transgenes are selected from the group of first, second and third transgene combinations selected from:

| | | | |
|---|---|---|---|
| Ad5.DEV09 | RFP | hCCL5/RANTES-human | IL15-human |
| Ad5.DEV10 | Adenosine Deaminase-human | hCCL5/RANTES-human | IL15-human |
| Ad5.DEV11 | Methioninase | hCCL5/RANTES-human | IL15-human |
| Ad5.DEV12 | Methioninase | Adenosine Deaminase-human | IL15-human |
| Ad5.DEV13 | Methioninase | Adenosine Deaminase-human | GM-CSF-mouse |
| Ad5.DEV14 | Adenosine Deaminase-human | GM-CSF-mouse | IL15-human |
| Ad5.DEV15 | Methioninase | GM-CSF-mouse | IL15-human |

14. A replication-competent oncolytic virus comprising:
a first, and a second transgene, whereas each transgene expresses GM-CSF, IL-12, IL-15, TNF-α, 4-1BBL, CCL4, CCL5/RANTES, CXCL11, or an essential-agent-depletion-enzyme, wherein each transgene expresses a different protein, and wherein the first and second transgenes are selected from the group of first and second transgene combinations set forth in Table 1 and FIGS. 19-21.

15. A method of treating cancer in an individual in need thereof, comprising administering to the individual a replication-competent oncolytic virus of claims 1-14, or a surface-engineered recombinant oncolytic virus; thereby treating the individual.

16. The method of claim 15, wherein the surface-engineered recombinant oncolytic virus comprises the replication-competent oncolytic virus of claims 1-14.

17. The method of claims 15-16, wherein the replication-competent oncolytic virus or surface-engineered recombinant oncolytic virus is administered in combination with or as an adjuvant to another anti-cancer drug.

18. The method of claims 15-17, wherein the replication-competent oncolytic virus or surface-engineered recombinant oncolytic virus is administered once as a single dose, or repeatedly at 2 or more intervals.

19. A method of administering a gene therapy to an individual in need thereof, comprising administering to the individual a surface-engineered recombinant gene-therapy virus comprising a transgene encoding a therapeutic protein; thereby administering the gene therapy to the individual.

20. The method of claim 19, wherein the surface-engineered recombinant gene-therapy virus is administered by intramuscular, intravenous, intracranial, or intrathecal injection, or injection into any tissue where transgene expression is desired.

21. The method of claims 19-20, wherein the surface-engineered recombinant gene-therapy virus is administered once as a single dose, or repeatedly at 2 or more intervals.

22. A surface-engineered recombinant virus, said virus comprising:
a recombinant virus having a recombinant genome, or a replication-competent oncolytic virus of claims 1-14; and
an artificial coating layer surrounding the recombinant virus.

23. The surface-engineered recombinant virus of claim 22, wherein the artificial coating is selected from the group consisting of: silica, titanium oxide and calcium phosphate.

24. The surface-engineered recombinant virus of claims 22-23, wherein the virus is selected from the group consisting of: enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, and hepatitis A virus.

25. The surface-engineered recombinant virus of claims 22-24, wherein the recombinant virus is oncolytic and is selected from the group consisting of: NG-641 (PsiOxus), and Imlygic (talimogene laherparepvec).

26. The surface-engineered recombinant virus of claims 22-24, wherein the virus is a vaccine and is selected from AZD1222 (AstraZeneca), ChAdOx1-nCov19 (Oxford), Ad5-nCoV (CanSino), VSV, and Ad26 (J&J).

27. The surface-engineered recombinant virus of claim 22, wherein the virus is replication deficient Ad5 (Human) Adenovirus vector, and wherein the recombinant genome encodes SARS-CoV-2 spike protein and E1 & E3 genes are deleted; or wherein the adenovirus further comprises a 24-bp deletion in E1A Conserved Region 2 (CR2), and a 2428 bp BsiWI-BglII deletion in the E3 region; or wherein the virus does not contain any native Ad5 viral genes.

28. The surface-engineered recombinant virus of claim 22, wherein the virus is oncolytic and the virus is VSV.

29. The surface-engineered recombinant virus of claims 22-28, wherein the artificial coating is applied by conducting a charge-mediated sol-gel condensation reaction directly onto the surface of the recombinant virus.

30. The surface-engineered virus of claims 22-29, wherein the artificial coating comprises a silica gel matrix or titanium oxide gel matrix encapsulating the recombinant virus.

31. The surface-engineered virus of claims 22-30, wherein the artificial coating comprises a targeting-ligand selected from the group consisting of: proteins, polysaccharides, aptamers, peptides, oligonucleotides and small molecules.

32. The surface-engineered virus of claims 22-31, wherein the artificial coating comprises a targeting-ligand selected from the group consisting of: Antibodies, transferrin, Hyaluronic acid, RGD, IL4RPep-1, AS-1411, GBI-10, Folate, anisamide, and phenylboronic acid.

33. The surface-engineered virus of claims 22-32, wherein the recombinant virus is replication-competent or replication-defective.

34. The surface-engineered virus of claims 22-33, wherein the recombinant virus has had it native-envelope removed prior to coating with the artificial coating layer.

35. A surface-engineered recombinant virus vaccine, said virus comprising:
a recombinant Ad5 virus having a recombinant genome encoding SARS-CoV-2 spike protein, wherein at least a functional portion of E1 and E3 genes are deleted, or wherein the adenovirus further comprises a 24-bp deletion in E1A Conserved Region 2 (CR2), and a 2428 bp BsiWI-BglII deletion in the E3 region; or wherein the virus does not contain any native Ad5 viral genes;
a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating layer encapsulating the recombinant Ad5 virus, wherein said coating layer comprises an effective amount of folate to bind a folate receptor on a cell, and wherein the artificial coating is selected from the group consisting of: silica, titanium oxide and calcium phosphate.

36. A method of making a surface-engineered recombinant virus, said method comprising:
producing a recombinant virus having a recombinant genome, or a replication-competent oncolytic virus of claims 1-14; and
applying an artificial coating to the recombinant virus, wherein the artificial coating is selected from the group consisting of: silica, titanium oxide and calcium phosphate.

37. The method of claim 36, wherein the artificial coating is applied by conducting a charge-mediated sol-gel condensation reaction directly onto the surface of the recombinant virus.

38. The method of claims 36-37, wherein the artificial coating comprises a silica gel matrix or titanium oxide gel matrix encapsulating the recombinant virus.

39. The method of claims 36-38, wherein the recombinant virus has had its native-envelope removed prior to coating with the artificial coating layer.

40. A method of re-engineering the surface of a virus having a native-envelope, said method comprising:
removing the native-envelope from the virus to isolate a previously-enveloped-capsid;
applying an artificial coating to the previously-enveloped-capsid, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme.

41. The method of claim 40, wherein the native-envelope virus is selected from the group consisting of: Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

42. The method of claims 40-41, wherein the native-envelope is removed or delipidated using a detergent and/or an extraction solvent.

43. The method of claim 42, wherein the detergent and/or an extraction solvent is selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination.

44. The method of claims 40-43, wherein the artificial coating is selected from the group consisting of: silica, titanium oxide and calcium phosphate.

45. The method of claims 40-44, wherein applying the artificial coating further comprises conducting a charge-mediated sol-gel condensation reaction directly onto the surface of the previously-enveloped-capsid.

46. The method of claims 40-45, wherein the artificial coating comprises a silica gel matrix or titanium oxide gel matrix encapsulating the previously-enveloped-capsid.

47. The method of claims 45-46, wherein the previously-enveloped-capsid is replication-competent or replication-defective.

48. A surface-re-engineered virus comprising:
a previously-enveloped-capsid from a naturally occurring enveloped-virus, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating layer surrounding the previously-enveloped-capsid.

49. The surface-re-engineered virus of claim 48, wherein the envelope virus is selected from the group consisting of: Poxvirus, vaccinia virus, Herpes Virus, herpes simplex virus-1, herpes simplex virus-2, Rhabdovirus, Vesicular stomatitis virus (VSV), Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

50. The surface-re-engineered virus of claims 48-49, wherein the artificial coating is selected from the group consisting of: silica, titanium oxide and calcium phosphate.

51. The surface-re-engineered virus of claims 48-50, wherein the artificial coating is applied by conducting a charge-mediated sol-gel condensation reaction directly onto the surface of the previously-enveloped-capsid,

52. The surface-re-engineered virus of claims 48-51, wherein the artificial coating comprises a silica gel matrix or titanium oxide gel matrix encapsulating the previously-enveloped-capsid.

53. The surface-re-engineered virus of claims 48-52, wherein the previously-enveloped-capsid is replication-competent or replication-defective.

54. A method of re-engineering a virus having a native-envelope, said method comprising:
removing the native-envelope surrounding a capsid from the virus, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme;
isolating the previously-enveloped-capsid; and
applying an artificial coating to the previously-enveloped-capsid.

55. A composition comprising;
a capsid from a native envelope-virus, wherein the capsid is devoid of its native envelope, wherein said virus comprises a first, a second, and a third transgene, whereas each transgene expresses GM-CSF, IL-12, IL-15, TNF-α, 4-1BBL, CCL4, CCL5/RANTES, CXCL11, or an essential-agent-depletion-enzyme; and
an artificial coating-layer, wherein the coating-layer encapsulates the capsid.

56. The composition of claim 55, wherein the envelope-virus is selected from the group consisting of: Herpesviruses, Poxviruses (e.g., vaccinia virus), Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus, and Retroviruses.

57. The composition of claims 55-56, wherein the coating-layer protects the capsid from immune recognition and neutralization during therapy.

58. The composition of claims 55-57, wherein the coating-layer further comprises binding agents on its surface that changes the infectivity and/or biological activity of the native envelope-virus.

59. A method of making the composition of claims 55-58, comprising removing the envelope of an envelope virus to produce an envelope-free-capsid; and encapsulating the envelope-free-capsid with an artificial coating-layer.

60. A surface-engineered delivery system, said system comprising:
a payload, or a replication-competent oncolytic virus of claims 1-14; and
an artificial coating layer surrounding the payload.

61. The surface-engineered delivery system of clam 60, wherein the payload is selected from a recombinant virus or a nucleic acid.

62. The surface-engineered delivery system of claims 60-61, wherein the artificial coating is selected from the group consisting of: silica, titanium oxide and calcium phosphate.

63. The surface-engineered recombinant virus of claim 27, wherein the coating of the virus increases humoral immunity induced by the virus against SARS-CoV-2 spike protein.

64. The surface-engineered recombinant virus of claim 63, wherein the increased humoral immunity is characterized by increased IgG antibody levels.

65. The surface-engineered recombinant virus of claim 27, wherein the coating of the virus increases cellular immunity induced by the virus against SARS-CoV-2 spike protein.

66. The surface-engineered recombinant virus of claim 65, wherein the increased cellular immune is characterized by increased CD8+ lymphocyte levels.

67. The surface-engineered recombinant virus of claim 65, wherein the increased cellular immunity is characterized by increased memory T cell levels.

68. The surface-engineered recombinant virus vaccine of claim 35, wherein the coating of the virus increases humoral immunity induced by the virus against SARS-CoV-2 spike protein.

69. The surface-engineered recombinant virus vaccines of claim 68, wherein the increased humoral immunity is characterized by increased IgG antibody levels.

70. The surface-engineered recombinant virus vaccine of claim 35, wherein the coating of the virus increases cellular immunity induced by the virus against SARS-CoV-2 spike protein.

71. The surface-engineered recombinant virus of claim 70, wherein the increased cellular immune is characterized by increased CD8+ lymphocyte levels or activity.

72. The surface-engineered recombinant virus of claim 70, wherein the increased cellular immunity is characterized by increased memory T cell levels or activity.

73. The surface-engineered recombinant virus of claim 27, wherein the coating of the virus increases immunity induced by the virus against SARS-CoV-2 spike protein in an individual having pre-existing neutralizing antibodies against Ad5 adenovirus.

74. The surface-engineered recombinant virus of claim 73, wherein the increased immunity is characterized by increased IgG antibody levels.

75. The surface-engineered recombinant virus of claim 73, wherein the increased immunity is characterized by increased CD8+ lymphocyte levels or activity.

76. The surface-engineered recombinant virus of claim 73, wherein the increased immunity is characterized by increased memory levels or activity.


SEQUENCE LISTING (18 sequences)

SEQ ID NO: 1 - DNA, 795 bp, Unknown
  Description of Unknown Synthetic Construct
  CDS (1)..(795)
atggtgtcgaagggagaagaactgattaaggaaaacatgcggatgaaagtggtgatggaagggtcggtgaatggacaccaattcaagtgcaccggagagggagagggcaacccatacatgggtactcagaccatgcgcatcaaggtcgttgaaggaggacctctgccttttgcgttcgacatccttgctacctcgttcatgtacgggtcccgcacctttatcaagtacccgaagggaatcccggatttcttcaagcagagcttcccggaaggattcacctgggagagggtgactcggtacgaagatggaggagtgctgactgcaacccaagacacttcgctcgaggacggctgtctggtgtaccatgtccaagtgcggggtgtgaacttcccctcaaatgggccagtgatgcagaaaaagaccctcggatgggaagcgaacaccgagatgatgtacccggcggacggtggcttgcgaggatacactcacatggccttgaagctggacggcggaggtcatctctcatgctcctttgtcactacctaccgcagcaagaaaactgtcggaaacatcaagatgccgggcgtgtactacgtcgatcaccggctcgagagaatcaaagaggccgacaaggaaacgtatgtcgagcagcatgaagtcgcagtggccaggtactgcgaccttccctccaaactgggccacaagctgaattctggcctgagaagccgcgcacaggcttcgaactcagccgtggatgggacggccggcccagggtccactggaagcagaggttga

SEQ ID NO: 2 - protein, 264 aa, Unknown
  Synthetic Construct
MVSKGEELIKENMRMKVVMEGSVNGHQFKCTGEGEGNPYMGTQTMRIKVVEGGPLPFAFDILATSFMYGSRTFIKYPKGIPDFFKQSFPEGFTWERVTRYEDGGVLTATQDTSLEDGCLVYHVQVRGVNFPSNGPVMQKKTLGWEANTEMMYPADGGLRGYTHMALKLDGGGHLSCSFVTTYRSKKTVGNIKMPGVYYVDHRLERIKEADKETYVEQHEVAVARYCDLPSKLGHKLNSGLRSRAQASNSAVDGTAGPGSTGSRG

SEQ ID NO: 3 - DNA, 1092 bp, Unknown
  Description of Unknown Synthetic Construct
  CDS (1)..(1092)
atggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 4 - protein, 363 aa, Unknown
  Synthetic Construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 5 - DNA, 1197 bp, Mus musculus
  CDS (1)..(1197)
atgcacggctccaacaagctcccaggatttgccacccgcgccattcaccatggctacgacccccaggaccacggcggcgcactggtgccaccggtctaccagaccgcgacgttcaccttccccaccgtggaatacggcgctgcgtgctttgccggcgagcaggccggccatttctacagccgcatctccaaccccaccctcaacctgctggaagcacgcatggcctcgctggaaggcggcgaggccgggctggcgctggcctcgggcatgggggcgatcacgtccacgctatggacactgctgcgccccggtgacgaggtgctgctgggcaacaccctgtacggctgcacctttgccttcctgcaccacggcatcggcgagttcggggtcaagctgcgccatgtggacatggccgacctgcaggcactggaggcggccatgacgccggccacccgggtgatctatttcgagtcgccggccaaccccaacatgcacatggccgatatcgccggcgtggcgaagattgcacgcaagcacggcgcgaccgtggtggtcgacaacacctactgcacgccgtacctgcaacggccactggagctgggcgccgacctggtggtgcattcggccaccaagtacctgagcggccatggcgacatcactgctggcattgtggtgggcagccaggcactggtggaccgtatacgtctgcagggcctcaaggacatgaccggtgcggtgctctcgccccatgacgccgcactgttgatgcgcggcatcaagaccctcaacctgcgcatggaccgccactgcgccaacgctcaggtgctggccgagttcctcgcccggcagccgcaggtggagctgatccattacccgggcctggcgagcttcccgcagtacaccctggcccgccagcagatgagccagccgggcggcatgatcgccttcgaactcaagggcggcatcggtgccgggcggcggttcatgaacgccctgcaactgttcagccgcgcggtgagcctgggcgatgccgagtcgctggcgcagcacccggcaagcatgactcattccagctataccccagaggagcgtgcgcattacggcatctccgaggggctggtgcggttgtcggtggggctggaagacatcgacgacctgctggccgatgtgcaacaggcactcaaggcgagtgcctga

SEQ ID NO: 6 - protein, 398 aa, Mus musculus
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 7 - DNA, 1197 bp, Mus musculus
  CDS (1)..(1197)
atgcatggatcaaataagcttcccggcttcgcaacacgggcaatccatcatggctatgatcctcaagatcatggaggtgccctggttccccctgtttaccagactgctacctttacattccctaccgtagagtatggtgccgcttgctttgctggagaacaagccggtcacttctattcacgaattagtaatccaaccctgaatctgcttgaggcccgcatggcatcactggaaggaggggaagctggcctggcattggcatctggtatgggagctatcacatccacattgtggaccttgttgcggcccggagacgaagttctcctgggaaatacactgtatggttgcacatttgcattcctccatcatggcattggtgaattcggagtgaagcttcggcatgtagacatggcagacctccaagccctcgaagctgccatgaccccagcaaccagagtcatatactttgaatccccagccaatccaaacatgcatatggcagacatagctggcgttgctaagatcgccagaaagcatggagccactgtcgttgtggacaatacatattgcacaccataccttcaaaggcctttggaattgggcgccgacctggttgttcattcagcaactaaatatctctctggccatggagacataacagccggaatagtagtaggatctcaagcacttgtagataggatcagactgcaaggccttaaagatatgactggggccgtactcagtccacacgatgcagcactgttgatgaggggaatcaagacccttaaccttagaatggacaggcactgtgcaaacgctcaggtactcgctgagtttctggcacgccaaccccaggtggaactcattcactatcccggtttggctagtttccctcaatacaccctggctcgacagcagatgtctcagcctgggggcatgatcgctttcgagttgaaaggcggcatcggggccggtcggcggtttatgaacgcactccaactcttttcaagggcagtcagtcttggagacgctgaaagtttggcacagcacccagcatctatgactcactcctcttatacaccagaggaacgagcccactatggaatctctgaaggcctggtaaggcttagtgtcggactggaagatattgatgatctgcttgcagacgttcagcaagcacttaaagcctctgcttga

SEQ ID NO: 8 - protein, 398 aa, Mus musculus; identical to SEQ ID NO: 6

SEQ ID NO: 9 - DNA, 648 bp, Unknown
  Description of Unknown Synthetic Construct
  CDS (1)..(648)
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 10 - protein, 215 aa, Unknown
  Synthetic Construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 11 - DNA, 1008 bp, Unknown
  Description of Unknown Synthetic Construct
  CDS (1)..(1008)
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 12 - protein, 335 aa, Unknown
  Synthetic Construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 13 - DNA, 489 bp, Unknown
  Description of Unknown Synthetic Construct
  CDS (1)..(489)
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 14 - protein, 162 aa, Unknown
  Synthetic Construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 15 - DNA, 426 bp, Unknown
  Description of Unknown Synthetic Construct
  CDS (1)..(426)
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatga

SEQ ID NO: 16 - protein, 141 aa, Unknown
  Synthetic Construct
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 17 - DNA, 276 bp, Unknown
  Description of Unknown Synthetic Construct
  CDS (1)..(276)
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 18 - protein, 91 aa, Unknown
  Synthetic Construct
MKVSAAALAVILIATALCAPASASPYSSDTTPCCFAYIARPLPRAHIKEYFYTSGKCSNPAVVFVTRKNRQVCANPEKKWVREYINSLEMS